SEQUENCE LISTING (79 sequences)

SEQ ID NO: 1 - DNA, 1104 bp, Artificial Sequence
  synthetic construct
  exon (1)..(89)
  sig_peptide (1)..(63)
  CDS (1)..(89)
  mat_peptide (64)..(1101)
  Intron (90)..(167)
  exon (168)..(416)
  CDS (168)..(416)
  Intron (417)..(485)
  exon (486)..(1101)
  CDS (486)..(1101)
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaagcgcaacgacgatggtaacttccctgtgttcacccctccttcgggaggccagtcctcctcgtcctcctcgtcctcgtcggccaagccgacatcgacgtcgacgtccactacttcgaccaaagcaacctccacaacttccaccgcctcgtcccagacgtcgtcgtccaccggaggaggctgcgcagcgcagcgttgggctcagtgcggtggcatcggcttcagcggatgcaccacctgtgtctctggcaccacctgcaacaagcagaacgactggtactcgcagtgcctctga

SEQ ID NO: 2 - protein, 318 aa, Artificial Sequence
  Synthetic Construct
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDGNFPVFTPPSGGQSSSSSSSSSAKPTSTSTSTTSTKATSTTSTASSQTSSSTGGGCAAQRWAQCGGIGFSGCTTCVSGTTCNKQNDWYSQCL

SEQ ID NO: 3 - DNA, 1297 bp, Artificial Sequence
  Synthetic construct
  sig_peptide (1)..(63)
  exon (1)..(89)
  CDS (1)..(89)
  mat_peptide (64)..(1101)
  Intron (90)..(167)
  exon (168)..(416)
  CDS (168)..(416)
  Intron (417)..(485)
  exon (486)..(1101)
  CDS (486)..(1101)
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaagcgcaacgacgatggtaacttccctgtgttcacccctccttcgggaggccagtcctcctcgtcctcctcgtcctcgtcggccaagccgacatcgacgtcgacgtccactacttcgaccaaagcaacctccacaacttccaccgcctcgtcccagacgtcgtcgtccaccggaggaggctgcacgtctcagaagtgggctcagtgcggtggcatcggcttcagcggatgcaccacctgtgtctctggcaccacctgccagaagttgaacgactactactcgcagtgcctctaaacagcttttcgcacgaggtggcgggacggagcaaggagaccgtcaacttcgtcatgcatattttttgagcgctcaatacatacataaccttcgattcttgtacatagcacgccggtacacatctcacaccgactttgggggcggaatcaggcccgttttaccccgagttcgctgcgactgctggttgatttcc

SEQ ID NO: 4 - protein, 318 aa, Artificial Sequence
  Synthetic Construct
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDGNFPVFTPPSGGQSSSSSSSSSAKPTSTSTSTTSTKATSTTSTASSQTSSSTGGGCTSQKWAQCGGIGFSGCTTCVSGTTCQKLNDYYSQCL

SEQ ID NO: 5 - DNA, 1104 bp, Artificial Sequence
  Synthetic construct
  sig_peptide (1)..(63)
  exon (1)..(89)
  CDS (1)..(89)
  mat_peptide (64)..(1101)
  Intron (90)..(167)
  exon (168)..(416)
  CDS (168)..(416)
  Intron (417)..(485)
  exon (486)..(1101)
  CDS (486)..(1101)
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaagcgcaacgacgatggtaacttccctgtgttcacccctccttcgggaggccagtcctcctcgtcctcctcgtcctcgtcggccaagccgacatcgacgtcgacgtccactacttcgaccaaagcaacctccacaacttccaccgcctcgtcccagacgtcgtcgtccaccggaggaggctgcgcagcgcagcgttgggctcagtgcggtggcatcggcttcagcggatgcaccacctgtgtctctggcaccacctgcaacaagcagaacgactactactcgcagtgcctctga

SEQ ID NO: 6 - protein, 318 aa, Artificial Sequence
  Synthetic Construct
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDGNFPVFTPPSGGQSSSSSSSSSAKPTSTSTSTTSTKATSTTSTASSQTSSSTGGGCAAQRWAQCGGIGFSGCTTCVSGTTCNKQNDYYSQCL

SEQ ID NO: 7 - DNA, 1071 bp, Artificial Sequence
  Synthetic construct
  sig_peptide (1)..(63)
  exon (1)..(89)
  CDS (1)..(89)
  mat_peptide (64)..(1068)
  Intron (90)..(167)
  exon (168)..(416)
  CDS (168)..(416)
  Intron (417)..(485)
  exon (486)..(1068)
  CDS (486)..(1068)
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaaggcgaacgacgactccagcttccccgtcttcaccccgggcacctcgggctcgtcctcgaccgccaagcccgcttcctcttcgacccgggccacgtccacgaagacctcggctcctgcgacccagacgtcgtcgactggaggcggctgcgtcgcgcagaagtgggcgcaatgtggtggtagcggattttctggctgcaccacctgcgctgccgggtcgacgtgcacgaagcagaacgactactactcgcagtgcctctaa

SEQ ID NO: 8 - protein, 307 aa, Artificial Sequence
  Synthetic Construct
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKANDDSSFPVFTPGTSGSSSTAKPASSSTRATSTKTSAPATQTSSTGGGCVAQKWAQCGGSGFSGCTTCAAGSTCTKQNDYYSQCL

SEQ ID NO: 9 - DNA, 1071 bp, Artificial Sequence
  synthetic construct
  sig_peptide (1)..(63)
  exon (1)..(89)
  CDS (1)..(89)
  mat_peptide (64)..(1068)
  Intron (90)..(167)
  exon (168)..(416)
  CDS (168)..(416)
  Intron (417)..(485)
  exon (486)..(1068)
  CDS (486)..(1068)
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaaggcgaacgacgactccagcttccccgtcttcaccccgggcacctcgggctcgtcctcgaccgccaagcccgcttcctcttcgacccgggccacgtccacgaagacctcggctcctgcgacccagacgtcgtcgactggaggcggctgcgtcgcgcagaagtgggcgcaatgtggtggtagcggattttctggctgcaccacctgcgctgccgggtcgacgtgcacgaagcagaacgactggtactcgcagtgcctctaa

SEQ ID NO: 10 - protein, 307 aa, Artificial Sequence
  Synthetic Construct
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKANDDSSFPVFTPGTSGSSSTAKPASSSTRATSTKTSAPATQTSSTGGGCVAQKWAQCGGSGFSGCTTCAAGSTCTKQNDWYSQCL

SEQ ID NO: 11 - DNA, 1104 bp, Artificial Sequence
  synthetic construct
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaagcgcaacgacgatggtaacttccctgtgttcacccctccttcgggaggccagtcctcctcgtcctcctcgtcctcgtcggccaagccgacatcgacgtcgacgtccactacttcgaccaaagcaacctccacaacttccaccgcctcgtcccagacgtcgtcgtccaccggaggaggctgtgcagcgcagcgttgggcacagtgtggtggtatcggattctcgggatgtactacatgtgtgtcgggcacgacgtgtaacaagcagaacgactggtactcccagtgtctctga

SEQ ID NO: 12 - protein, 299 aa, Thielavia terrestris
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDSSFPVFTPPSGGNGGTGTPTSTAPGSGQTSPGGGSGCTSQKWAQCGGIGFSGCTTCVSGTTCQKLNDYYSQCL

SEQ ID NO: 13 - protein, 295 aa, Staphylotrichum coccosporum
ADGKSTRYWDCCKPSCSWPGKASVNQPVFACSANFQRISDPNVKSGCDGGSAYACADQTPWAVNDNFSYGFAATSISGGNEASWCCGCYELTFTSGPVAGKTMVVQSTSTGGDLGTNHFDLAMPGGGVGIFDGCSPQFGGLAGDRYGGVSSRSQCDSFPAALKPGCYWRFDWFKNADNPTFTFRQVQCPSELVARTGCRRNDDGNFPVFTPPSGGQSSSSSSSSSAKPTSTSTSTTSTKATSTTSTASSQTSSSTGGGCAAQRWAQCGGIGFSGCTTCVSGTTCNKQNDWYSQCL

SEQ ID NO: 14 - protein, 293 aa, Neurospora tetrasperma
MRSSTVLQTGLVAALPFAVQAASGSGQSTRYWDCCKPSCSWSGKASVNRPVLACDANNNPLSDASVKSGCDGGSAYTCANNSPWAVNDQLSYGFAATKLSGGTESSWCCACYALTFTSGPVAGKTMVVQSTSTGGDLGSNHFDINMPGGGVGLFDGCTRQFGGLPGAQYGGISSRSQCDSFPAALKPGCQWRFDWFQNADNPNFTFKQVQCPSELTSRTGCKRNDDSQFPVFTPPSGGGTNPSTPTTPPSSGGGSGCTADKYAQCGGSGWSGCTNCPSGSTCKTINDYYHQCA

SEQ ID NO: 15 - DNA, 951 bp, Artificial Sequence
  synthetic construct
  sig_peptide (1)..(63)
  exon (1)..(948)
  CDS (1)..(948)
  mat_peptide (64)..(948)
atgcgctcctcccctgtcctcaggacagcgctcgcagcagccctccccctcgcagcgttggcagcagatggaaagtcgacacgctattgggactgttgtaagccttcgtgttcgtggcctggcaaggcatccgtgaaccagccggtcttcgcatgttccgcaaacttccagcggatctccgatccgaacgtcaaatccggttgtgatggaggctccgcctacgcgtgtgccgaccagacaccttgggcagtcaacgacaacttctcctacggcttcgcagcaacttcgatctcgggtggcaacgaggcctcgtggtgttgtggatgttacgaactcaccttcacttcgggacctgtggcaggtaagaccatggtcgtccagtcgacgtcgacaggaggcgacctcggtaccaaccacttcgatttggcgatgcctggaggaggcgtgggcatcttcgacggctgttcgcctcagttcggaggcttggcaggcgaccggtatggcggtgtgtcctcccggtcgcagtgtgattccttccctgcagccctcaaacccggttgttactggcgattcgactggttcaagaacgccgacaaccctaccttcacgttccgacaggtgcagtgtccgtcggagttggtcgccaggaccggctgtcgcaggaacgatgatggtaacttccctgtgttcacccctccttcgggaggccagtcctcctcgtcctcctcgtcctcgtcggccaagccgacatcgacgtcgacgtccactacttcgaccaaagcaacctccacaacttccaccgcctcgtcccagacgtcgtcgtccaccggaggaggctgtgcagcgcagcgttgggcacagtgtggtggtatcggattctcgggatgtactacatgtgtgtcgggcacgacgtgtaacaagcagaacgactactactcccagtgtctctaa

SEQ ID NO: 16 - protein, 316 aa, Artificial Sequence
  Synthetic Construct
MRSSPVLRTALAAALPLAALAADGKSTRYWDCCKPSCSWPGKASVNQPVFACSANFQRISDPNVKSGCDGGSAYACADQTPWAVNDNFSYGFAATSISGGNEASWCCGCYELTFTSGPVAGKTMVVQSTSTGGDLGTNHFDLAMPGGGVGIFDGCSPQFGGLAGDRYGGVSSRSQCDSFPAALKPGCYWRFDWFKNADNPTFTFRQVQCPSELVARTGCRRNDDGNFPVFTPPSGGQSSSSSSSSSAKPTSTSTSTTSTKATSTTSTASSQTSSSTGGGCAAQRWAQCGGIGFSGCTTCVSGTTCNKQNDYYSQCL

SEQ ID NO: 17 - DNA, 894 bp, Artificial Sequence
  synthetic construct
  sig_peptide (1)..(63)
  exon (1)..(891)
  CDS (1)..(891)
  mat_peptide (64)..(891)
atgcgctcctcccctgtcctcaggacagcgctcgcagcagccctccccctcgcagcgttggcagcagatggaaagtcgacacgctattgggactgttgtaagccttcgtgttcgtggcctggcaaggcatccgtgaaccagccggtcttcgcatgttccgcaaacttccagcggatctccgatccgaacgtcaaatccggttgtgatggaggctccgcctacgcgtgtgccgaccagacaccttgggcagtcaacgacaacttctcctacggcttcgcagcaacttcgatctcgggtggcaacgaggcctcgtggtgttgtggatgttacgaactcaccttcacttcgggacctgtggcaggtaagaccatggtcgtccagtcgacgtcgacaggaggcgacctcggtaccaaccacttcgatttggcgatgcctggaggaggcgtgggcatcttcgacggctgttcgcctcagttcggaggcttggcaggcgaccggtatggcggtgtgtcctcccggtcgcagtgtgattccttccctgcagccctcaaacccggttgttactggcgattcgactggttcaagaacgccgacaaccctaccttcacgttccgacaggtgcagtgtccgtcggagttggtcgccaggaccggctgtcgcgcaaacgacgactccagcttccccgtcttcacccccccaagcggtggcaacggtggcaccgggacgcccacgtcgactgcgcctgggtcgggccagacgtctcccggcggcggcagtggctgcacgtctcagaagtgggctcagtgcggtggcatcggcttcagcggatgcaccacctgtgtctctggcaccacctgccagaagttgaacgactactactcgcagtgcctctaa

SEQ ID NO: 18 - protein, 297 aa, Artificial Sequence
  Synthetic Construct
MRSSPVLRTALAAALPLAALAADGKSTRYWDCCKPSCSWPGKASVNQPVFACSANFQRISDPNVKSGCDGGSAYACADQTPWAVNDNFSYGFAATSISGGNEASWCCGCYELTFTSGPVAGKTMVVQSTSTGGDLGTNHFDLAMPGGGVGIFDGCSPQFGGLAGDRYGGVSSRSQCDSFPAALKPGCYWRFDWFKNADNPTFTFRQVQCPSELVARTGCRANDDSSFPVFTPPSGGNGGTGTPTSTAPGSGQTSPGGGSGCTSQKWAQCGGIGFSGCTTCVSGTTCQKLNDYYSQCL

SEQ ID NO: 19 - DNA, 935 bp, Artificial Sequence
  synthetic construct
  sig_peptide (1)..(63)
  exon (1)..(338)
  CDS (1)..(338)
  mat_peptide (64)..(932)
  Intron (339)..(391)
  exon (392)..(932)
  CDS (392)..(932)
atgcgctcctccactgttctgcaaaccgggctagtggccgctcttcccttcgccgttcaggccgcgtccggatccggccagtccaccagatattgggactgctgcaaaccatcttgctcctggtccggcaaggcttctgtcaaccgacccgtcctcgcttgcgatgcaaacaacaaccccctgagcgacgccagtgtcaagtctggatgtgacggcggttctgcatacacctgtgccaacaactcaccatgggcggtgaacgaccagctctcctacggctttgctgccacgaaactcagtggtggaaccgagtcgtcttggtgctgtgcctgttatgcgtgagtttcgcgtcaacagctgggtatcacttgatggctaatttatactgcagccttaccttcacttcgggtcctgttgctggcaagaccatggtcgttcagtctaccagtaccggcggtgatctcggctccaaccacttcgatatcaacatgcccggcggcggcgtcggcctgtttgatggctgtacacgacagtttggcggtctccccggcgctcaatatggcggcatcagctcccgcagccagtgcgattcattccctgccgcgctcaagcccggttgccagtggcgcttcgactggttccagaacgccgacaaccccaacttcaccttcaagcaggtccaatgcccatccgagctcacctcccgcaccggctgcaagcgaaacgacgactctcaattccctgtcttcactccgccctctggtggaggcaccaacccctctactccgacaacccctccctcttcaggcggcggttccggatgtacggcggataaatacgctcagtgtggcggctcggggtggtctggctgcaccaactgcccgtctggatcgacctgcaagactatcaacgattggtaccaccagtgtgcctaa

SEQ ID NO: 20 - protein, 293 aa, Artificial Sequence
  Synthetic Construct
MRSSTVLQTGLVAALPFAVQAASGSGQSTRYWDCCKPSCSWSGKASVNRPVLACDANNNPLSDASVKSGCDGGSAYTCANNSPWAVNDQLSYGFAATKLSGGTESSWCCACYALTFTSGPVAGKTMVVQSTSTGGDLGSNHFDINMPGGGVGLFDGCTRQFGGLPGAQYGGISSRSQCDSFPAALKPGCQWRFDWFQNADNPNFTFKQVQCPSELTSRTGCKRNDDSQFPVFTPPSGGGTNPSTPTTPPSSGGGSGCTADKYAQCGGSGWSGCTNCPSGSTCKTINDWYHQCA

SEQ ID NO: 21 - DNA, 1104 bp, Artificial Sequence
  synthetic construct
  sig_peptide (1)..(63)
  exon (1)..(89)
  CDS (1)..(89)
  mat_peptide (64)..(1101)
  Intron (90)..(167)
  exon (168)..(416)
  CDS (168)..(416)
  Intron (417)..(485)
  exon (486)..(1101)
  CDS (486)..(1101)
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaagcgcaacgacgatggtaacttccctgtgttcacccctccttcgggaggccagtcctcctcgtcctcctcgtcctcgtcggccaagccgacatcgacgtcgacgtccactacttcgaccaaagcaacctccacaacttccaccgcctcgtcccagacgtcgtcgtccaccggaggaggctgcgcagcgcagaagtacgctcagtgcggtggcatcggcttcagcggatgcaccacctgtgtctctggcaccacctgcaacaagcagaacgactactactcgcagtgcctctga

SEQ ID NO: 22 - protein, 318 aa, Artificial Sequence
  Synthetic Construct
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDGNFPVFTPPSGGQSSSSSSSSSAKPTSTSTSTTSTKATSTTSTASSQTSSSTGGGCAAQKYAQCGGIGFSGCTTCVSGTTCNKQNDYYSQCL

SEQ ID NO: 23 - DNA, 1104 bp, Artificial Sequence
  synthetic construct
  sig_peptide (1)..(63)
  exon (1)..(89)
  CDS (1)..(89)
  mat_peptide (64)..(1101)
  Intron (90)..(167)
  exon (168)..(416)
  CDS (168)..(416)
  Intron (417)..(485)
  exon (486)..(1101)
  CDS (486)..(1101)
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaagcgcaacgacgatggtaacttccctgtgttcacccctccttcgggaggccagtcctcctcgtcctcctcgtcctcgtcggccaagccgacatcgacgtcgacgtccactacttcgaccaaagcaacctccacaacttccaccgcctcgtcccagacgtcgtcgtccaccggaggaggctgcgcagcgcagcgttacgctcagtgcggtggcatcggctacagcggatgcaccacctgtgtctctggcaccacctgcaacaagcagaacgactactactcgcagtgcctctga

SEQ ID NO: 24 - protein, 318 aa, Artificial Sequence
  Synthetic Construct
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDGNFPVFTPPSGGQSSSSSSSSSAKPTSTSTSTTSTKATSTTSTASSQTSSSTGGGCAAQRYAQCGGIGYSGCTTCVSGTTCNKQNDYYSQCL

SEQ ID NO: 25 - DNA, 1104 bp, Artificial Sequence
  synthetic construct
  sig_peptide (1)..(63)
  exon (1)..(89)
  CDS (1)..(89)
  mat_peptide (64)..(1101)
  Intron (90)..(167)
  exon (168)..(416)
  CDS (168)..(416)
  Intron (417)..(485)
  exon (486)..(1101)
  CDS (486)..(1101)
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaagcgcaacgacgatggtaacttccctgtgttcacccctccttcgggaggccagtcctcctcgtcctcctcgtcctcgtcggccaagccgacatcgacgtcgacgtccaacacttcgaccaaagcaacctccacaaactccaccgcctcgtcccagacgtcgtcgtccaccggaggaggctgcgcagcgcagcgttgggctcagtgcggtggcatcggcttcagcggatgcaccacctgtgtctctggcaccacctgcaacaagcagaacgactactactcgcagtgcctctga

SEQ ID NO: 26 - protein, 318 aa, Artificial Sequence
  Synthetic Construct
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDGNFPVFTPPSGGQSSSSSSSSSAKPTSTSTSNTSTKATSTNSTASSQTSSSTGGGCAAQRWAQCGGIGFSGCTTCVSGTTCNKQNDYYSQCL

SEQ ID NO: 27 - DNA, 1104 bp, Artificial Sequence
  synthetic construct
  sig_peptide (1)..(63)
  exon (1)..(89)
  CDS (1)..(89)
  mat_peptide (64)..(1101)
  Intron (90)..(167)
  exon (168)..(416)
  CDS (168)..(416)
  Intron (417)..(485)
  exon (486)..(1101)
  CDS (486)..(1101)
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaagcgcaacgacgatggtaacttccctgtgttcacccctccttcgggaggccagtcctcctcgtcctcctcgtcctcgtcggccaagccgacatcgacgtcgacgtccactacttcgaccaaagcaacctccacaacttccaccgcctcgtcccagacgtcgtcgtccaccggaggaggctgcgcagcgcagcgttgggctcagtgcggtggcatcggctacagcggatgcaccacctgtgtctctggcaccacctgcaacaagcagaacgactactactcgcagtgcctctga

SEQ ID NO: 28 - protein, 318 aa, Artificial Sequence
  Synthetic Construct
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDGNFPVFTPPSGGQSSSSSSSSSAKPTSTSTSTTSTKATSTTSTASSQTSSSTGGGCAAQRWAQCGGIGYSGCTTCVSGTTCNKQNDYYSQCL

SEQ ID NO: 29 - DNA, 1104 bp, Artificial Sequence
  synthetic construct
  sig_peptide (1)..(63)
  exon (1)..(89)
  CDS (1)..(89)
  mat_peptide (64)..(1101)
  Intron (90)..(167)
  exon (168)..(416)
  CDS (168)..(416)
  Intron (417)..(485)
  exon (486)..(1101)
  CDS (486)..(1101)
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaagcgcaacgacgatggtaacttccctgtgttcacccctccttcgggaggccagtcctcctcgtcctcctcgtcctcgtcggccaagccgacatcgacgtcgacgtccactacttcgaccaaagcaacctccacaacttccaccgcctcgtcccagacgtcgccgtccaccggaggaggctgcgcagcgcagcgttgggctcagtgcggtggcatcggcttcagcggatgcaccacctgtgtctctggcaccacctgcaacaagcagaacgactactactcgcagtgcctctga

SEQ ID NO: 30 - protein, 318 aa, Artificial Sequence
  Synthetic Construct
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDGNFPVFTPPSGGQSSSSSSSSSAKPTSTSTSTTSTKATSTTSTASSQTSPSTGGGCAAQRWAQCGGIGFSGCTTCVSGTTCNKQNDYYSQCL

SEQ ID NO: 31 - DNA, 1104 bp, Artificial Sequence
  synthetic construct
  sig_peptide (1)..(63)
  exon (1)..(89)
  CDS (1)..(89)
  mat_peptide (64)..(1101)
  Intron (90)..(167)
  exon (168)..(416)
  CDS (168)..(416)
  Intron (417)..(485)
  exon (486)..(1101)
  CDS (486)..(1101)
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaagcgcaacgacgatggtaacttccctgtgttcacccctccttcgggaggccagtcctcctcgtcctcctcgtcctcgtcggccaagccgacatcgacgtcgacgtccactacttcgaccaaagcaacctccacaacttccaccgcctcgtcccagacgtcgtcgtccaccggaggaggctgcgcagcgcagcgttacgctcagtgcggtggcatcggcttcagcggatgcaccacctgtgtctctggcaccacctgcaacaagcagaacgactactactcgcagtgcctctga

SEQ ID NO: 32 - protein, 318 aa, Artificial Sequence
  Synthetic Construct
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDGNFPVFTPPSGGQSSSSSSSSSAKPTSTSTSTTSTKATSTTSTASSQTSSSTGGGCAAQRYAQCGGIGFSGCTTCVSGTTCNKQNDYYSQCL

SEQ ID NO: 33 - DNA, 1104 bp, Artificial Sequence
  synthetic construct
  sig_peptide (1)..(63)
  exon (1)..(89)
  CDS (1)..(89)
  mat_peptide (64)..(1101)
  Intron (90)..(167)
  exon (168)..(416)
  CDS (168)..(416)
  Intron (417)..(485)
  exon (486)..(1101)
  CDS (486)..(1101)
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaagcgcaacgacgatggtaacttccctgtgttcacccctccttcgggaggccagtcctcctcgtcctcctcgtcctcgtcggccaagccgacatcgacgtcgacgtccactacttcgaccaaagcaacctccacaacttccaccgcctcgtcccagacgtcgtcgtccaccggaggaggctgcgcagcgcagaagtgggctcagtgcggtggcatcggcttcagcggatgcaccacctgtgtctctggcaccacctgcaacaagcagaacgactactactcgcagtgcctctga

SEQ ID NO: 34 - protein, 318 aa, Artificial Sequence
  Synthetic Construct
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDGNFPVFTPPSGGQSSSSSSSSSAKPTSTSTSTTSTKATSTTSTASSQTSSSTGGGCAAQKWAQCGGIGFSGCTTCVSGTTCNKQNDYYSQCL

SEQ ID NO: 35 - DNA, 1104 bp, Artificial Sequence
  synthetic construct
  sig_peptide (1)..(63)
  exon (1)..(89)
  CDS (1)..(89)
  mat_peptide (64)..(1101)
  Intron (90)..(167)
  exon (168)..(416)
  CDS (168)..(416)
  Intron (417)..(485)
  exon (486)..(1101)
  CDS (486)..(1101)
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaagcgcaacgacgatggtaacttccctgtgttcacccctccttcgggaggccagtcctccccgtcctccccgtcctcgtcggccaagccgacatcgacgtcgacgtccactacttcgaccaaagcaacctccacaacttccaccgcctcgtcccagacgtcgtcgtccaccggaggaggctgcgcagcgcagcgttgggctcagtgcggtggcatcggcttcagcggatgcaccacctgtgtctctggcaccacctgcaacaagcagaacgactactactcgcagtgcctctga

SEQ ID NO: 36 - protein, 318 aa, Artificial Sequence
  Synthetic Construct
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDGNFPVFTPPSGGQSSPSSPSSSAKPTSTSTSTTSTKATSTTSTASSQTSSSTGGGCAAQRWAQCGGIGFSGCTTCVSGTTCNKQNDYYSQCL

SEQ ID NO: 37 - DNA, 1044 bp, Thielavia terrestris
  Intron (90)..(167)
  Intron (417)..(485)
atgcgctctactcccgttcttcgcacaaccctggccgctgcacttcctctggtcgcctccgcggccagtggcagtggccagtccacgaggtatgcgtccctcaatgcgaatgcctcaccaaacgagctaggtgtccaggacgccgcccatgctgactcgtttcccagatactgggactgctgcaagccgtcgtgcgcttggcccgggaaggccgccgtcagccaaccggtctacgcgtgcgatgccaacttccagcgcctgtccgacttcaatgtccagtcgggctgcaacggcggctcggcctactcctgcgccgaccagactccctgggcggtgaacgacaatctcgcctacggcttcgccgcgacgagcatcgccggcgggtccgaatcctcgtggtgctgcgcctgctacgcgtaagtcctctctgccagctaccaggaagttggattagcgcgagctaacctcactccatcacactccaggctcaccttcacttccggtcccgtcgccggcaagacaatggtggtgcagtcaacgagcactggcggcgacctgggaagtaaccatttcgatatcgccatgcccggcggcggcgtgggcatcttcaacggctgcagctcgcagttcggcggcctccccggcgctcaatacggcggcatttcgtcgcgcgaccagtgcgattccttccccgcgccgctcaagcccggctgccagtggcggtttgactggttccagaacgccgacaacccgacgttcacgttccagcaggtgcagtgccccgccgagatcgttgcccgctccggctgcaagcgcaacgacgactccagcttccccgtcttcacccccccaagcggtggcaacggtggcaccgggacgcccacgtcgactgcgcctgggtcgggccagacgtctcccggcggcggcagtggctgcacgtctcagaagtgggctcagtgcggtggcatcggcttcagcggatgcaccacctgtgtctctggcaccacctgccagaagttgaacgactactactcgcagtgcctc

SEQ ID NO: 38 - DNA, 951 bp, Staphylotrichum coccosporum
atgcgctcctcccctgtcctcaggacagcgctcgcagcagccctccccctcgcagcgttggcagcagatggaaagtcgacacgctattgggactgttgtaagccttcgtgttcgtggcctggcaaggcatccgtgaaccagccggtcttcgcatgttccgcaaacttccagcggatctccgatccgaacgtcaaatccggttgtgatggaggctccgcctacgcgtgtgccgaccagacaccttgggcagtcaacgacaacttctcctacggcttcgcagcaacttcgatctcgggtggcaacgaggcctcgtggtgttgtggatgttacgaactcaccttcacttcgggacctgtggcaggtaagaccatggtcgtccagtcgacgtcgacaggaggcgacctcggtaccaaccacttcgatttggcgatgcctggaggaggcgtgggcatcttcgacggctgttcgcctcagttcggaggcttggcaggcgaccggtatggcggtgtgtcctcccggtcgcagtgtgattccttccctgcagccctcaaacccggttgttactggcgattcgactggttcaagaacgccgacaaccctaccttcacgttccgacaggtgcagtgtccgtcggagttggtcgccaggaccggctgtcgcaggaacgatgatggtaacttccctgtgttcacccctccttcgggaggccagtcctcctcgtcctcctcgtcctcgtcggccaagccgacatcgacgtcgacgtccactacttcgaccaaagcaacctccacaacttccaccgcctcgtcccagacgtcgtcgtccaccggaggaggctgtgcagcgcagcgttgggcacagtgtggtggtatcggattctcgggatgtactacatgtgtgtcgggcacgacgtgtaacaagcagaacgactggtactcccagtgtctctaa

SEQ ID NO: 39 - DNA, 935 bp, Neurospora tetrasperma
atgcgctcctccactgttctgcaaaccgggctagtggccgctcttcccttcgccgttcaggccgcgtccggatccggccagtccaccagatattgggactgctgcaaaccatcttgctcctggtccggcaaggcttctgtcaaccgacccgtcctcgcttgcgatgcaaacaacaaccccctgagcgacgccagtgtcaagtctggatgtgacggcggttctgcatacacctgtgccaacaactcaccatgggcggtgaacgaccagctctcctacggctttgctgccacgaaactcagtggtggaaccgagtcgtcttggtgctgtgcctgttatgcgtgagtttcgcgtcaacagctgggtatcacttgatggctaatttatactgcagccttaccttcacttcgggtcctgttgctggcaagaccatggtcgttcagtctaccagtaccggcggtgatctcggctccaaccacttcgatatcaacatgcccggcggcggcgtcggcctgtttgatggctgtacacgacagtttggcggtctccccggcgctcaatatggcggcatcagctcccgcagccagtgcgattcattccctgccgcgctcaagcccggttgccagtggcgcttcgactggttccagaacgccgacaaccccaacttcaccttcaagcaggtccaatgcccatccgagctcacctcccgcaccggctgcaagcgaaacgacgactctcaattccctgtcttcactccgccctctggtggaggcaccaacccctctactccgacaacccctccctcttcaggcggcggttccggatgtacggcggataaatacgctcagtgtggcggctcggggtggtctggctgcaccaactgcccgtctggatcgacctgcaagactatcaacgattattaccaccagtgtgcctaa

SEQ ID NO: 40 - DNA, 279 bp, Artificial Sequence
  synthetic construct
gatggtaacttccctgtgttcacccctccttcgggaggccagtcctcctcgtcctcctcgtcctcgtcggccaagccgacatcgacgtcgacgtccactacttcgaccaaagcaacctccacaacttccaccgcctcgtcccagacgtcgtcgtccaccggaggaggctgtgcagcgcagcgttgggcacagtgtggtggtatcggattctcgggatgtactacatgtgtgtcgggcacgacgtgtaacaagcagaacgactggtactcccagtgtctc

SEQ ID NO: 41 - DNA, 41 bp, Artificial Sequence
  synthetic construct
gctgcaagcgcaacgacgatggtaacttccctgtgttcacc

SEQ ID NO: 42 - DNA, 41 bp, Artificial Sequence
  synthetic construct
ggtgaacacagggaagttaccatcgtcgttgcgcttgcagc

SEQ ID NO: 43 - DNA, 44 bp, Artificial Sequence
  synthetic construct
ccctctagatctcgagtcagagacactgggagtaccagtcgttc

SEQ ID NO: 44 - DNA, 37 bp, Artificial Sequence
  synthetic construct
cgtcgtccaccggaggaggctgcacgtctcagaagtg

SEQ ID NO: 45 - DNA, 37 bp, Artificial Sequence
  synthetic construct
cacttctgagacgtgcagcctcctccggtggacgacg

SEQ ID NO: 46 - DNA, 36 bp, Artificial Sequence
  synthetic construct
ccctctagatctcgagggaaatcaaccagcagtcgc

SEQ ID NO: 47 - DNA, 34 bp, Artificial Sequence
  synthetic construct
cgtcgtccaccggaggaggctgcgcagcgcagcg

SEQ ID NO: 48 - DNA, 17 bp, Artificial Sequence
  synthetic construct
tcctccggtggacgacg

SEQ ID NO: 49 - DNA, 41 bp, Artificial Sequence
  synthetic construct
ccctctagatctcgagtcagaggcactgcgagtaccagtcg

SEQ ID NO: 50 - DNA, 41 bp, Artificial Sequence
  synthetic construct
acacaactggggatccaccatgcgctctactcccgttcttc

SEQ ID NO: 51 - DNA, 40 bp, Artificial Sequence
  synthetic construct
ccctctagatctcgagtcagaggcactgcgagtagtagtc

SEQ ID NO: 52 - DNA, 30 bp, Artificial Sequence
  synthetic construct
agctggagtcgtcgttcgccttgcagccgg

SEQ ID NO: 53 - DNA, 36 bp, Artificial Sequence
  synthetic construct
ctccggctgcaaggcgaacgacgactccagcttccc

SEQ ID NO: 54 - DNA, 41 bp, Artificial Sequence
  synthetic construct
ccctctagatctcgagttagaggcactgcgagtagtagtcg

SEQ ID NO: 55 - DNA, 40 bp, Artificial Sequence
  synthetic construct
ccctctagatctcgagttagaggcactgcgagtaccagtc

SEQ ID NO: 56 - DNA, 35 bp, Artificial Sequence
  synthetic construct
acacaactggggatccaccatgcgctcctcccctg

SEQ ID NO: 57 - DNA, 41 bp, Artificial Sequence
  synthetic construct
ccctctagatctcgagttagagacactgggagtaccagtcg

SEQ ID NO: 58 - DNA, 44 bp, Artificial Sequence
  synthetic construct
ccctctagatctcgagttagagacactgggagtagtagtcgttc

SEQ ID NO: 59 - DNA, 32 bp, Artificial Sequence
  synthetic construct
tggagtcgtcgtttgcgcgacagccggtcctg

SEQ ID NO: 60 - DNA, 36 bp, Artificial Sequence
  synthetic construct
gaccggctgtcgcgcaaacgacgactccagcttccc

SEQ ID NO: 61 - DNA, 38 bp, Artificial Sequence
  synthetic construct
acacaactggggatccaccatgcgctcctccactgttc

SEQ ID NO: 62 - DNA, 40 bp, Artificial Sequence
  synthetic construct
ccctctagatctcgagttaggcacactggtggtaccaatc

SEQ ID NO: 63 - DNA, 30 bp, Artificial Sequence
  synthetic construct
cgcactgagcgtacttctgcgctgcgcagc

SEQ ID NO: 64 - DNA, 20 bp, Artificial Sequence
  synthetic construct
aagtacgctcagtgcggtgg

SEQ ID NO: 65 - DNA, 34 bp, Artificial Sequence
  synthetic construct
gctgtagccgatgccaccgcactgagcgtaacgc

SEQ ID NO: 66 - DNA, 34 bp, Artificial Sequence
  synthetic construct
gcgttacgctcagtgcggtggcatcggctacagc

SEQ ID NO: 67 - DNA, 37 bp, Artificial Sequence
  synthetic construct
ggagtttgtggaggttgctttggtcgaagtgttggac

SEQ ID NO: 68 - DNA, 38 bp, Artificial Sequence
  synthetic construct
cgtccaacacttcgaccaaagcaacctccacaaactcc

SEQ ID NO: 69 - DNA, 32 bp, Artificial Sequence
  synthetic construct
gctgtagccgatgccaccgcactgagcccaac

SEQ ID NO: 70 - DNA, 34 bp, Artificial Sequence
  synthetic construct
gcgttgggctcagtgcggtggcatcggctacagc

SEQ ID NO: 71 - DNA, 35 bp, Artificial Sequence
  synthetic construct
ggacggcgacgtctgggacgaggcggtggaagttg

SEQ ID NO: 72 - DNA, 32 bp, Artificial Sequence
  synthetic construct
cttccaccgcctcgtcccagacgtcgccgtcc

SEQ ID NO: 73 - DNA, 31 bp, Artificial Sequence
  synthetic construct
agcgtaacgctgcgctgcgcagcctcctccg

SEQ ID NO: 74 - DNA, 33 bp, Artificial Sequence
  synthetic construct
ccggaggaggctgcgcagcgcagcgttacgctc

SEQ ID NO: 75 - DNA, 32 bp, Artificial Sequence
  synthetic construct
ccacttctgcgctgcgcagcctcctccggtgg

SEQ ID NO: 76 - DNA, 32 bp, Artificial Sequence
  synthetic construct
ccaccggaggaggctgcgcagcgcagaagtgg

SEQ ID NO: 77 - DNA, 32 bp, Artificial Sequence
  synthetic construct
ggacggggaggacggggaggactggcctcccg

SEQ ID NO: 78 - DNA, 31 bp, Artificial Sequence
  synthetic construct
gggaggccagtcctccccgtcctccccgtcc

SEQ ID NO: 79 - DNA, 114 bp, Artificial Sequence
  synthetic construct
ggctgcgcagcgcagcgttgggctcagtgcggtggcatcggcttcagcggatgcaccacctgtgtctctggcaccacctgcaacaagcagaacgactggtactcgcagtgcctc

(19) United States
(12) Patent Application Publication
(10) Pub. No.: US 2019/0106690 A1
(43) Pub. Date: Apr. 11, 2019

(54) Cellulase Variants And Polynucleotides Encoding Same

(71) Applicant: Novozymes A/S, Bagsvaerd (DK)

(72) Inventors: Weijian Lai, Beijing (CN); Wei Peng, Beijing (CN); Ye Liu, Beijing (CN); Ming Li, Beijing (CN)

(73) Assignee: Novozymes A/S, Bagsvaerd (DK)

(21) Appl. No.: 15/775,120

(22) PCT Filed: Nov. 15, 2016
(86) PCT No.: PCT/CN2016/105927

§ 371 Date: May 10, 2018

(30) Foreign Application Priority Data
Nov. 16, 2015 (CN) PCT/CN2015/094695

(51) Int. Cl. C12N 9/42; D06M 16/00

(52) U.S. Cl. CPC C12N 9/2437; D06M 2101/06; D06M 16/003; C12Y 302/01004

(57) ABSTRACT

Provided are cellulase variants and polynucleotides encoding the variants. And provided are nucleic acid constructs, vectors, and host cells comprising the polynucleotides. Furthermore provided are methods of using the variants.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to cellulase variants, polynucleotides encoding the variants, methods of producing the variants, and methods of using the variants.

### Description of the Related Art

Cellulase enzymes are widely used to improve the appearance and softness of cellulose-containing fabrics. A widespread application of cellulase enzymes is to remove cotton fuzz and loose surface fibers in or on the fabric. This process is referred to as “biopolishing” and smoothes the surface of the fabric, which in turn improves its softness and appearance. Cellulase treatment also aids in the prevention of subsequent formation of fiber pills that make the garments appear worn. During depilling it is desirable to minimize strength loss of the fabric due to the hydrolytic action of the cellulases.

Another industrial application of cellulase enzymes is for treating denim fabrics so as to impart to them a “stone-washed” appearance. Such a process is known in the industry as “biostoning”. The term biostoning was adopted as pumice stones were traditionally used to treat the fabric. However, cellulases have largely replaced pumice stones in recent years. Biostoning is quite different from depilling in that biostoning aims to remove colour from denim and control its re-deposition on the fabric while depilling aims to solely improve softness and appearance as in depilling.

Cellulase enzymes are a group of glycoside hydrolase enzymes that catalyze the hydrolysis of beta-1,4-glycosidic linkages in the cellulose polymer and often comprise a cellulose binding domain (CBD) and a catalytic domain. A region between these two domains known as a “linker” or “linker peptide” serves as a flexible spacer between the CBD and the catalytic domain. The catalytic domains of individual cellulase components are classified by both the Enzyme Commission (EC) and the Glycoside Hydrolase (GH) family systems. The Enzyme Commission distinguishes two classes of cellulases based on their preference for cleavage of internal beta-1,4 linkages (endoglucanase or “EG”, EC 3.2.1.4) or the release of cellobiose from the reducing or non-reducing end of the cellulose polymer (cellobiohydrolases or “CBH”, EC 3.2.1. 91, sometimes also referred to as exoglucanases). In contrast, the GH family system distinguishes the catalytic domains of cellulase components based on the conservation of primary and secondary structure, as well as the stereochemistry of the catalytic reaction. The GH family designations for all known cellulase catalytic and binding domains is provided and continually updated through the Carbohydrate-Active EnZymes (CAZy) database (Cantarel et al, 2009, Nucleic Acids Res 37:D233-238) available at the URL: cazy.org. Cellulase enzymes may be found in a number of GH Families including, but not limited to, Families 5, 6, 7, 8, 9, 10, 12, 16, 18, 19, 26, 44, 45, 48, 51, 61 and 74. Further, cellulase in some of the larger GH Families may be grouped into subfamilies.

Industrially well-performing endo-β-1,4-glucanases are described in e.g. WO 91/17243, WO 91/17244 and WO 91/10732. Specific cellulase variants are described in WO 94/07998 and WO 1998/012307. U.S. Pat. No. 7,981,654 discloses a cellulase fusion protein comprising A. an optionally modified first amino acid sequence of a cellulase core derived from one species, and B. an optionally modified second amino acid sequence of a linker and/or cellulose binding domain (CBD) derived from another species, wherein a junction region has been introduced between said first amino acid sequence and said second amino acid sequence.

Despite these efforts, there is still a need for improved combinations of cellulase enzymes and compositions thereof that are more effective in biofinishing cellulose-containing textile. In particular, there is a continuous need for more efficient cellulase enzyme composition to improve the process economics. The present invention aims to meet these needs.

The present invention provides cellulase variants with improved properties compared to its parent.

### SUMMARY OF THE INVENTION

The present invention relates to cellulase variants, comprising an alteration at one or more (e.g., several) positions corresponding to positions 292, 274, 266, 265, 255, 246, 237, 224 and 221 of the mature polypeptide of SEQ ID NO: 2, wherein the variants have cellulase activity.

The present invention further relates to variants of a parent GH45 cellulase, comprising a catalytic domain, and a cellulose binding domain, wherein the cellulase binding domain is heterologous to the catalytic domain, and wherein the variant has an improved biofinishing activity compared with the parent GH45 cellulase.

The present invention also relates to polynucleotides encoding the variants; nucleic acid constructs, vectors, and host cells comprising the polynucleotides; and methods of producing the variants.

The present invention also relates to methods of treating cellulose-containing textile.

### DEFINITIONS

cellulase: The term “cellulase” means a group of glycoside hydrolase enzymes that catalyze the hydrolysis of beta-1,4-glycosidic linkages in the cellulose polymer. For purposes of the present invention, cellulase activity is determined according to the procedure described in the Examples. In one aspect, the variants of the present invention have at least 20%, e.g., at least 40%, at least 50%, at least 60%, at least 70%, at least 80%, at least 90%, at least 95%, or at least 100% of the cellulase activity of the mature polypeptide of SEQ ID NO: 2.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of a variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Fragment: The term “fragment” means a polypeptide having one or more (e.g., several) amino acids absent from the amino and/or carboxyl terminus of a mature polypeptide; wherein the fragment has cellulase activity. In one aspect, a fragment contains at least 200 amino acid residues (e.g., amino acids 200 to 315, e.g., 210 to 290, 215 to 295, 220 to 300 of SEQ ID NO: 2.

High stringency conditions: The term “high stringency conditions” means for probes of at least 100 nucleotides in length, prehybridization and hybridization at 42° C. in 5X SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2X SSC, 0.2% SDS at 65° C.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Improved property: The term “improved property” means a characteristic associated with a variant that is improved compared to the parent. Such improved properties include, but are not limited to, an improved biofinishing property, a reduced weight loss of cellulose-containing textile, and a reduced loss in cellulose-containing textile strength.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids -21 to -1 of SEQ ID NO: 2 based on the program SignalP (Nielsen et al., 1997, Protein Engineering 10: 1-6) that predicts amino acids -21 to -1 of SEQ ID NO: 2 are a signal peptide. It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide having cellulase activity. In one aspect, the mature polypeptide coding sequence is nucleotides 1 to 63 of SEQ ID NO: 1 or the cDNA sequence thereof based on the program SignalP (Nielsen et al., 1997, supra) that predicts nucleotides 1 to 63 of SEQ ID NO: 1 encode a signal peptide.

Medium stringency conditions: The term “medium stringency conditions” means for probes of at least 100 nucleotides in length, prehybridization and hybridization at 42° C. in 5X SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2X SSC, 0.2% SDS at 55° C.

Medium-high stringency conditions: The term “medium-high stringency conditions” means for probes of at least 100 nucleotides in length, prehybridization and hybridization at 42° C. in 5X SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2X SSC, 0.2% SDS at 60° C.

Mutant: The term “mutant” means a polynucleotide encoding a variant.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent or parent cellulase: The term “parent” or “parent cellulase” means any polypeptide with cellulase activity to which an alteration is made to produce the enzyme variants of the present invention.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the —nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the —nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence; wherein the subsequence encodes a fragment having cellulase activity. In one aspect, a subsequence contains nucleotides 600 to 945 of SEQ ID NO: 1 (e.g., nucleotides 645 to 885, nucleotides 660 to 900 of SEQ ID NO: 1).

Variant: The term “variant” means a polypeptide having cellulase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. The term “variant” can also means a “hybrid polypeptide” in which a region of one polypeptide is fused at the N-terminus or the C-terminus of a region of another polypeptide or a “fusion polypeptide” in which another polypeptide is fused at the N-terminus or the C-terminus of a polypeptide. The variants of the present invention have at least 20%, e.g., at least 40%, at least 50%, at least 60%, at least 70%, at least 80%, at least 90%, at least 95%, or at least 100%, at least 110%, at least 120%, at least 150%, at least 180%, at least 200% of the cellulase activity of the mature polypeptide of the parent cellulase.

Very high stringency conditions: The term “very high stringency conditions” means for probes of at least 100 nucleotides in length, prehybridization and hybridization at 42° C. in 5X SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2X SSC, 0.2% SDS at 70° C.

Very low stringency conditions: The term “very low stringency conditions” means for probes of at least 100 nucleotides in length, prehybridization and hybridization at 42° C. in 5X SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2X SSC, 0.2% SDS at 45° C.

Wild-type cellulase: The term “wild-type” cellulase means a cellulase expressed by a naturally occurring microorganism, such as a bacterium, yeast, or filamentous fungus found in nature.

### Conventions for Designation of Variants

For purposes of the present invention, the mature polypeptide disclosed in SEQ ID NO: 2 is used to determine the corresponding amino acid residue in another cellulase. The amino acid sequence of another cellulase is aligned with the mature polypeptide disclosed in SEQ ID NO: 2, and based on the alignment, the amino acid position number corresponding to any amino acid residue in the mature polypeptide disclosed in SEQ ID NO: 2 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another cellulase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537:39-64; Katoh and Toh, 2010, Bioinformatics 26:_1899-1900), and EMBOSS EMMA employing ClustalW (1.83 or later; Thompson et al., 1994, Nucleic Acids Research 22: 4673-4680), using their respective default parameters.

When the other enzyme has diverged from the mature polypeptide of SEQ ID NO: 2 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Glyl95*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations. Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

### “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The present invention relates to cellulase variants, comprising an alteration at one or more positions corresponding to positions 292, 274, 266, 265, 255, 246, 237, 224 and 221 of the mature polypeptide of SEQ ID NO: 2, and the variant has cellulase activity .

The present invention relates to variants of a parent GH45 cellulase, comprising a catalytic domain, and a cellulose binding domain, wherein the cellulase binding domain is heterologous to the catalytic domain, and wherein the cellulase variant has an improved biofinishing activity compared with the parent GH45 cellulase.

### Variants

The present invention provides cellulase variants, comprising an alteration at one or more (e.g., several) positions corresponding to positions 292, 274, 266, 265, 255, 246, 237, 224 and 221, wherein each alteration is independently a substitution, insertion or deletion and the variant has cellulase activity.

In one aspect, the cellulase is GH45 cellulase.

In another aspect, the alteration is a substitution.

In another aspect, the variant is a variant of a parent cellulase, comprising a catalytic domain and a cellulase binding domain, wherein the cellulase binding domain is heterologous to the catalytic domain. In a preferable embodiment, a linker is between the catalytic domain and the cellulose binding domain. In a more preferable embodiment, the linker is homologous to the cellulose binding domain. In a more preferable embodiment, the linker is homologous to the catalytic domain.

In another aspect, the variant has sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent cellulase.

In one aspect, the number of alterations in the variants of the present invention is 1-20, e.g., 1-10 and 1-5, such as 1, 2, 3, 4, 5, 6, 7, 8, 9 or 10 alterations.

In another aspect, a variant comprises an alteration at one or more (e.g., several) positions corresponding to positions 292, 274, 266, 265, 255, 246, 237, 224 and 221. In another aspect, a variant comprises an alteration at two positions corresponding to any of positions 292, 274, 266, 265, 255, 246, 237, 224 and 221. In another aspect, a variant comprises an alteration at three positions corresponding to any of positions 292, 274, 266, 265, 255, 246, 237, 224 and 221. In another aspect, a variant comprises an alteration at each position corresponding to positions 292, 274, 266, 265, 255, 246, 237, 224 and 221.

In another aspect, the variant comprises or consists of an alteration at a position corresponding to position 292. In another aspect, the amino acid at a position corresponding to position 292 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr. In another aspect, the variant comprises or consists of the substitution W292Y of the mature polypeptide of SEQ ID NO: 2.

In another aspect, the variant comprises or consists of an alteration at a position corresponding to position 274. In another aspect, the amino acid at a position corresponding to position 274 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr. In another aspect, the variant comprises or consists of the substitution F274Y of the mature polypeptide of SEQ ID NO: 2.

In another aspect, the variant comprises or consists of an alteration at a position corresponding to position 266. In another aspect, the amino acid at a position corresponding to position 266 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr. In another aspect, the variant comprises or consists of the substitution W266Y of the mature polypeptide of SEQ ID NO: 2.

In another aspect, the variant comprises or consists of an alteration at a position corresponding to position 265. In another aspect, the amino acid at a position corresponding to position 265 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys. In another aspect, the variant comprises or consists of the substitution R265K of the mature polypeptide of SEQ ID NO: 2.

In another aspect, the variant comprises or consists of an alteration at a position corresponding to position 255. In another aspect, the amino acid at a position corresponding to position 255 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro. In another aspect, the variant comprises or consists of the substitution S255P of the mature polypeptide of SEQ ID NO: 2.

In another aspect, the variant comprises or consists of an alteration at a position corresponding to position 246. In another aspect, the amino acid at a position corresponding to position 246 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asn. In another aspect, the variant comprises or consists of the substitution T246N of the mature polypeptide of SEQ ID NO: 2.

In another aspect, the variant comprises or consists of an alteration at a position corresponding to position 237. In another aspect, the amino acid at a position corresponding to position 237 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asn. In another aspect, the variant comprises or consists of the substitution T237N of the mature polypeptide of SEQ ID NO: 2.

In another aspect, the variant comprises or consists of an alteration at a position corresponding to position 224. In another aspect, the amino acid at a position corresponding to position 224 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro. In another aspect, the variant comprises or consists of the substitution S224P of the mature polypeptide of SEQ ID NO: 2.

In another aspect, the variant comprises or consists of an alteration at a position corresponding to position 221. In another aspect, the amino acid at a position corresponding to position 221 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro. In another aspect, the variant comprises or consists of the substitution S221P of the mature polypeptide of SEQ ID NO: 2.

In another aspect, the variant comprises or consists of an alteration at positions corresponding to positions 292 and 274, positions 292 and 266, positions 292 and 265, positions 292 and 265, positions 292 and 246, positions 292 and 237, positions 292 and 224, positions 292 and 221, such as those described above.

In another aspect, the variant comprises or consists of alterations at positions corresponding to positions 274 and 266, positions 274 and 265, positions 274 and 255, positions 274 and 246, positions 274 and 237, positions 274 and 224, positions 274 and 221, such as those described above.

In another aspect, the variant comprises or consists of alterations at positions corresponding to positions 266 and 265, positions 266 and 255, positions 266 and 246, positions 266 and 237, positions 266 and 224, positions 266 and 221, such as those described above.

In another aspect, the variant comprises or consists of alterations at positions corresponding to positions 255 and 246, positions 255 and 237, positions 255 and 224, positions 255 and 221, such as those described above.

In another aspect, the variant comprises or consists of alterations at positions corresponding to positions 246 and 237, positions 246 and 224, positions 246 and 221, such as those described above.

In another aspect, the variant comprises or consists of alterations at positions corresponding to positions 237 and 224, positions 237 and 221, such as those described above.

In another aspect, the variant comprises or consists of alterations at positions corresponding to positions 224 and 221, such as those described above.

In another aspect, the variant comprises or consists of alterations at positions corresponding to positions 292, 274 and 266, positions 292, 274 and 265, positions 292, 274 and 255, positions 292, 274 and 246, positions 292, 274 and 237, positions 292, 274 and 224, positions 292, 274 and 221, such as those described above.

In another aspect, the variant comprises or consists of alterations at positions corresponding to positions 292, 266, and 265, positions 292, 266 and 255, positions 292, 266 and 246, positions 292, 266 and 237, positions 292, 266 and 224, positions 292, 266 and 221, such as those described above.

In another aspect, the variant comprises or consists of alterations at positions corresponding to positions 292, 265 and 255, positions 292, 265 and 246, positions 292, 265 and 237, positions 292, 265 and 224, positions 292, 265 and 221, such as those described above.

In another aspect, the variant comprises or consists of alterations at positions corresponding to positions 292, 255 and 246, positions 292, 255 and 237, positions 292, 255 and 224, positions 292, 255 and 221, such as those described above.

In another aspect, the variant comprises or consists of alterations at positions corresponding to positions 292, 246 and 237, positions 292, 246 and 224, positions 292, 246 and 221, such as those described above.

In another aspect, the variant comprises or consists of alterations at positions corresponding to positions 292, 237 and 224, positions 292, 237 and 221, such as those described above.

In another aspect, the variant comprises or consists of alterations at positions corresponding to positions 292, 224 and 221, such as those described above.

In another aspect, the variant comprises or consists of alterations at positions corresponding positions 274, 266 and 265, positions 274, 266 and 255, positions 274, 266 and 246, positions 274, 266 and 237, positions 274, 266 and 224, positions 274, 266 and 221, such as those described above.

In another aspect, the variant comprises or consists of alterations at positions corresponding positions 274, 265 and 255, positions 274, 265 and 246, positions 274, 265 and 237, positions 274, 265 and 224, positions 274, 265 and 221, such as those described above.

In another aspect, the variant comprises or consists of alterations at positions corresponding positions 274, 255 and 246, positions 274, 255 and 237, positions 274, 255 and 224, positions 274, 255 and 221, such as those described above.

In another aspect, the variant comprises or consists of alterations at positions corresponding positions 274, 246 and 237, positions 274, 246 and 224, positions 274, 246 and 221, such as those described above.

In another aspect, the variant comprises or consists of alterations at positions corresponding positions 274, 237 and 224, positions 274, 237 and 221, such as those described above.

In another aspect, the variant comprises or consists of alterations at positions corresponding positions 274, 224 and 221, such as those described above.

In another aspect, the variant comprises or consists of alterations at positions corresponding to positions 266, 265 and 255, positions 266, 265 and 246, positions 266, 265 and 237, positions 266, 265 and 224, positions 266, 265 and 221, such as those described above.

In another aspect, the variant comprises or consists of alterations at positions corresponding to positions 266, 255 and 246, positions 266, 255 and 237, positions 266, 255 and 224, positions 266, 255 and 221, such as those described above.

In another aspect, the variant comprises or consists of alterations at positions corresponding to positions 266, 246 and 237, positions 266, 246 and 224, positions 266, 246 and 221, such as those described above.

In another aspect, the variant comprises or consists of alterations at positions corresponding to positions 266, 237 and 224, positions 266, 237 and 221, such as those described above.

In another aspect, the variant comprises or consists of alterations at positions corresponding to positions 266, 224 and 221, such as those described above.

In another aspect, the variant comprises or consists of alterations at positions corresponding to positions 265, 255 and 246, positions 265, 255 and 237, positions 265, 255 and 224, positions 265, 255 and 221, such as those described above.

In another aspect, the variant comprises or consists of alterations at positions corresponding to positions 265, 246 and 237, positions 265, 246 and 224, positions 265, 246 and 221, such as those described above.

In another aspect, the variant comprises or consists of alterations at positions corresponding to positions 265, 237 and 224, positions 265, 237 and 221, such as those described above.

In another aspect, the variant comprises or consists of alterations at positions corresponding to positions 265, 224 and 221, such as those described above.

In another aspect, the variant comprises or consists of alterations at positions corresponding to positions 255, 246 and 237, positions 255, 246 and 224, positions 255, 246 and 221, such as those described above.

In another aspect, the variant comprises or consists of alterations at positions corresponding to positions 255, 237 and 224, positions 255, 237 and 221, such as those described above.

In another aspect, the variant comprises or consists of alterations at positions corresponding to positions 255, 224 and 221, such as those described above.

In another aspect, the variant comprises or consists of alterations at positions corresponding to positions 246, 237 and 224, positions 246, 237 and 221 such as those described above.

In another aspect, the variant comprises or consists of alterations at positions corresponding to positions 237, 224 and 221, such as those described above.

In another aspect, the variant comprises or consists of alterations at positions corresponding to positions 292, 274, 266 and 265, positions 292, 274, 266 and 255, positions 292, 274, 266 and 246, positions 292, 274, 266 and 237, positions 292, 274, 266 and 224, positions 292, 274, 266 and 221, such as those described above.

In another aspect, the variant comprises or consists of alterations at positions corresponding to positions 292, 266, 265 and 255, positions 292, 266, 265 and 255, positions 292, 266, 265 and 246, positions 292, 266, 265 and 237, positions 292, 266, 265 and 224, positions 292, 266, 265 and 221, such as those described above.

In another aspect, the variant comprises or consists of alterations at positions corresponding to positions 292, 265, 255 and 246, positions 292, 265, 255 and 237, positions 292, 265, 255 and 224, positions 292, 265, 255 and 221, such as those described above.

In another aspect, the variant comprises or consists of alterations at positions corresponding to positions 292, 255, 246 and 237, positions 292, 255, 246 and 224, positions 292, 255, 246 and 221, such as those described above.

In another aspect, the variant comprises or consists of alterations at positions corresponding to positions positions 292, 246, 237 and 224, positions 292, 246, 237 and 221, such as those described above.

In another aspect, the variant comprises or consists of alterations at positions corresponding to positions 292, 237, 224 and 221, such as those described above.

In another aspect, the variant comprises or consists of alterations at positions corresponding positions 274, 266, 265 and 255, positions 274, 266, 265 and 246, positions 274, 266, 265 and 237, positions 274, 266, 265 and 224, positions 274, 266, 265 and 221, such as those described above.

In another aspect, the variant comprises or consists of alterations at positions corresponding positions 274, 265, 255 and 246, positions 274, 265, 255 and 237, positions 274, 265, 255 and 224, positions 274, 265, 255 and 221, such as those described above.

In another aspect, the variant comprises or consists of alterations at positions corresponding positions 274, 255, 246 and 237, positions 274, 255, 246 and 224, positions 274, 255, 246 and 221, such as those described above.

In another aspect, the variant comprises or consists of alterations at positions corresponding positions 274, 246, 237 and 224, positions 274, 246, 237 and 221, such as those described above.

In another aspect, the variant comprises or consists of alterations at positions corresponding positions 274, 237, 224 and 221, such as those described above.

In another aspect, the variant comprises or consists of alterations at positions corresponding to positions 266, 265, 255 and 246, positions 266, 265, 255 and 237, positions 266, 265, 255 and 224, positions 266, 265, 255 and 221, such as those described above.

In another aspect, the variant comprises or consists of alterations at positions corresponding to positions 266, 255, 246 and 237, positions 266, 255, 246 and 224, positions 266, 255, 246 and 221, such as those described above.

In another aspect, the variant comprises or consists of alterations at positions corresponding to positions 266, 246, 237 and 224, positions 266, 246, 237 and 221, such as those described above.

In another aspect, the variant comprises or consists of alterations at positions corresponding to positions 266, 237, 224 and 221, such as those described above.

In another aspect, the variant comprises or consists of alterations at positions corresponding to positions 265, 255, 246 and 237, positions 265, 255, 246 and 224, positions 265, 255, 246 and 221, such as those described above.

In another aspect, the variant comprises or consists of alterations at positions corresponding to positions positions 265, 246, 237 and 224, positions 265, 246, 237 and 221, such as those described above.

In another aspect, the variant comprises or consists of alterations at positions corresponding to positions 265, 237, 224 and 221, such as those described above.

In another aspect, the variant comprises or consists of alterations at positions corresponding to positions 255, 246, 237 and 224, positions 255, 246, 237 and 221, such as those described above.

In another aspect, the variant comprises or consists of alterations at positions corresponding to positions 255, 237, 224 and 221, such as those described above.

In another aspect, the variant comprises or consists of alterations at positions corresponding to positions 246, 237, 224 and 221 such as those described above.

In another aspect, the variant comprises or consists of alterations at positions corresponding to positions 292, 274, 266, 265 and 255, positions 292, 274, 266, 265 and 246, positions 292, 274, 266, 265 and 237, positions 292, 274, 266, 265 and 224, positions 292, 274, 266, 265 and 221, such as those described above.

In another aspect, the variant comprises or consists of alterations at positions corresponding to positions 292, 266, 265, 255 and 246, positions 292, 266, 265, 255 and 237, positions 292, 266, 265, 255 and 224, positions 292, 266, 265, 255 and 221, such as those described above.

In another aspect, the variant comprises or consists of alterations at positions corresponding to positions 292, 265, 255, 246 and 237, positions 292, 265, 255, 246 and 224, positions 292, 265, 255, 246 and 221, such as those described above.

In another aspect, the variant comprises or consists of alterations at positions corresponding to positions 292, 255, 246, 237 and 224, positions 292, 255, 246, 237 and 221, such as those described above.

In another aspect, the variant comprises or consists of alterations at positions corresponding to positions 292, 246, 237, 224 and 221, such as those described above.

In another aspect, the variant comprises or consists of alterations at positions corresponding positions 274, 266, 265, 255 and 246, positions 274, 266, 265, 255 and 237, positions 274, 266, 265, 255 and 224, positions 274, 266, 265, 255 and 221, such as those described above.

In another aspect, the variant comprises or consists of alterations at positions corresponding positions 274, 265, 255, 246 and 237, positions 274, 265, 255, 246 and 224, positions 274, 265, 255, 246 and 221, such as those described above.

In another aspect, the variant comprises or consists of alterations at positions corresponding positions 274, 255, 246, 237 and 224, positions 274, 255, 246, 237 and 221, such as those described above.

In another aspect, the variant comprises or consists of alterations at positions corresponding positions 274, 246, 237, 224 and 221, such as those described above.

In another aspect, the variant comprises or consists of alterations at positions corresponding to positions 266, 265, 255, 246 and 237, positions 266, 265, 255, 246 and 224, positions 266, 265, 255, 246 and 221, such as those described above.

In another aspect, the variant comprises or consists of alterations at positions corresponding to positions 266, 255, 246, 237 and 224, positions 266, 255, 246, 237 and 221, such as those described above.

In another aspect, the variant comprises or consists of alterations at positions corresponding to positions 266, 246, 237, 224 and 221, such as those described above.

In another aspect, the variant comprises or consists of alterations at positions corresponding to positions 265, 255, 246, 237 and 224, positions 265, 255, 246, 237 and 221, such as those described above.

In another aspect, the variant comprises or consists of alterations at positions corresponding to positions 265, 246, 237, 224 and 221, such as those described above.

In another aspect, the variant comprises or consists of alterations at positions corresponding to positions 255, 246, 237, 224 and 221, such as those described above.

In another aspect, the variant comprises or consists of alterations at positions corresponding to positions 292, 274, 266, 265, 255 and 246, positions 292, 274, 266, 265, 255 and 237, positions 292, 274, 266, 265, 255 and 224, positions 292, 274, 266, 265, 255 and 221, such as those described above.

In another aspect, the variant comprises or consists of alterations at positions corresponding to positions 292, 266, 265, 255, 246 and 237, positions 292, 266, 265, 255, 246 and 224, positions 292, 266, 265, 255, 246 and 221, such as those described above.

In another aspect, the variant comprises or consists of alterations at positions corresponding to positions 292, 265, 255, 246, 237 and 224, positions 292, 265, 255, 246, 237 and 221, such as those described above.

In another aspect, the variant comprises or consists of alterations at positions corresponding to positions 292, 255, 246, 237, 224 and 221, such as those described above.

In another aspect, the variant comprises or consists of alterations at positions corresponding positions 274, 266, 265, 255, 246 and 237, positions 274, 266, 265, 255, 246 and 224, positions 274, 266, 265, 255, 246 and 221, such as those described above.

In another aspect, the variant comprises or consists of alterations at positions corresponding positions 274, 265, 255, 246, 237 and 224, positions 274, 265, 255, 246, 237 and 221, such as those described above.

In another aspect, the variant comprises or consists of alterations at positions corresponding positions 274, 255, 246, 237, 224 and 221, such as those described above.

In another aspect, the variant comprises or consists of alterations at positions corresponding to positions 266, 265, 255, 246, 237 and 224, positions 266, 265, 255, 246, 237 and 221, such as those described above.

In another aspect, the variant comprises or consists of alterations at positions corresponding to positions 266, 255, 246, 237, 224 and 221, such as those described above.

In another aspect, the variant comprises or consists of alterations at positions corresponding to positions 265, 255, 246, 237, 224 and 221, such as those described above.

In another aspect, the variant comprises or consists of alterations at positions corresponding to positions 292, 274, 266, 265, 255, 246 and 237, positions 292, 274, 266, 265, 255, 246 and 224, positions 292, 274, 266, 265, 255, 246 and 221, such as those described above.

In another aspect, the variant comprises or consists of alterations at positions corresponding to positions 292, 266, 265, 255, 246, 237 and 224, positions 292, 266, 265, 255, 246, 237 and 221, such as those described above.

In another aspect, the variant comprises or consists of alterations at positions corresponding to positions 292, 265, 255, 246, 237, 224 and 221, such as those described above.

In another aspect, the variant comprises or consists of alterations at positions corresponding positions 274, 266, 265, 255, 246, 237 and 224, positions 274, 266, 265, 255, 246, 237 and 221, such as those described above.

In another aspect, the variant comprises or consists of alterations at positions corresponding positions 274, 265, 255, 246, 237, 224 and 221, such as those described above.

In another aspect, the variant comprises or consists of alterations at positions corresponding to positions 266, 265, 255, 246, 237, 224 and 221, such as those described above.

In another aspect, the variant comprises or consists of alterations at positions corresponding to positions positions 292, 274, 266, 265, 255, 246, 237 and 224, positions 292, 274, 266, 265, 255, 246, 237 and 221, such as those described above.

In another aspect, the variant comprises or consists of alterations at positions corresponding to positions 292, 266, 265, 255, 246, 237, 224 and 221, such as those described above.

In another aspect, the variant comprises or consists of alterations at positions corresponding positions 274, 266, 265, 255, 246, 237, 224 and 221, such as those described above.

In another aspect, the variant comprises or consists of alterations at positions corresponding to positions 292, 274, 266, 265, 255, 246, 237, 224 and 221, such as those described above.

In another aspect, the variant comprises or consists of one or more (e.g., several) substitutions selected from the group consisting of W292Y, F274Y, W266Y, R265K, S255P, T246N, T237N, S224P and S221P.

In another aspect, the variant comprises or consists of the substitutions W292Y+F274Y, W292Y+W266Y, W292Y+R265K, W292Y+S255P, W292Y+T246N, W292Y+T237N, W292Y+S224P, W292Y+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+W266Y, F274Y+R265K, F274Y+S255P, F274Y+T246N, F274Y+T237N, F274Y+S224P, F274Y+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+R265K, W266Y+S255P, W266Y+T246N, W266Y+T237N, W266Y+S224P, W266Y+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions R265K+S255P, R265K+T246N, R265K+T237N, R265K+S224P, R265K+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions S255P+T246N, S255P+T237N, S255P+S224P, S255P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions T246N+T237N, T246N+S224P, T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions T237N+S224P, T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+F274Y +W266Y, W292Y+F274Y+R265K, W292Y+F274Y+S255P, W292Y+F274Y+T246N, W292Y+F274Y+T237N, W292Y+F274Y+S224P, W292Y+F274Y+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+W266Y+R265K, W292Y+W266Y+S255P, W292Y+W266Y+T246N, W292Y+W266Y+T237N, W292Y+W266Y+S224P, W292Y+W266Y+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+R265K +S255P, W292Y+R265K+T246N, W292Y+R265K+T237N, W292Y+R265K+S224P, W292Y+R265K+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+S255P +T246N, W292Y+S255P+T237N, W292Y+S255P+S224P, W292Y+S255P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+T246N +T237N, W292Y+T246N+S224P, W292Y+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+T237N +S224P, W292Y+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+S224P +5221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+W266Y +R265K, F274Y+W266Y+S255P, F274Y+W266Y+T246N, F274Y+W266Y+T237N, F274Y+W266Y+S224P, F274Y+W266Y+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+R265K +S255P, F274Y+R265K+T246N, F274Y+R265K+T237N, F274Y+R265K+S224P, F274Y+R265K+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+S255P +T246N, F274Y+S255P+T237N, F274Y+S255P+S224P, F274Y+S255P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+T246N +T237N, F274Y+T246N+S224P, F274Y+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+T237N +S224P, F274Y+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+S224P +S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+R265K +S255P, W266Y+R265K+T246N, W266Y+R265K+T237N, W266Y+R265K+S224P, W266Y+R265K+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+S255P +T246N, W266Y+S255P+T237N, W266Y+S255P+S224P, W266Y+S255P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+T246N +T237N, W266Y+T246N+S224P, W266Y+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+T237N +S224P, W266Y+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+S224P +S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions R265K+S255P +T246N, R265K+S255P+T237N, R265K+S255P+S224P, R265K+S255P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions R265K+T246N +T237N, R265K+T246N+S224P, R265K+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions R265K+T237N +S224P, R265K+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions R265K+S224P +S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions S255P+T246N +T237N, S255P+T246N+S224P, S255P+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions S255P+T237N +S224P, S255P+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions S255P+S224P +S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions T246N+T237N +S224P, T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions T246N+S224P +S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions T237N+S224P +S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+F274Y +W266Y+R265K, W292Y+F274Y+W266Y+S255P, W292Y+F274Y+W266Y+T246N, W292Y+F274Y+W266Y+T237N, W292Y+F274Y+W266Y+S224P, W292Y+F274Y+W266Y+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+W266Y+R265K+S255P, W292Y+W266Y+R265K+T246N, W292Y+W266Y+R265K+T237N, W292Y+W266Y+R265K+S224P, W292Y+W266Y+R265K+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+R265K +S255P+T246N, W292Y+R265K+S255P+T237N, W292Y+R265K+S255P+S224P, W292Y+R265K+S255P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+S255P +T246N+T237N, W292Y+S255P+T246N+S224P, W292Y+S255P+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+T246N +T237N+S224P, W292Y+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+T237N +S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+W266Y +R265K+S255P, F274Y+W266Y+R265K+T246N, F274Y+W266Y+R265K+T237N, F274Y+W266Y+R265K+S224P, F274Y+W266Y+R265K+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+R265K +S255P+T246N, F274Y+R265K+S255P+T237N, F274Y+R265K+S255P+S224P, F274Y+R265K+S255P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+S255P +T246N+T237N, F274Y+S255P+T246N+S224P, F274Y+S255P+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+T246N +T237N+S224P, F274Y+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+T237N +S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+R265K +S255P+T246N, W266Y+R265K+S255P+T237N, W266Y+R265K+S255P+S224P, W266Y+R265K+S255P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+S255P +T246N+T237N, W266Y+S255P+T246N+S224P, W266Y+S255P+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+T246N +T237N+S224P, W266Y+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+T237N +S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions R265K+S255P +T246N+T237N, R265K+S255P+T246N+S224P, R265K+S255P+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions R265K+T246N +T237N+S224P, R265K+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions R265K+T237N +S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions S255P+T246N +T237N+S224P, S255P+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions S255P+T237N +S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions T246N+T237N +S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+F274Y +W266Y+R265K+S255P, W292Y+F274Y+W266Y+R265K+T246N, W292Y+F274Y+W266Y+R265K+T237N, W292Y+F274Y+W266Y+R265K+S224P, W292Y+F274Y+W266Y+R265K+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+W266Y+R265K+S255P+T246N, W292Y+W266Y+R265K+S255P+T237N, W292Y+W266Y+R265K+S255P+S224P, W292Y+W266Y+R265K+S255P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+R265K +S255P+T246N+T237N, W292Y+R265K+S255P+T246N+S224P, W292Y+R265K+S255P+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+S255P +T246N+T237N+S224P, W292Y+S255P+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+T246N +T237N+S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+W266Y +R265K+S255P+T246N, F274Y+W266Y+R265K+S255P+T237N, F274Y+W266Y+R265K+S255P+S224P, F274Y+W266Y+R265K+S255P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+R265K +S255P+T246N+T237N, F274Y+R265K+S255P+T246N+S224P, F274Y+R265K+S255P+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+S255P+T246N+T237N+S224P, F274Y+S255P+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+T246N +T237N+S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+R265K +S255P+T246N+T237N, W266Y+R265K+S255P+T246N+S224P, W266Y+R265K+S255P+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+S255P +T246N+T237N+S224P, W266Y+S255P+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+T246N +T237N+S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions R265K+S255P +T246N+T237N+S224P, R265K+S255P+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions R265K+T246N +T237N+S224P, R265K+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions S255P+T246N +T237N+S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+F274Y +W266Y+R265K+S255P+T246N, W292Y+F274Y+W266Y+R265K+S255P+T237N,

W292Y+F274Y+W266Y+R265K+S255P+S224P, W292Y+F274Y+W266Y+R265K+S255P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the W292Y+W266Y+R265K+S255P+T246N+T237N, W292Y+W266Y+R265K+S255P+T246N+S224P, W292Y+W266Y+R265K+S255P+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+R265K +S255P+T246N+T237N+S224P, W292Y+R265K+S255P+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+S255P +T246N+T237N+S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+W266Y +R265K+S255P+T246N+T237N, F274Y+W266Y+R265K+S255P+T246N+S224P, F274Y+W266Y+R265K+S255P+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+R265K +S255P+T246N+T237N+S224P, F274Y+R265K+S255P+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+S255P +T246N+T237N+S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+R265K+S255P+T246N+T237N+S224P, W266Y+R265K+S255P+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+S255P +T246N+T237N+S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions R265K+S255P +T246N+T237N+S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+F274Y +W266Y+R265K+S255P+T246N+T237N, W292Y+F274Y+W266Y+R265K+S255P+T246N+S224P, W292Y+F274Y+W266Y+R265K+S255P+T246N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the W292Y+W266Y+R265K+S255P+T246N+T237N+S224P, W292Y+W266Y+R265K+S255P+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+R265K +S255P+T246N+T237N+S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+W266Y +R265K+S255P+T246N+T237N+S224P, F274Y+W266Y+R265K+S255P+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+R265K +S255P+T246N+T237N+S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W266Y+R265K +S255P+T246N+T237N+S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+F274Y +W266Y+R265K+S255P+T246N+T237N+S224P, W292Y+F274Y+W266Y+R265K+S255P+T246N+T237N+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the W292Y+W266Y+R265K+S255P+T246N+T237N+S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions F274Y+W266Y +R265K+S255P+T246N+T237N+S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions W292Y+F274Y +W266Y+R265K+S255P+T246N+T237N+S224P+S221P of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 which has cellulase activity.

In another aspect, the variant comprises or consists of the substitutions selected from the following group:
W292Y
W266Y+W292Y
R265K+W292Y
R265K+W266Y+W292Y
F274Y+W292Y
W266Y+F274Y+W292Y
S221P+S224P+W292Y
S224P+T246N+W292Y
S255P+W292Y
W266Y+W292Y
   of the mature polypeptide of SEQ ID NO: 2.

The variants may further comprise one or more additional alterations at one or more (e.g., several) other positions.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In an aspect, the variant further comprises an alteration at position corresponding to positions 202.

Alternatively, the amino acid changes are of such a nature that the physico-chemical properties of the polypeptides are altered.

For example, the variants may comprise a substitution, insertion and/or deletion at one or more of the following positions: 4, 5, 6, 7, 8, 9, 10, 11, 12, 13, 14, 15, 16, 18, 19, 20, 21, 21a, 42, 44, 45, 47, 48, 49, 49a, 49b, 74, 82, 95j, 110, 111, 112, 113, 114, 115, 116, 119, 121, 123, 127, 128, 129, 130, 131, 132, 132a, 133, 145, 146, 147, 148, 149, 150b, 178, and/or 179 (cellulase numbering of WO 1998/012307, herein incorporated by reference).

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for cellulase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

The variants may consist of 200 to 315, e.g., 210 to 290, 215 to 295, 220 to 300 amino acids.

In an aspect, the variant has an improved property relative to the parent, wherein the improved property is improved biofinishing performance, a reduced weight loss of cellulose- containing textile, and/or a reduced loss in cellulose-containing textile strength.

In another aspect, biofinishing is biopolishing or denim abrasion.

### Parent Cellulases

In an aspect, the parent is GH45 cellulase.

The GH Family 45 cellulase enzymes (formerly Family K) act with inversion of anomeric configuration to generate the alpha-D anomer of the oligosaccharide as a product. It has been elucidated that, in the active site, one aspartic acid amino acid acts as a general acid and another as a general base.

The three dimensional structure of Family 45 enzymes has been elucidated (see, for example, the structure of Humicolainsolens in Davies et al, 1996, ActaCrystallographica Section D-Biological Crystallography 52:7-17 Part 1). The enzymes contain a six-stranded beta-barrel to which a seventh strand is appended. The structure contains both parallel and anti-parallel beta-strands. The active center is located in an open substrate-binding groove.

As used herein, the term “GH45 cellulase”, “Family 45 cellulase” or “Cel45” means a carbohydrate active cellulase enzyme that contains a glycoside hydrolase Family 45 catalytic domain that is classified under EC 3.2.1.4. The term encompasses a carbohydrate active enzyme that hydrolyzes cellulose and cello-oligosaccharides using an inverting mechanism, and has either of the following two signature sequences in the vicinity of the catalytic aspartic acid amino acids: (i) both a first conserved signature sequence of A/S/T-T-R/N/T-Y/F/T-X-D-X-X-X-X-X-C/A-A/G/S-W/C and a second conserved signature sequence of H/Q/D/N-F/L-D-I/L/F; or (ii) has the second conserved signature sequence of H/Q/D/N-F/L-D-I/L/F but lacks said first conserved sequence. In one embodiment, the second conserved signature sequence is H-F-D-I.

Family 45 cellulase enzymes have been divided into at least two subfamilies referred to as “A” and “B” (Igarashi et al, Applied and Environmental Microbiology, 2008, 74(18):5628-5634). According to one embodiment of the invention, the Family 45 cellulase enzyme is a subfamily A member. According to another embodiment of the invention, the Family 45 cellulase enzyme is a subfamily B member. This includes, but is not limited to, those subfamily A and subfamily B enzymes listed in the tables below.

### Family 45 Cellulase Subfamily B Members:

| | | GenBank Accession |
|---|---|---|
| Organism | Abreviated Name | Number |
| Trichoderma reesei | TrCel45A | CAA83846.1 |
| Trichomderma viride | TvEGV | AAQ21385.1 |
| Penicillium decumbens | PdCel 45A | ACF33814.1 |
| Aspergillus nidulans | AnAN6786.2 | EAA58604.1 |
| Hadiotis discus discus | HddEG1 | ABO26608.1 |
| Ampullaria crossean | AcEG27I | ABR92637.1 |
| Ampullaria crossean | AcEG27II | ABR92638.1 |
| Mytilus edulis | MeEG | CAC59695.1 |
| Phanerochaete chrysosporium | PcCel45A | BAG68300.1 |

Family 45 Cellulase Subfamily A Members:

| | | GenBank Accession Number and |
|---|---|---|
| Organism | Abreviated Name | WO publication Number |
| Humicola insolens | HiCel45A | AAE16508.1 |
| Humicola grisea var. thermoidea | HgEgl3 | BAA74956.1 |
| Humicola nigrescens | HnCel45A | CAB42308.1 |
| Geomyces pannorum | Gp RF6293 Cel45A | SEQ ID NO: 13 in WO 2010/076388 |
| Geomyces pannorum | Gp RF6293 Cel45B | SEQ ID NO: 15 in WO 2010/076388 |
| Fusarium cf. equiseti | Fe RF6318 Cel45B | SEQ ID NO: 17 in WO 2010/076388 |
| Geomyces pannorum | Gp RF6546 Cel45A | SEQ ID NO: 19 in WO 2010/076388 |
| Geomyces pannorum | Gp RF6608 Cel45A | SEQ ID NO: 21 in WO 2010/076388 |
| Geomyces pannorum | Gp RF6608 Cel45B | SEQ ID NO: 23 in WO 2010/076388 |
| Staphylotrichumcoccosporum | ScSTCE1 | BAG69187.1 |
| Staphylotrichum coccosporum | ScSTCE1 | SEQ ID NO: 3 in WO 2005/054475 |
| Sordaria fimicola | Sfcel45 | SEQ ID NO: 2 in WO 2014/026630 |
| Melanocarpus albomyces | MaCel45A | CAD56665.1 |
| Podospora anserina | PaCel45A | CAP61565.1 |
| Acremonium thermophilum | AtSEQ6 | ACE10216.1 |
| Thielavia terrestris | TtCel45A | SEQ ID NO: 4 in WO 2012/089024 |
| Trichothecium roseum | TroCel45A | CAB42312.1 |
| Acremonium thermophilum | AtSEQ2 | ABW41463.1 |
| Fusarium anguioides | FaCel45A | CAB42310.1 |
| Clonostachys rosea f. catenulata | CrCel45A | CAB42311.1 |
| Neurospora crassa | NcCEl45A | CAD70529.1 |
| Volutella colletotrichoides | VcSEQ22 | AAY00854.1 |
| Gibberella zeae | GzCel45A | AAR02399.1 |
| Fusarium oxysporum | FoCel45A | AAA65589.1 |
| Acremonium SP. | AsSEQ10 | AAY00848.1 |
| Acremonium SP. | AsSEQ8 | AAY00847.1 |
| Chrysosporium lucknowense | CICel45A | AAQ38150.1 |
| Thielavia heterothallica | ThSEQ2 | AAY00844.1 |
| Mucor circinelloides | McMce1 | BAD95808.1 |
| Reticulitermes speratus | RshpCel45A | BAA98037.1 |
| Bursaphelenchus xylophilus | BxEng1 | BAD34546.1 |
| Botryotinia fuckeliana | BfCel45A | XP_JX11547700.1 |
| Acremonium thermophilum | AtSEQ4 | ABW41464.1 |
| Scopulariopsis brevicaulis | SbEgl | Q7M4T4* |
| Syncephalastrum racemosum | SrCBHI | ABU49185.2 |
| Rhizopus oryzae | RoRce1 | BAC53956.1 |
| Crinipellis scabella | CsSEQ16 | AAY00851.1 |
| Macrophomina phaseolina | MpSEQ14 | AAY00850.1 |
| Podospora anserina | PaCel45B | CAP69443.1 |
| Rhizopus oryzae | RoRce3 | BAC53988.1 |
| Bursaphelenchus xylophilus | BxEng2 | BAD34544.1 |
| Bursaphelenchus xylophilus | BxEng3 | BAD34548.1 |
| Humicolagrisea var. thermoidea | HgEgl4 | BAA74957.1 |
| Phycomyces nitens | PnPcel | BAD77808.1 |
| Rhizopus oryzae | RoRce2 | BAC53987.1 |
| Mastotermes darwiniensis | MdhsFm4 | CAD39200.1 |
| hindgut symbiont sp. | | |
| Magnaporthe grisea | MgCel45A | XP_363402.1 |
| Mastotermes darwiniensis | MdhsFm3 | CAD39199.1 |
| hindgut symbiont sp. | | |
| Mastotermes darwiniensis | MdhsFml | CAD39197.1 |
| hindgut symbiont sp. | | |
| Mastotermes darwiniensis | MdhsFm2 | CAD39198.1 |
| hindgut symbiont sp. | | |
| Neurospora tetrasperma | Ntcel45 | SEQ ID NO: 2 in WO 2015/058700 |
| Pichia pastoris GS115 | PpCel45A | CAY71902.1 |
| Piromyces equi | PeCel45A | CAB92325.1 |
| Apriona germari | AgCelII | AAN78326.1 |
| Apriona germari | AgCelII | AAR22385.1 |
| Alternaria alternata | AaKl | AAF05700.1 |
| Phaedon cochleariae | PcEg | CAA76931.1 |
| Talaromyces emersonii | TeCel45A | CAJ75963.1 |
| Ustilago maydis | UmEglI | AAB36147.1 |

*Uniprot entry

In an aspect, the parent cellulases comprises a catalytic domain and a cellulase binding domain, wherein the cellulase binding domain is heterologous or homologous to the catalytic domain.

In a further aspect, a linker is between the catalytic domain and the cellulose binding domain.

In a further aspect, the linker is heterologous or homologous to the cellulose binding domain.

In a further aspect, the linker is heterologous or homologous to the catalytic domain.

In another embodiment, the parent cellulase is selected from the group consisting of:
a. a polypeptide having at least 60% sequence identity to the mature polypeptide of SEQ ID NO: 2, SEQ ID NO: 4, SEQ ID NO: 8, SEQ ID NO: 12, SEQ ID NO: 13, SEQ ID NO: 14; or SEQ ID NO: 18;
a polypeptide encoded by a polynucleotide that hybridizes under low stringency conditions with (i) the mature polypeptide coding sequence of SEQ ID NO: 1, SEQ ID NO: 3, SEQ ID NO: 7, SEQ ID NO: 37, SEQ ID NO: 38, SEQ ID NO: 39 or SEQ ID NO: 17, (ii) the cDNA sequence thereof, or (iii) the full-length complement of (i) or (ii);
a polypeptide encoded by a polynucleotide having at least 60% identity to the mature polypeptide coding sequence of SEQ ID NO: 1, SEQ ID NO: 3, SEQ ID NO: 7, SEQ ID NO: 37, SEQ ID NO: 38, SEQ ID NO: 39 or SEQ ID NO: 17, or the cDNA sequence thereof; and
a fragment of the mature polypeptide of SEQ ID NO: 2, SEQ ID NO: 4, SEQ ID NO: 8, SEQ ID NO: 12, SEQ ID NO: 13, SEQ ID NO: 14; or SEQ ID NO: 18, which has cellulase activity.

In a further aspect, the parent cellulase has at least 60%, e.g., at least 65%, at least 70%, at least 75%, at least 80%, at least 85%, at least 90%, at least 95%, at least 96%, at least 97%, at least 98%, at least 99% or 100% sequence identity to the mature polypeptide of SEQ ID NO: 2, SEQ ID NO: 4, SEQ ID NO: 8, SEQ ID NO: 12, SEQ ID NO: 13, SEQ ID NO: 14; or SEQ ID NO: 18.

In a further embodiment, the parent cellulase is encoded by a polynucleotide that hybridizes under low stringency conditions, medium stringency conditions, medium-high stringency conditions, high stringency conditions, or very high stringency conditions with (i) the mature polypeptide coding sequence of SEQ ID NO: 1, SEQ ID NO: 3, SEQ ID NO: 7, SEQ ID NO: 37, SEQ ID NO: 38, SEQ ID NO: 39 or SEQ ID NO: 17, or (ii) the full-length complement of (i).

In a further aspect, the parent cellulase is encoded by a polynucleotide having at least 60%, e.g., at least 65%, at least 70%, at least 75%, at least 80%, at least 85%, at least 90%, at least 95%, at least 96%, at least 97%, at least 98%, at least 99%, or 100% sequence identity to the mature polypeptide coding sequence of SEQ ID NO: 1, SEQ ID NO: 3, SEQ ID NO: 7, SEQ ID NO: 37, SEQ ID NO: 38, SEQ ID NO: 39 or SEQ ID NO: 17, or the cDNA sequence thereof.

In a further aspect, the parent cellulase comprises or consists of the mature polypeptide of SEQ ID NO: 2, SEQ ID NO: 4, SEQ ID NO: 8, SEQ ID NO: 12, SEQ ID NO: 13, SEQ ID NO: 14; or SEQ ID NO: 18.

In a further aspect, the parent cellulase is a fragment of the mature polypeptide of SEQ ID NO: 2, SEQ ID NO: 4, SEQ ID NO: 8, SEQ ID NO: 12, SEQ ID NO: 13, SEQ ID NO: 14; or SEQ ID NO: 18, wherein the fragment has cellulase activity.

In another aspect, the parent is a fragment of the mature polypeptide of SEQ ID NO: 2 containing 200 to 315, e.g., 210 to 290, 215 to 295, 220 to 300 amino acid residues.

In another embodiment, the parent is an allelic variant of the mature polypeptide of SEQ ID NO: 2, SEQ ID NO: 4, SEQ ID NO: 8, SEQ ID NO: 12, SEQ ID NO: 13, SEQ ID NO: 14; or SEQ ID NO: 18.

The polynucleotide of SEQ ID NO: 1, SEQ ID NO: 3, SEQ ID NO: 7, SEQ ID NO: 37, SEQ ID NO: 38, SEQ ID NO: 39 or SEQ ID NO: 17 or a subsequence thereof, as well as the polypeptide of SEQ ID NO: 4, SEQ ID NO: 8, SEQ ID NO: 12, SEQ ID NO: 13, SEQ ID NO: 14; or SEQ ID NO: 18 or a fragment thereof, may be used to design nucleic acid probes to identify and clone DNA encoding a parent from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a parent. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1, SEQ ID NO: 3, SEQ ID NO: 7, SEQ ID NO: 37, SEQ ID NO: 38, SEQ ID NO: 39 or SEQ ID NO: 17 or a subsequence thereof, the carrier material is used in a Southern blot.

For purposes of the present invention, hybridization indicates that the polynucleotide hybridizes to a labeled nucleic acid probe corresponding to (i) SEQ ID NO: 1, SEQ ID NO: 3, SEQ ID NO: 7, SEQ ID NO: 37, SEQ ID NO: 38, SEQ ID NO: 39 or SEQ ID NO: 17; (ii) the mature polypeptide coding sequence of SEQ ID NO: 1, SEQ ID NO: 3, SEQ ID NO: 7, SEQ ID NO: 37, SEQ ID NO: 38, SEQ ID NO: 39 or SEQ ID NO: 17; (iii) the cDNA sequence thereof; (iv) the full-length complement thereof; or (v) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is the mature polypeptide coding sequence of SEQ ID NO: 1, SEQ ID NO: 3, SEQ ID NO: 7, SEQ ID NO: 37, SEQ ID NO: 38, SEQ ID NO: 39 or SEQ ID NO: 17. In another aspect, the nucleic acid probe is nucleotides 64 to 1101 of SEQ ID NO: 1. In another aspect, the nucleic acid probe is a polynucleotide that encodes the polypeptide of SEQ ID NO: 2; the mature polypeptide thereof; or a fragment thereof. In another aspect, the nucleic acid probe is SEQ ID NO: 1, SEQ ID NO: 3, SEQ ID NO: 7, SEQ ID NO: 37, SEQ ID NO: 38, SEQ ID NO: 39 or SEQ ID NO: 17 or the cDNA sequence thereof.

The parent may be a hybrid polypeptide in which a region of one polypeptide is fused at the N-terminus or the C-terminus of a region of another polypeptide.

The parent may be a fusion polypeptide or cleavable fusion polypeptide in which a region another polypeptide is fused at the N-terminus or the C-terminus of a polypeptide. A fusion polypeptide is produced by fusing a polynucleotide encoding another polypeptide to a polynucleotide of the present invention. in which a region of one polypeptide (for example, cellulase binding domain of a cellulase) is added or fused to the N-terminus or the C-terminus of a region of another polypeptide, for example, a catalytic domain and linker of another cellulase)Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent may be obtained from microorganisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly.

In another aspect, the parent is Thielavia cellulase, Staphylotrichum cellulase, Neurospora cellulase, or Thielavia cellulose. Preferably, the parent is a Staphylotrichum coccosporum cellulase, Neurospora tetrasperma cellulase, Thielavia terrestris or Thielavia hyrcaniae cellulase.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding a parent may then be obtained by similarly screening a genomic DNA or cDNA library of another microorganism or mixed DNA sample. Once a polynucleotide encoding a parent has been detected with the probe(s), the polynucleotide can be isolated or cloned by utilizing techniques that are known to those of ordinary skill in the art (see, e.g., Sambrook et al., 1989, supra).

The present invention further relates to a variant of a parent GH45 cellulase, comprising a catalytic domain, and a cellulose binding domain, wherein the cellulase binding domain is heterologous to the catalytic domain, and wherein the variant has an improved biofinishing activity compared with the parent GH45 cellulase.

In one aspect, a linker is between the catalytic domain and the cellulose binding domain.

In one embodiment, the linker is homologous to the cellulose binding domain.

In one embodiment, the linker is homologous to the catalytic domain.

In one embodiment, the catalytic domain is selected from the group consisting of:
a. a polypeptide having at least 60% sequence identity to the catalytic domain of SEQ ID NO: 12, SEQ ID NO: 13, or SEQ ID NO: 14;
b. a polypeptide encoded by a polynucleotide that hybridizes under low stringency conditions with (i) the catalytic domain coding sequence of SEQ ID NO: 37, SEQ ID NO: 38, or SEQ ID NO: 39, (ii) the cDNA sequence thereof, or (iii) the full-length complement of (i) or (ii);
c. a polypeptide encoded by a polynucleotide having at least 60% identity to the catalytic domain coding sequence of SEQ ID NO: 37, SEQ ID NO: 38, SEQ ID NO: 39, or the cDNA sequence thereof; and
d. a fragment of the catalytic domain of SEQ ID NO: 12, SEQ ID NO: 13, or SEQ ID NO: 14, which has cellulase activity.

In one embodiment, the catalytic domain has at least 60%, e.g., at least 65%, at least 70%, at least 75%, at least 80%, at least 85%, at least 90%, at least 95%, at least 96%, at least 97%, at least 98%, at least 99% or 100% sequence identity to the catalytic domain of SEQ ID NO: 12, SEQ ID NO: 13, or SEQ ID NO: 14.

In one embodiment, catalytic domain is encoded by a polynucleotide that hybridizes under low stringency conditions, medium stringency conditions, medium-high stringency conditions, high stringency conditions, or very high stringency conditions with (i) the catalytic domain coding sequence of SEQ ID NO: 37, SEQ ID NO: 38, SEQ ID NO: 39 or (ii) the full- length complement of (i).

In one embodiment, the catalytic domain is encoded by a polynucleotide having at least 60%, e.g., at least 65%, at least 70%, at least 75%, at least 80%, at least 85%, at least 90%, at least 95%, at least 96%, at least 97%, at least 98%, at least 99%, or 100% sequence identity to the catalytic domain coding sequence of SEQ ID NO: 37, SEQ ID NO: 38, SEQ ID NO: 39 or the cDNA sequence thereof.

In one embodiment, the catalytic domain comprises or consists of amino acids 22-223 of SEQ ID NO: 12, amino acids 1-200 of SEQ ID NO: 13, or amino acids 22-223 of SEQ ID NO: 14.

In one embodiment, the cellulose binding domain is selected from the group consisting of:
a. a polypeptide having at least 60% sequence identity to the cellulose binding domain of SEQ ID NO: 12, SEQ ID NO: 13, SEQ ID NO: 14 or SEQ ID NO: 8;
b. a polypeptide encoded by a polynucleotide that hybridizes under low stringency conditions with (i) the cellulose binding domain coding sequence of SEQ ID NO: 37, SEQ ID NO: 38, SEQ ID NO: 39 or SEQ ID NO: 7, (ii) the cDNA sequence thereof, or (iii) the full-length complement of (i) or (ii);
c. a polypeptide encoded by a polynucleotide having at least 60% identity to the cellulose binding domain coding sequence of SEQ ID NO: 37, SEQ ID NO: 38, SEQ ID NO: 39 or SEQ ID NO: 7, or the cDNA sequence thereof; and
d. a fragment of the cellulose binding domain of SEQ ID NO: 12, SEQ ID NO: 13, SEQ ID NO: 14 or SEQ ID NO: 8, which has the cellulose binding activity.

In one embodiment, the cellulose binding domain has at least 60%, e.g., at least 65%, at least 70%, at least 75%, at least 80%, at least 85%, at least 90%, at least 95%, at least 96%, at least 97%, at least 98%, at least 99% or 100% sequence identity to the cellulose binding domain of SEQ ID NO: 12, SEQ ID NO: 13, SEQ ID NO: 14 or SEQ ID NO: 8.

In one embodiment, the cellulose binding domain is encoded by a polynucleotide that hybridizes under low stringency conditions, medium stringency conditions, medium-high stringency conditions, high stringency conditions, or very high stringency conditions with (i) the cellulose binding domain coding sequence of SEQ ID NO: 37, SEQ ID NO: 38, SEQ ID NO: 39, SEQ ID NO: 7 or (ii) the full-length complement of (i).

In one embodiment, the cellulose binding domain is encoded by a polynucleotide having at least 60%, e.g., at least 65%, at least 70%, at least 75%, at least 80%, at least 85%, at least 90%, at least 95%, at least 96%, at least 97%, at least 98%, at least 99%, or 100% sequence identity to the cellulose binding domain coding sequence of SEQ ID NO: 37, SEQ ID NO: 38, SEQ ID NO: 39, or SEQ ID NO: 7 or the cDNA sequence thereof.

In one embodiment, the cellulose binding domain comprises or consists of amino acids 262-299 of SEQ ID NO: 12, amino acids 258-295 of SEQ ID NO: 13, amino acids 257-293 of SEQ ID NO: 14, or amino acids 249-286 of SEQ ID NO: 8.

In one embodiment, linker is selected from the group consisting of:
a. a polypeptide having at least 60% sequence identity to the linker of SEQ ID NO: 12, SEQ ID NO: 13, SEQ ID NO: 14 or SEQ ID NO: 8;
b. a polypeptide encoded by a polynucleotide that hybridizes under low stringency conditions with (i) the linker coding sequence of SEQ ID NO: 37, SEQ ID NO: 38, SEQ ID NO: 39 or SEQ ID NO: 7, (ii) the cDNA sequence thereof, or (iii) the full-length complement of (i) or (ii);
c. a polypeptide encoded by a polynucleotide having at least 60% identity to the linker coding sequence of SEQ ID NO: 37, SEQ ID NO: 38, SEQ ID NO: 39 or SEQ ID NO: 7, or the cDNA sequence thereof; and
d. a fragment of the linker of SEQ ID NO: 12, SEQ ID NO: 13, SEQ ID NO: 14 or SEQ ID NO: 8, which has the cellulose binding activity.

In one embodiment, the linker has at least 60%, e.g., at least 65%, at least 70%, at least 75%, at least 80%, at least 85%, at least 90%, at least 95%, at least 96%, at least 97%, at least 98%, at least 99% or 100% sequence identity to the linker of SEQ ID NO: 12, SEQ ID NO: 13, SEQ ID NO: 14 or SEQ ID NO: 8.

In one embodiment, the linker is encoded by a polynucleotide that hybridizes under low stringency conditions, medium stringency conditions, medium-high stringency conditions, high stringency conditions, or very high stringency conditions with (i) the linker coding sequence of SEQ ID NO: 37, SEQ ID NO: 38, SEQ ID NO: 39, SEQ ID NO: 7 or (ii) the full-length complement of (i).

In one embodiment, the linker is encoded by a polynucleotide having at least 60%, e.g., at least 65%, at least 70%, at least 75%, at least 80%, at least 85%, at least 90%, at least 95%, at least 96%, at least 97%, at least 98%, at least 99%, or 100% sequence identity to the linker coding sequence of SEQ ID NO: 37, SEQ ID NO: 38, SEQ ID NO: 39, or SEQ ID NO: 7 or the cDNA sequence thereof.

In one embodiment, the linker comprises or consists of amino acids 224-261 of SEQ ID NO: 12, amino acids 201-257 of SEQ ID NO: 13, amino acids 224-256 of SEQ ID NO: 14, or amino acids 203-248 of SEQ ID NO: 8.

In one embodiment, the variant has at least 60%, e.g., at least 65%, at least 70%, at least 75%, at least 80%, at least 85%, at least 90%, at least 95% identity, at least 96%, at least 97%, at least 98%, or at least 99%, but less than 100%, sequence identity to the amino acid sequence of the parent cellulase.

In one embodiment, the variant consists of 200 to 315, e.g., 210 to 290, 215 to 295, 220 to 300 amino acids.

In one embodiment, the variant comprises an alteration at one or more positions corresponding to positions 292, 274, 266, 265, 255, 246, 237, 224 and 221 of the mature polypeptide of SEQ ID NO: 2.

In one embodiment, the number of alterations is 1-20, e.g., 1-10 and 1-5, such as 1, 2, 3, 4, 5, 6, 7, 8, 9 or 10 alterations.

### Preparation of Variants

The present invention also relates to methods for obtaining a variant having cellulase activity, comprising: (a) introducing into a parent cellulase an alteration at one or more (e.g., several) positions corresponding to positions 292, 274, 266, 265, 255, 246, 237, 224 and 221 of the mature polypeptide of SEQ ID NO: 2, wherein the variant has cellulase activity; and (b) recovering the variant.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

The present invention further relates to a method for obtaining a variant, comprising replacing a cellulose binding domain a parent cellulase with a heterologous cellulose binding domain; or adding a heterologous cellulose binding domain to a parent cellulase which does not comprises a cellulose binding domain. Techniques for producing the variants are known in the art, and include ligating the coding sequences so that they are in frame and that expression of the variant is under control of the same promoter(s) and terminator. Variant may also be constructed using intein technology in which variants are created post-translationally (Cooper et al., 1993, EMBO J. 12: 2575-2583; Dawson et al., 1994, Science 266: 776-779).

### Polynucleotides

The present invention also relates to polynucleotides encoding a variant of the present invention.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide which is recognized by a host cell for expression of the polynucleotide. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryllIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator sequence is operably linked to the 3′-terminus of the polynucleotide encoding the variant. Any terminator that is functional in the host cell may be used.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryllIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader sequence is operably linked to the 5′-terminus of the polynucleotide encoding the variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the variant-encoding sequence and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of the variant and the signal peptide sequence is positioned next to the N-terminus of the propeptide sequence.

It may also be desirable to add regulatory sequences that regulate expression of the variant relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked with the regulatory sequence.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide encoding a variant of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausfi, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804) or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series Nov. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a variant, comprising: (a) cultivating a host cell of the present invention under conditions suitable for expression of the variant; and (b) recovering the variant.

The host cells are cultivated in a nutrient medium suitable for production of the variant using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the variants. These detection methods include, but are not limited to, use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, collection, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

### Textile

As used herein, the term “textile” refers to fibers, yarns, fabrics, garments, and non- wovens. The term encompasses textiles made from natural, synthetic (e.g., manufactured), and various natural and synthetic blends. Textiles may be unprocessed or processed fibers, yarns, woven or knit fabrics, non-wovens, and garments and may be made using a variety of materials, some of which are mentioned, herein.

The process of the invention is most beneficially applied to cellulose-containing textile, such as cotton, viscose, rayon, ramie, linen, Tencel, or mixtures thereof, or mixtures of any of these fibres, or mixtures of any of these fibres together with synthetic fibres such as mixtures of cotton and spandex (stretch-denim). In particular, the fabric is dyed fabric. In an embodiment, the fabric is denim. The denim fabric may be dyed with vat dyes such as indigo, or indigo-related dyes such as thioindigo.

In an embodiment of the process of the invention, cellulose-containing textile is cotton- containing textile or man-made cellulose-containing textile.

### Measurement of the Biofinishing Activity of Cellulase Variants

In order to determine the biofinishing activity of the cellulase variants, they are typically purified using known techniques.

The term “biofinishing” as used herein refers to the treatment of textile using cellulases and includes, but not limited to, biopolishing and biostoning.

The “biofinishing activity”, especially “biopolishing activity”, as used herein, is determined as set forth in Examples. The biopolishing effectiveness of the GH45 cellulases can be measured by the activity in removing fuzz, or small balls of fuzz (referred to as pills), from fabric. The depilling can be expressed as the depilling activity per unit of protein (i.e., specific depilling activity).

According to one embodiment of the invention, an assay that measures biofinishing activity is a pilling note test for biopolishing activity. GH45 cellulase variants of the present invention provide for enhanced biofinishing of cotton-containing textile relative to the biofinishing effect of parent GH45 cellulase. For example, GH45 cellulase variants delivers about 1%, about 5%, about 10%, about 15%, about 20%, about 30%, about 50% stronger biopolishing effect than the parent GH45 cellulases. GH45 cellulase variants delivers about 1%, about 5%, about 10%, about 15%, about 20%, about 30%, about 50% stronger biostoning effect than the parent GH45 cellulases in denim abrasion. In a pilling notes test, GH45 cellulase variants delivers about 0.1, about 0.2, about 0.5, about 0.8, about 1.0 pilling note more than that of the parent GH45 cellulases. The utilization of such GH45 cellulase variants could be a step forward with respect to improving process economics.

### Textile Manufacturing Process

The processing of a fabric, such as of a cellulosic material, into material ready for garment manufacture involves several steps: spinning of the fiber into a yarn; construction of woven or knit fabric from the yarn; and subsequent preparation processes, dyeing/printing and finishing operations. Preparation processes are necessary for removing natural and man-induced impurities from fibers and for improving their aesthetic appearance and processability prior to for instance dyeing/printing and finishing. Common preparation processes comprise desizing (for woven goods), scouring, and bleaching, which produce a fabric suitable for dyeing or finishing.

Woven fabric is constructed by weaving “filling” or “weft” yarns between warp yarns stretched in the longitudinal direction on the loom. The warp yarns must be sized before weaving in order to lubricate and protect them from abrasion at the high speed insertion of the filling yarns during weaving. Common size agents are starches (or starch derivatives and modified starches), poly(vinyl alcohol), carboxyl methyl cellulose (i.e., CMC) where starches are dominant. Paraffin, acrylic binders and variety of lubricants are often included in the size mix. The filling yarn can be woven through the warp yarns in a “over one—under the next” fashion (plain weave) or by “over one—under two” (twill) or any other myriad of permutations. Generally, dresses, shirts, pants, sheeting's, towels, draperies, etc. are produced from woven fabric. After the fabric is made, size on the fabric must be removed again (i.e., desizing).

Knitting is forming a fabric by joining together interlocking loops of yarn. As opposed to weaving, which is constructed from two types of yarn and has many “ends”, knitted fabric is produced from a single continuous strand of yarn. As with weaving, there are many different ways to loop yarn together and the final fabric properties are dependent both upon the yarn and the type of knit. Underwear, sweaters, socks, sport shirts, sweat shirts, etc. are derived from knit fabrics.

### Desizing

Desizing is the degradation and/or removal of sizing compounds from warp yarns in a woven fabric. Starch is usually removed by an enzymatic desizing procedure. In addition, oxidative desizing and chemical desizing with acids or bases are sometimes used.

In some embodiments, the desizing enzyme is an amylolytic enzyme, such as an alpha- amylase, a beta-amylase, a mannanase, a glucoamylase, or a combination thereof.

Suitable alpha and beta-amylases include those of bacterial or fungal origin, as well as chemically or genetically modified mutants and variants of such amylases. Suitable alpha- amylases include alpha-amylases obtainable from Bacillus species. Suitable commercial amylases include but are not limited to OPTISIZE® NEXT, OPTISIZE® FLEX and OPTISIZE® COOL (all from Genencor International Inc.), and DURAMYL™, ERMAMYL™, FUNGAMYL™ TERMAMYL™, AQUAZYME™ and BAN™ (all available from Novozymes A/S, Bagsvaerd, Denmark).

Other suitable amylolytic enzymes include the CGTases (cyclodextrin glucanotransferases, EC 2.4.1.19), e.g., those obtained from species of Bacillus, Thermoanaerobactor or Thermoanaero-bacterium.

### Scouring

Scouring is used to remove impurities from the fibers, to swell the fibers and to remove seed coat. It is one of the most critical steps. The main purposes of scouring is to a) uniformly clean the fabric, b) soften the motes and other trashes, c) improve fabric absorbency, d) saponify and solubilize fats, oils, and waxes, and e) minimize immature cotton. Sodium hydroxide scouring at about boiling temperature is the accepted treatment for 100% cotton, while calcium hydroxide and sodium carbonate are less frequently used. Synthetic fibers are scoured at much milder conditions. Surfactant and chelating agents are essential for alkaline scouring. Enzymatic scouring has been introduced, wherein cellulase, hemicellulase, pectinase, lipase, and protease are all reported to have scouring effects.

### Bleaching

Bleaching is the destruction of pigmented color and/or colored impurities as well as seed coat fragment removal. Bleaching is performed by the use of oxidizing or reducing chemistry. Oxidizing agents can be further subdivided into those that employ or generate: a) hypochlorite (OCl^{—}), b) chloride dioxide (ClO₂), c) permanganate (MnO₄—), d) ozone, and hydroperoxide species (OOH^{—} and/or OOH). Reducing agents are typical sulfur dioxide, hydrosulfite salts, etc. Enzymatic bleaching using glucose oxidase or peroxidase (for example, see WO 2013/040991) has been reported. Traditionally, hydrogen peroxide is used in this process.

### Printing and Dyeing

Printing and dyeing of textiles is carried out by applying dyes to the textile by any appropriate method for binding the dyestuff to the fibres in the textiles. The dyeing of textiles may for example be carried out by passing the fabric through a concentrated solution of dye, followed by storage of the wet fabric in a vapour tight enclosure to permit time for diffusion and reaction of the dye with the fabric substrate prior to rinsing off un-reacted dye. Alternatively, the dye may be fixed by subsequent steaming of the textile prior to rinsing. The dyes include synthetic and natural dyes. Typical dyes are those with anionic functional groups (e.g., acid dyes, direct dyes, Mordant dyes and reactive dyes), those with cationic groups (e.g., basic dyes), those requiring chemical reaction before application (e.g., vat dyes, sulphur dyes and azoic dyes), disperse dyes and solvent dyes.

Excess soluble dyestuff not bound to the fibres must be removed after dyeing to ensure fastness of the dyed textiles and to prevent unwanted dye transfer during laundering of the textiles by the consumer. Generally, a large amount of water is required for complete removal of excess dye. In a conventional process, the printed or dyed textile is first rinsed with cold water, then washed at high temperature with the addition of a suitable additive to decrease back- staining, like poly(vinylpyrrolidone) (PVP).

An enzymatic process for removal of excess dye from dyed fabric with a rinse liquor comprising at least one peroxidase, an oxidase agent and at least one mediator, such as liquor comprising a peroxidase, hydrogen peroxidise and a mediator like 1-hydroxy-benzotriazole is disclosed in WO 99/34054.

### Biopolishing

Most cotton fabrics and cotton blend fabrics have a hand-feeling problem that is rather hard and stiff without the application of finishing components. The fabric surface also is not smooth because small fuzzy microfibrils protrude from it. In addition, after a relatively short period of wear, pilling appears on the fabric surface thereby giving it an unappealing, worn look.

Biopolishing is a method to treat cellulosic fabrics during their manufacture by enzymes such as cellulases, which improves fabric quality with respect to “reduced pilling formation”. The most important effects of biopolishing can be characterized by less fuzz and pilling, increased gloss/luster, improved fabric handle, increased durable softness and/or improved water absorbency. Biopolishing usually takes place in the wet processing of the manufacture of knitted and woven fabrics or garments. Wet processing comprises such steps as e.g., desizing, scouring, bleaching, washing, dying/printing and finishing. Biopolishing could be performed as a separate step after any of the wetting steps or in combination with any of those wetting steps.

In the present invention, the step of biofinishing is carried out before, during or after step of desizing, bleaching, or printing and dyeing.

### Manufacturing of Denim Fabric

Some dyed fabric such as denim fabric, requires that the yarns are dyed before weaving. For denim fabric, the warp yarns are dyed for example with indigo, and sized, before weaving. Preferably the dyeing of the denim yarn is a ring-dyeing. A preferred embodiment of the invention is ring-dyeing of the yarn with a vat dye such as indigo, or an indigo-related dye such as thioindigo, or a sulfur dye, or a direct dye, or a reactive dye, or a naphthol. The yarn may also be dyed with more than one dye, e.g., first with a sulphur dye and then with a vat dye, or vice versa.

Preferably, the yarns undergo scouring and/or bleaching before they are dyed, in order to achieve higher quality of denim fabric. In general, after woven into dyed fabric, such as denim, the dyed fabric or garment proceeds to a desizing stage, preferably followed by a stoning or abrasion step and/or a color modification step.

The desizing process as used herein is the same process as mentioned above in the text.

After desizing, the dyed fabric undergoes a biostoning step. The biostoning step can be performed with enzymes or pumice stones or both. As used herein, the term “biostoning”, “stone washing” and “abrasion” are interchangeable, which means agitating the denim in an aqueous medium containing a mechanical abrasion agent such as pumice, an abrading cellulase or a combination of these, to provide a “stone-washed” look. In all cases, mechanical action is needed to remove the dye, and the treatment is usually carried out in washing machines, like drum washers, belly washers. As a result of uneven dye removal there are contrasts between dyed areas and areas from which dye has been removed. Treatment with cellulase can completely replace treatment with pumice stones. However, cellulase treatment can also be combined with pumice stone treatment, when it is desired to produce a heavily abraded finish. For denim manufacture, “biofinishing” includes “biostoning”.

Preferably, the abrasion is followed by a color modification step. As used herein, the terms “color modification” or “color adjustment” are used without distinction to refer to any change to the color of a textile resulting from the destruction, modification, or removal of a dyestuff associated with the textile. Without being limited to a theory, it is proposed that color modification results from the modification of chromaphores associated with a textile material, thereby changing its visual appearance. The chromophores may be naturally-associated with the material used to manufacture a textile (e.g., the white color of cotton) or associated with special finishes, such as dying or printing. Color modification encompasses chemical modification to a chromophore as well as chemical modification to the material to which a chromophore is attached.

Getting faded or bleached look in certain areas on textile especially denim, is an important part in textile manufacturing. This is normally achieved by applying KMnO₄ (or KMnO₄/H₃PO₄) solution (via brushing, rubbing or spray) onto dried denim after abrasion step. The stained area would get bleached after drying and washing with Na₂S₂O₅ solution. During this process indigo/sulphur dyes are destroyed by KMnO₄ through oxidation, and then Na₂S₂O₅ washing is applied to get rid of the brown colour caused by products of the oxidation. Such treatment will form a local color modification, i.e., a specific bleached pattern on denim to meet the customers' needs. In the present invention, the step of biofinishing is carried out before, during or after step of desizing, or color modification.

The present invention also relates to a process for biofinishing, comprising treating a cellulose-containing textile with variant of present invention.

The present invention further relates to a process for treating cellulose-containing textile, comprising
(a) desizing;
(b) color modification;
wherein the variant of the present invention is added before, during or after step (a) and step (b).

The present invention further relates to a process for treating cellulose-containing textile, comprising
(a) desizing;
(b) scouring;
(c) bleaching;
(d) dyeing;
wherein the variant of the present invention is added before, during or after step (a), (b), (c) or (d).

In an embodiment, the cellulase is treated or added under the low mechanical actions. Low mechanical actions can be carried out by applying low agitation speed or low mechanical substance, for example, rubber balls, to a system comprising cellulase and cellulose-containing textile.

The present invention relates to use of the variant of the present invention for biofinishing of cellulose-containing textile.

### Plants

The present invention also relates to plants, e.g., a transgenic plant, plant part, or plant cell, comprising a polynucleotide of the present invention so as to express and produce the variant in recoverable quantities. The variant may be recovered from the plant or plant part. Alternatively, the plant or plant part containing the variant may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats.

Also included within the scope of the present invention are the progeny of such plants, plant parts, and plant cells.

The transgenic plant or plant cell expressing a variant may be constructed in accordance with methods known in the art. In short, the plant or plant cell is constructed by incorporating one or more expression constructs encoding a variant into the plant host genome or chloroplast genome and propagating the resulting modified plant or plant cell into a transgenic plant or plant cell.

The expression construct is conveniently a nucleic acid construct that comprises a polynucleotide encoding a variant operably linked with appropriate regulatory sequences required for expression of the polynucleotide in the plant or plant part of choice. Furthermore, the expression construct may comprise a selectable marker useful for identifying plant cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences, is determined, for example, on the basis of when, where, and how the variant is desired to be expressed. For instance, the expression of the gene encoding a variant may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, or the rice actin 1 promoter may be used (Franck et al., 1980, Cell 21: 285-294; Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant Cell Physiol. 39: 885-889), a Vicia faba promoter from the legumin B4 and the unknown seed protein gene from Vicia faba (Conrad et al., 1998, J. Plant Physiol. 152: 708-711), a promoter from a seed oil body protein (Chen et al., 1998, Plant Cell Physiol. 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiol. 102: 991-1000), the chlorella virus adenine methyltransferase gene promoter (Mitra and Higgins, 1994, Plant Mol. Biol. 26: 85-93), the aldP gene promoter from rice (Kagaya et al., 1995, Mol. Gen. Genet. 248: 668-674), or a wound inducible promoter such as the potato pin2 promoter (Xu et al., 1993, Plant Mol. Biol. 22: 573-588). Likewise, the promoter may be induced by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a variant in the plant. For instance, the promoter enhancer element may be an intron that is placed between the promoter and the polynucleotide encoding a variant. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Agrobacterium tumefaciens-mediated gene transfer is a method for generating transgenic dicots (for a review, see Hooykas and Schilperoort, 1992, Plant Mol. Biol. 19: 15-38) and for transforming monocots, although other transformation methods may be used for these plants. A method for generating transgenic monocots is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al., 1992, Bio/Technology 10: 667-674). An alternative method for transformation of monocots is based on protoplast transformation as described by Omirulleh et al., 1993, Plant Mol. Biol. 21: 415-428. Additional transformation methods include those described in U.S. Pat. Nos. 6,395,966 and 7,151,204 (both of which are herein incorporated by reference in their entirety).

Following transformation, the transformants having incorporated the expression construct are selected and regenerated into whole plants according to methods well known in the art. Often the transformation procedure is designed for the selective elimination of selection genes either during regeneration or in the following generations by using, for example, co- transformation with two separate T-DNA constructs or site specific excision of the selection gene by a specific recombinase.

In addition to direct transformation of a particular plant genotype with a construct of the present invention, transgenic plants may be made by crossing a plant having the construct to a second plant lacking the construct. For example, a construct encoding a variant can be introduced into a particular plant variety by crossing, without the need for ever directly transforming a plant of that given variety. Therefore, the present invention encompasses not only a plant directly regenerated from cells which have been transformed in accordance with the present invention, but also the progeny of such plants. As used herein, progeny may refer to the offspring of any generation of a parent plant prepared in accordance with the present invention. Such progeny may include a DNA construct prepared in accordance with the present invention. Crossing results in the introduction of a transgene into a plant line by cross pollinating a starting line with a donor plant line. Non-limiting examples of such steps are described in U.S. Pat. No. 7,151,204.

Plants may be generated through a process of backcross conversion. For example, plants include plants referred to as a backcross converted genotype, line, inbred, or hybrid.

Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

The present invention also relates to methods of producing a variant of the present invention comprising: (a) cultivating a transgenic plant or a plant cell comprising a polynucleotide encoding the variant under conditions conducive for production of the variant; and (b) recovering the variant.

The present invention is further described by the following examples that should not be construed as limiting the scope of the invention.

### EXAMPLES

### Materials

Chemicals used as buffers and substrates were commercial products of at least reagent grade.

### Media

1) pH 5.0 buffer with 50 mM acetate: 2.873 g sodium acetate and 0.901 g acetic acid were dissolved in 1 L de-ionized water;

2) pH 6.5 buffer with 50 mM phosphate: 5.642 g disodium hydrogen phosphate dodecahydrate (Na₂HPO₄·12H₂O) and 5.344 g sodium dihydrogen phosphate dehydrate(NaH₂PO₄·2H₂O) were dissolved in 1 L de-ionized water;

3) pH 7.5 buffer with 50 mM phosphate:15.045 g disodium hydrogen phosphate dodecahydrate (Na₂HPO₄·12H₂O) and 1.248 g sodium dihydrogen phosphate dehydrate(NaH₂PO₄·2H₂O) were dissolved in 1 L de-ionized water.

### Enzymes

| | | | | | |
|---|---|---|---|---|---|
| | | | | Mutations | |
| | | | | corresponding to | |
| | | | | O42EB3 mature | |
| Enzyme | Core | Linker | CBM | polypeptide | SEQ ID NOs |

Group 1

| | | | | | |
|---|---|---|---|---|---|
| O42EB3 | Tt cellulase | Sc cellulase | Sc cellulase | | SEQ ID NOs: 1 and 2 |
| O32JRC | Tt cellulase | Sc cellulase | Tt cellulase | | SEQ ID NOs: 3 and 4 |
| O62FA6 | Tt cellulase | Sc cellulase | Sc cellulase | W292Y | SEQ ID NOs: 5 and 6 |
| O62FA8 | Tt cellulase | Th cellulase | Th cellulase | R202A; | SEQ ID NOs: 7 and 8 |
| O62FAA | Tt cellulase | Th cellulase | Th cellulase | R202A; Y292W | SEQ ID NOs: 9 and 10 |

| | | |
|---|---|---|
| O52EWC | Thielavia terrestris GH45 cellulase | SEQ ID NOs: 37 and 12 |
| | (Tt cellulase), See SEQ ID | |
| | NOs: 3 and 4 in WO 2012/089024, | |
| | hereby incorporated by reference | |

Group 2

| O82AH2 | Staphylotrichum coccosporum GH45 cellulase | | | | SEQ ID NOs: 38 and 13 |
|---|---|---|---|---|---|
| | (Sc cellulase), See SEQ ID | | | | |
| | NOs 39 and 3 in WO2005/054475, | | | | |
| | hereby incorporated by reference | | | | |
| O82DT2 | Sc cellulase | | | W292Y | SEQ ID NOs 15 and 16 |
| O82DT4 | Sc cellulase | Tt cellulase | Tt cellulase | R202A | SEQ ID NOs 17 and 18 |

Group 3

| | | | |
|---|---|---|---|
| P24YEZ | Neurospora tetrasperma GH45 cellulase | | SEQ ID NOs 39 and 14 |
| | (Nt cellulase) See SEQ ID NO: 1 and | | |
| | SEQ ID NO: 2 in WO 2015/058700, | | |
| | hereby incorporated by reference | | |
| P446XD | Nt cellulase | Y292W | SEQ ID NOs 19 and 20 |

Group 4

| | | | | | |
|---|---|---|---|---|---|
| O14G83 | Tt cellulase | Sc cellulase | Sc cellulase | R265K + W266Y + | SEQ ID NOs 21 and 22 |
| | | | | W292Y | |
| O141FN | Tt cellulase | Sc cellulase | Sc cellulase | W266Y + F274Y + | SEQ ID NOs 23 and 24 |
| | | | | W292Y | |
| O141FS | Tt cellulase | Sc cellulase | Sc cellulase | T237N + T246N + | SEQ ID NOs 25 and 26 |
| | | | | W292Y | |
| O141FK | Tt cellulase | Sc cellulase | Sc cellulase | F274Y + W292Y | SEQ ID NOs 27 and 28 |
| O141FU | Tt cellulase | Sc cellulase | Sc cellulase | S255P + W292Y | SEQ ID NOs 29 and 30 |
| O141FF | Tt cellulase | Sc cellulase | Sc cellulase | W266Y + W292Y | SEQ ID NOs 31 and 32 |
| O141FH | Tt cellulase | Sc cellulase | Sc cellulase | R265K + W292Y | SEQ ID NOs 33 and 34 |
| O141FQ | Tt cellulase | Sc cellulase | Sc cellulase | S221P + S224P + | SEQ ID NOs 35 and 36 |
| | | | | W292Y | |

### Fabrics

Cotton interlock: 40S, bleached, HM-A0008, available from HM Cotton, Guangzhou, China.

Cotton single jersey: 40S/1AC+20D, bleached, Migen spandex plain, Sichuan, China.

Denim fabrics of basic and bamboo types and blue/brown knitted fabrics were purchased from Guangdong, China.

Navy fabrics dyed with reactive dyestuff were purchased from Esquel, Guangdong.

### Method

### Weight Loss Determination

The swatches were placed in the conditioned room (65%+/−5% humidity, 20+/−1° C.) for 24 hours before they were numbered, weighed by the analytical balance(for samples below 100 g) or a precision balance(for samples over 100 g) and recorded. After treatment, all samples were tumbled dried for 1 hour and conditioned for 24 hours in the conditioned room mentioned as above. For each sample, the weight loss was defined as below:

$$Weight loss U / 0 = \frac{( weight before treatment - weight after treatment ) * 100}{weight before treatment}$$

### Pilling Notes Test

Fabrics including treated and untreated which had been pre-conditioned in norm climate (65% humidity, 21° C.) for at least 24 hours were tested for the pilling notes with Nu-Martindale Tester (James H. Heal Co. Ltd, England), with untreated fabrics of the same type as the abraded fabrics. A standard pilling test (Swiss Norm (SN) 198525) was carried out after 2000 Revolutions by marking from 1-5, with the meaning defined as below, where 1 shows poor anti- pilling and 5 shows excellent anti-pilling property. Thus the higher the Martindale pilling notes score the more effective the endo-glucanase biopolishing treatment.
Note 5: No pilling
Note 4: Slight Pilling
Note 3: Moderate Pilling
Note 2: Distinct Pilling
Note 1: Heavy Pilling
½, ¼ notes are allowed

To make the test result more reliable, 3 separate readings were carried out by different persons for each sample, and the average of the 3 readings was adopted as the final result of pilling notes.

### Strength Determination

The fabric strength was tested with YGB031PC Electronic Marble Bursting Strength Tester according to GB/T19976-2005. Five parallel tests were included and the average was the calculated as the strength of the fabrics from each treatment.

### Color Measurement for Denim

The abrasion level and backstaining level of the denim samples were determined by measuring the reflectance with pre-calibrated DataColor SF450X, alternatively an equivalent apparatus can be used. Four readings were taken for each sample, and the average of the readings were used. The abrasion level was evaluated with the index CIE L* on the blue side (front side) of the sample, and the backstaining level was evaluated with the index CIE b* on the back side of the sample.

L* indicates the change in white/black on a scale from 0 to 100, and a decrease in L* means an increase in black colour (decrease in white colour) and an increase in L* means an increase in white colour (decrease in black colour). Delta L* unit=L* of the swatch treated with a certain celllulase−L* of the swatch before cellulase treatment. The larger the Delta L* unit is the higher is the denim abrasion level, e.g. a Delta L* unit of 4 has higher abrasion level than Delta L* unit of 3.

b* indicates the change in blue/yellow, and a decrease in b* means an increase in blue colour (decrease in yellow colour), and an increase in b* means an increase in yellow colour (decrease in blue colour). Delta b* units=b* of the swatch treated with a certain celllulase−b* of the swatch before cellulase treatment. A larger Delta b* unit corresponds to a lower backstaining level, e.g. a Delta b* unit of −1.5 has lower backstaining level than the Delta b* unit of −2.5.

### Protein Content

The enzyme protein in an enzyme product can be measured with BCA™ Protein Assay Kit (product number 23225, commercial available from Thermo Fisher Scientific Inc.) according to the product manual.

### Cellulase Activity Assay (CNUR/g)

The substrate carboxymethyl cellulose (CMC) was hydrolyzed with cellulase at pH 7.5, 50° C. for 30 min. The reac-tion is stopped by an alkaline reagent containing PAHBAH and bismuth which forms com-plexes with reducing sugar. The complex formation results in color production which can be read at 405 nm by a spectrophotometer. The produced color is proportional to the cellulase activity. Enzymatic reaction and measurement of absorbance proceed automati-cally in the Konelab analyzer. Cellulase activity is determined relative to a Novozymes enzyme standard. A detailed description of the assay, as well as a sample of the Renozyme™ standard, is available on request from Novozymes A/S, Krogshoejvej 36, DK-2880 Bagsvaerd, Denmark (EB-SM-0787.02-D).

### Example 1

### Construction of Cellulase Hybrid Genes by Attaching Different Linker/CBM to Tt Cel45a (CBM+) Core

The cellulase hybrid gene, RenoCorenew_ScLNK_ScCBM (SEQ ID NOs: 11 & 2), was generated by attaching the linker and CBM derived from Staphylotrichum coccosporum GH45 SWISSPROT:B5BNY1 to the c-terminus of core region of Tt Cel45a (CBM+) (WO2012/089024, hereby incorporated by reference). The plasmid of pRenoCBD described in WO2012/089024 was used as template for amplication of the core region of the hybrid gene. The linker and CBM region of Staphylotrichum coccosporum GH45 SWISSPROT:B5BNY1 was synthesized and cloned in ScLNKCBM_SynAO (SEQ ID NO: 40) by GENEWIZ Inc.

The following primers were synthesized by Invitrogen.

Primer 1

SEQ ID NO: 50

ACACAACTGGGGATC CACC atgcgctctactcccgttcttc
Primer 2

SEQ ID NO: 41

gctgcaagcgc aacgacgatggtaacttccctgtgttcacc
Primer 3

SEQ ID NO: 42

ggtgaacacagggaagttaccatc gtcgttgcgcttgcagc
Primer 4

SEQ ID NO: 43

CCCTCTAGATCTCGAG [symbol]
gagacactgggagtaccagtcgttc

The capitalized letters in Primer 1 represent the region homologous to insertion sites of pCaHj505 (WO2013/029496, hereby incorporated by reference). The underlined letters represent the Kozark sequence as the initiation of translation process. The lowercase letters represent the coding sequence of Tt Cel45a (CBM+). In Primer 2, regular letters represent the core sequence of Tt Cel45a (CBM+) and the italic letters represent the sequence of Staphylotrichum coccosporum GH45 linker. Primer 3 contains the complementary sequence of Primer 2. The capitalized letters in Primer 4 represent the region homologous to insertion sites of pCaHj505. The lowercase letters represent the complementary sequence of Staphylotrichum coccosporum GH45 CBM. The bold and underlined letters was the stop codon.

The splicing overlap extension PCR was performed to generate the hybrid gene. In 1St round of PCR, the core region and the linker/CBM region were amplified independently with details described in following.

For core region amplification, 20 pmol each of Primers 1 & 3 were used in PCR reaction composed of 2 μl of plasmid DNA of pRenoCBD, 10 μl of 5X Phusion® HF Buffer (Finnzymes Oy, Espoo, Finland), 1.5 μl of DMSO, 1.5 μl of 2.5 mM each of dATP, dTTP, dGTP, and dCTP, and 0.6 unit of PHUSION™ High-Fidelity DNA Polymerase (Finnzymes Oy, Espoo, Finland) in a final volume of 50 μl. The amplification was performed using a Peltier Thermal Cycler (M J Research Inc., South San Francisco, Calif., USA) programmed for denaturing at 98° C. for 1 minutes; 10 cycles of denaturing at 98° C. for 30 seconds, annealing at 65° C. for 30 seconds, with a 1° C. decrease per cycle and elongation at 72° C. for 70 seconds; 25 cycles each at 98° C. for 30 seconds, 60° C. for 30 seconds, and 72° C. for 70 seconds; and a final extension at 72° C. for 7 minutes. The heat block then went to a 4° C. soak cycle. The PCR product was isolated by 1.0% agarose gel electrophoresis using 90 mM Tris-borate and 1 mM EDTA (TBE) buffer where a single product band of approximately 0.8 kb from the reaction was visualized under UV light. The PCR product was then purified from solution by using an illustra™ GFX™ PCR DNA and Gel Band Purification Kit (GE Healthcare, Buckinghamshire, UK) according to the manufacturer's instructions. It was designated as Frag1.

For linker and CBM region amplification, 20 pmol each of Primers 2 & 4 were used in PCR reaction composed of 2 μl of ScLNKCBM_SynAO, 10 μl of 5X Phusion® HF Buffer, 1.5 μl of DMSO, 1.5 μl of 2.5 mM each of dATP, dTTP, dGTP, and dCTP, and 0.6 unit of PHUSION™ High-Fidelity DNA Polymerase in a final volume of 50 μl. The same program as Frag1 amplification was used. The purified PCR product was about 0.3 kb and designated as Frag2.

In 2^{nd} round of PCR, 20 pmol of Primers 1 & 4 were used in PCR reaction composed of 1 μl of Frag1 and 1 μl of Frag2, 10 μl of 5X Phusion® HF Buffer, 1.5 μl of DMSO, 1.5 μl of 2.5 mM each of dATP, dTTP, dGTP, and dCTP, and 0.6 unit of PHUSION™ High-Fidelity DNA Polymerase in a final volume of 50 μl. The same program as Frag1 amplification was used. A PCR product of about 1.1 kb was obtained. The purified PCR product was designated as Frag3.

Plasmid pCaHj505 was digested with Bam HI and Xho I, isolated by 1.0% agarose gel electrophoresis using TBE buffer, and purified using an ILLUSTRA™ GFX™ PCR DNA and Gel Band Purification Kit according to the manufacturer's instructions. In-Fusion® HD Cloning Kit (Clontech Laboratories, Inc., Mountain View, Calif., USA) was used to clone Frag3 directly into the expression vector pCaHj505, without the need for restriction digestion.

In details, 1 μl of 50 ng/μl of pCaHj505, digested with Bam HI and Xho I, and 3 μl of ddH₂O were added to In-Fusion Dry-Down Mix to get the pellet dissolved. Then 2 μl of the In-Fusion Dry-Down Mix solution was added to a new tube with addition of 3 μl of Frag3. The reaction was incubated at 50° C. for 15 minutes. The ligation reaction was used to transform E. coli TOP10 competent cells (TIANGEN Biotech Co. Ltd., Beijing, China). Three transformants were obtained and sent for sequence confirmation. The coding sequence of the cellulase hybrid gene, RenoCorenew_ScLNK_ScCBM, inserted in p505-RenoCorenew_ScLNK_ScCBM was confirmed by DNA sequencing using 3730XL DNA Analyzers (Applied Biosystems Inc, Foster City, Calif., USA). Thus the transcription of the cellulase hybrid gene was under the control of an Aspergillus oryzae alpha-amylase gene promoter. The plasmid of p505-RenoCorenew_ScLNK_ScCBM was prepared using a QIAPREP® Spin Miniprep Kit (QIAGEN GmbH, Hilden, Germany).

The 2^{nd} cellulase hybrid gene, RenoCorenew_ScLNK_RenoCBM (SEQ ID NOs: 3 & 4), was generated by replacing the CBM region of RenoCorenew_ScLNK_ScCBM with CBM derived from Tt Cel45a (CBM+). The plasmid of p505-RenoCorenew_ScLNK_ScCBM described above was used as template for amplification of the core and linker region. The plasmid pRenoCBD was used as template for amplification of CBM region.

The following primers were synthesized by Invitrogen.

Primer 5

SEQ ID NO: 44

cgtcgtccaccggagga ggctgcacgtctcagaagtg
Primer 6

SEQ ID NO: 45

cacttctgagacgtgcagcc tcctccggtggacgacg
Primer 7

SEQ ID NO: 46

CCCTCTAGATCTCGAG ggaaatcaaccagcagtcgc

In Primer 5, the regular letters represent the sequence of linker region of RenoCorenew_ScLNK_ScCBM and the italic letters represent the sequence of CBM region of Tt Cel45a(CBM+). Primer 6 contains the complementary sequence of Primer 5. The capitalized letters in Primer 7 represent the region homologous to insertion sites of pCaHj505. The italic lowercase letters were complemented to the downstream sequence of Tt Cel45a(CBM+) CDS

The splicing overlap extension PCR was performed to generate the hybrid gene.

For core and linker region amplification, Primer 1 & 6 were used as primer pair and p505-RenoCorenew_ScLNK_ScCBM as template to obtain PCR product Frag4 (about 1kb). The PCR program was as following: denaturing at 98° C. for 1 minutes; 8 cycles of denaturing each at 98° C. for 30 seconds, annealing at 67° C. for 30 seconds, with a 1° C. decrease per cycle and elongation at 72° C. for 60 seconds; 25 cycles each at 98° C. for 30 seconds, 60° C. for 30 seconds, and 72° C. for 60 seconds; and a final extension at 72° C. for 7 minutes.

For CBM region, Primer 5 & 7 were used as primer pair and pRenoCBD as template to obtain PCR product Frag5 (about 0.3kb). The same program as Frag1 amplification was used.

Then similar reaction as the one described in 2n^{d} round of PCR of RenoCorenew_ScLNK_ScCBM was made by using Primer 1 & 7 as a primer pair and Frag4 & 5 as template. The same program as Frag4 amplification was used. The resulted PCR fragment was about 1.3kb and designated as Frag6.

Frag6 was ligated to vector pCaHj505 with In-Fusion® HD Cloning Kit. The transformation in E. coli TOP10 resulted in several transformants. The transformant harbored the target gene was confirmed by DNA sequencing and designated as p505- RenoCorenew_ScLNK_RenoCBM). The plasmid was prepared for Aspergillus oryzae transformation in Example 2.

The coding sequence of the CBM region of RenoCorenew_ScLNK_ScCBM was further optimized as RenoCorenew_ScLNK_ScCBM_2 (SEQ ID NO: 1) for Aspergillus oryzae expression. ScCBM_2 (SEQ ID NO: 79) was synthesized in GENEWIZ Inc..

Primer 8

SEQ ID NO: 47

cgtcgtccaccggaggaggctgcgcagcgcagcg
Primer 9

SEQ ID NO: 48

tcctccggtggacgacg
Primer 10

SEQ ID NO: 49

CCCTCTAGATCTCGAG tcagaggcactgcgagtaccagtcg

The regular letters in Primer 8 represent the linker region of RenoCorenew_ScLNK_ScCBM and the italic letters represent the 5′ end of ScCBM_2. Primer 9 contains the complementary sequence of linker region. The captalized letters in Primer 10 represent the region homologous to insertion sites of pCaHj505 and the italic letters represent the 3′ end of ScCBM_2.

The plasmid DNA of p505-RenoCorenew_ScLNK_RenoCBM was used to amplify the core and linker region by using Primerl & 9 as primer pair. ScCBM_2 was used to amplify the CBM region by using primer8 & 10 as primer pair. The resulted PCR fragments, Frag7 and Frag8, were used for RenoCorenew_ScLNK_ScCBM_2 amplification by using Primerl & 10 as primer pair. The resulted PCR product, Frag9, was ligated to pCaHj505. The E. coli transformant harboring the target gene was confirmed by DNA sequencing. The plasmid DNA p505-RenoCorenew_ScLNK_ScCBM_2. was prepared for Aspergillus oryzae transformation in Example 2.

### Example 2

### Expression of the Cellulase Hybrid Genes in Aspergillus Oryzae

Aspergillus oryzae strain MT3568 was used for heterologous expression of the gene encoding the cellulase hybrid genes RenoCorenew_ScLNK_ScCBM_2 and RenoCorenew_ScLNK_RenoCBM. A. oryzae MT3568 is an amdS (acetamidase) disrupted gene derivative of A. oryzae JaL355 (WO 02/40694) in which pyrG auxotrophy was restored by disrupting the A. oryzae acetamidase (amdS) gene with the pyrG gene.

Protoplasts were prepared according to the method described as “Transformation of Aspergillus Expression Host” in Example 2 of US20140179588 A1. Three μg of plasmid DNA were used to transform Aspergillus oryzae MT3568.

The transformation of Aspergillus oryzae MT3568 with p505-RenoCorenew_ScLNK_ScCBM_2 or as p505-RenoCorenew_ScLNK_RenoCBM yielded about 20 transformants each. 3-4 transformants of each transformation were isolated to μlate for reisolation and were then inoculated separately into 3 ml of YPM medium in 24-well μlate and incubated at 30° C., 150 rpm. After 3 days incubation, 20 μl of supernatant from each culture were analyzed on NUPAGE® NOVEX® 4-12% Bis-Tris Gel with 50 mM MES (Invitrogen Corporation, Carlsbad, Calif., USA) according to the manufacturer's instructions. The resulting gel was stained with INSTANTBLUE™ (Expedeon Ltd., Babraham Cambridge, UK). SDS-PAGE profiles of the cultures showed that the majority of the transformants had a major band of approximately 55 kDa for both genes. Thus transformant #2 for RenoCorenew_ScLNK_ScCBM_2 and transformant #1 for RenoCorenew_ScLNK_RenoCBM were selected for shaking flask fermentation and designated as O42EB3 and O32JRC respectively.

### Example 3

Fermentation of Aspergillus oryzae Expression Strains O42EB3 and O32JRC

A slant of the expression strain O42EB3, was washed with 10 ml of YPM and inoculated into four 2-liter flasks each containing 400 ml of YPM medium (1% of Yeast extract, 2% of Peptone and 2% of Maltose), shaking at 30° C., 80 rpm. The culture was harvested on day 3 and filtered using a 0.45 μm DURAPORE Membrane (Millipore, Bedford, Mass., USA).

A slant of the expression strain O32JRC, was washed with 10 ml of YPM and inoculated into four 2-liter flasks each containing 400 ml of YPM medium, shaking at 30C, 80 rpm. The culture was harvested on day 3 and filtered using a 0.45 μm DURAPORE Membrane.

### Example 4

Purification and Activity Assay of Cellulase Hybrid from Aspergillus oryzae O42EB3 and O32JRC

A 1600 ml volume of filtered supernatant of Aspergillus oryzae O42EB3 (Example 3) was precipitated with ammonium sulfate (80% saturation), re-dissolved in 50 ml of 20 mM Tris-HCl pH 7.5, dialyzed against the same buffer, and filtered through a 0.45 μm filter. The final volume was 60 ml. The solution was applied to a 40 ml Q SEPHAROSE® Fast Flow column (GE Healthcare, Buckinghamshire, UK) equilibrated with 20 mM Tris-HCl pH 7.5. Proteins were eluted with a linear 0-0.25 M NaCl gradient. Unbound proteins were collected and further purified by using a MonoQ HR16/10 column (GE Healthcare, Buckinghamshire, UK) equilibrated with 20 mM Tris-HCl pH 7.5. Proteins were eluted with a linear 0-0.15 M NaCl gradient. Unbound proteins were collected and further purified by using a 40 ml SP SEPHAROSE® Fast Flow column (GE Healthcare, Buckinghamshire, UK) equilibrated with 20 mM NaAc pH 4.5. Proteins were eluted with a linear 0-0.25 M NaCl gradient. Fractions were analyzed by SDS-PAGE using a NUPAGE® NOVEX® 4-12% Bis-Tris Gel with 50 mM MES. The resulting gel was stained with INSTANTBLUE™. Fractions containing a band at approximately 55 kDa were pooled. Then the pooled solution was concentrated by ultrafiltration.

A 1600 ml volume of filtered supernatant of Aspergillus oryzae O32JRC was precipitated with ammonium sulfate (80% saturation), re-dissolved in 50 ml of 20 mM Tris-HCl pH 7.5, dialyzed against the same buffer, and filtered through a 0.45 μm filter. The final volume was 75 ml. The solution was applied to a 40 ml Avicel pH-101 column equilibrated with 20 mM Tris-HCl pH 7.5. Proteins were eluted with 1% triethylamine. Fractions eluted were collected and further purified using a 40 ml Q SEPHAROSE® Fast Flow column (GE Healthcare, Buckinghamshire, UK) equilibrated with 20 mM Tris-HCl pH 7.5. Proteins were eluted with a linear 0-0.5 M NaCl gradient. Fractions were analyzed by SDS-PAGE using a NUPAGE® NOVEX® 4-12% Bis-Tris Gel with 50 mM MES. The resulting gel was stained with INSTANTBLUE™. Fractions containing a band at approximately 55 kDa were pooled. Then the pooled solution was concentrated by ultrafiltration.

The cellulase activity was detected by blue substrate. The blue substrate of AZCL-HE-Cellulose (Megazyme) was dissolved into 100 mM Bis-Tris at pH6.0 with final concentration of 0.2% (w/v). 20 μl enzyme was mixed with 100 μl blue substrate and incubated at 50° C. for 30 min with 600 rpm by Thermomixer (Eppendorf), then 60 μl supernatant of mixture was transferred into a new well to measure absorbance at 595 nm. The cellulase hybrids showed good cellulase activity.

### Example 5

### Cloning of O62FA6 GH45 Cellulase

The GH45 cellulase gene, O62FA6 (SEQ ID NO: 5 for the DNA sequence and SEQ ID NO: 6 for the deduced amino acid sequence), was selected for expression cloning.

The oligonucleotide primers shown below were designed to amplify the full length GH45 cellulase gene from the plasmid p505-RenoCorenew_ScLNK_ScCBM_2 with a single substitution W292Y, corresponding to O42EB3 mature polypeptide. The primers were synthesized by Invitrogen, Beijing, China.

Forward primer D24H9NF (gene specific):

(SEQ ID NO: 50)

5′ ACACAACTGGGGATC CACC atgcgctctactcccgttcttc 3′
Reverse primer D24H9NR (mutagenic):

(SEQ ID NO: 51)

5′ CCCTCTAGATCTCGAG tcagaggcactgcgagtagtagtc 3′

Lowercase characters letters of the forward primer represent the coding region of the gene and lowercase characters letters of the reverse primer represent the flanking region of the gene, while bold characters letters represent a region homologous to insertion sites of pCaHj505 (WO2013/029496). The 4 letters ahead of the coding sequence in the forward primer D24H9NF represent the Kozak sequence as the initiation of translation process. The italic letters represent the nucleotides for the mutated amino acid.

Twenty picomoles of the forward and reverse primers above were used in a PCR reaction composed of 0.2 μl of p505-RenoCorenew_ScLNK_ScCBM_2 plasmid DNA, 10 μl of 5X Phusion® HF Buffer (Finnzymes Oy, Espoo, Finland), 1.5 μl of DMSO, 1.5 μl of 2.5 mM each of dATP, dTTP, dGTP, and dCTP, and 0.6 unit of Phusion® High-Fidelity DNA Polymerase (Finnzymes Oy, Espoo, Finland) in a final volume of 50 μl. The amplification was performed using a Peltier Thermal Cycler (M J Research Inc., South San Francisco, Calif., USA) programmed for denaturing at 98° C. for 1 minute; 10 cycles of denaturing each at 98° C. for 30 seconds, annealing at 65° C. for 30 seconds, with a 1° C. decrease per cycle and an elongation at 72° C. for 90 seconds; 25 cycles each at 98° C. for 30 seconds, 56° C. for 30 seconds, and 72° C. for 90 seconds; and a final extension at 72° C. for 7 minutes. The heat block then went to a 4° C. soak cycle.

The PCR product was isolated by 1.0% agarose gel electrophoresis using 90 mM Tris-borate and 1 mM EDTA (TBE) buffer where a single product band of approximately 1.1 kb from the reaction was visualized under UV light, and then it was purified using an ILLUSTRA™ GFX™ PCR DNA and Gel Band Purification Kit (GE Healthcare, Buckinghamshire, UK) according to the manufacturer's instructions.

Plasmid pCaHj505 was digested with Bam HI and Xho I, isolated by 1.0% agarose gel electrophoresis using TBE buffer, and purified using an ILLUSTRA™ GFX™ PCR DNA and Gel Band Purification Kit (GE Healthcare, Buckinghamshire, UK) according to the manufacturer's instructions.

In-Fusion HD Cloning Kit (Clontech Laboratories, Inc., Mountain View, Calif., USA) was used to clone the PCR fragment directly into the expression vector pCaHj505, without the need for restriction digestion and ligation. The purified PCR product and the digested vector were ligated together using the In-Fusion HD Cloning Kit according to the manufacturer's instructions resulting in a plasmid where the transcription of the 062FA6 GH45 cellulase polypeptide coding sequence was under the control of an Aspergillus oryzae alpha-amylase gene promoter. In brief, 1 μl of 30 ng/μl of pCaHj505, digested with Bam HI and Xho I, and 3 μl of the purified PCR product containing about 60 ng of the O62FA6 GH45 cellulase fragment were added to 1 μl of 5X In-Fusion HD Enzyme Premix. The reaction was incubated at 50° C. for 15 minutes, and then 5 μl of the ligation reaction were used to transform E. coli TOP10 competent cells (TIANGEN Biotech Co. Ltd., Beijing, China). E. coli transformants containing an expression construct were detected by colony PCR. Colony PCR is a method for quick screening of plasmid inserts directly from E. coli colonies. Briefly, a single colony was transferred to a premixed PCR solution in a PCR tube, including PCR buffer, MgCl₂, dNTPs, and primer pairs from which the PCR fragment was generated. Several colonies were screened. After the PCR, reactions were analyzed by 1.0% agarose gel electrophoresis using TBE buffer. Plasmid DNA was prepared using a QIAPREP® Spin Miniprep Kit (QIAGEN GmbH, Hilden, Germany) from the colony showing an insert with the expected size. The O62FA6 GH45 cellulase coding sequence inserted in pCaHj505 was confirmed by DNA sequencing using 3730XL DNA Analyzers (Applied Biosystems Inc, Foster City, Calif., USA).

The DNA sequence and deduced amino acid sequence of the O62FA6 mutant polypeptide coding sequence are shown in SEQ ID NO: 5 and SEQ ID NO: 6, respectively. The coding sequence is 1104 bp including the stop codon, which is interrupted by 2 introns of 78 bp (nucleotides 90 to 167) and 69 bp (nucleotides 417 to 485). The encoded protein is 318 amino acids with a signal peptide of 21 residues. The mature protein contains 297 amino acids with a predicted molecular mass of 30 kDa and a predicted isoelectric point of 5.0.

### Example 6

### Cloning of O62FA8 and O62FAA GH45 Cellulases

The GH45 cellulase genes, O62FA8 (SEQ ID NO: 7 for the DNA sequence and SEQ ID NO: 8 for the deduced amino acid sequence) and O62FAA (SEQ ID NO: 9 for the DNA sequence and SEQ ID NO: 10 for the deduced amino acid sequence), were selected for expression cloning.

For O62FA8, the oligonucleotide primers shown below were designed to separately amplify the GH45 cellulase core from the plasmid p505-RenoCorenew_ScLNK_ScCBM_2 and the linker/CBM domain from the plasmid pGH45_Thihy3331 (WO 2014/101753, hereby incorporated by reference) with a single substitution R202A, corresponding to 042EB3 mature polypeptide. The primers were synthesized by Invitrogen, Beijing, China.

Forward primer D24H9NF:

(SEQ ID NO: 50)

5′ ACACAACTGGGGATC CACC atgcgctctactcccgttcttc 3′
Reverse primer D24H9TR:

(SEQ ID NO: 52)

5′ agctggagtcgtcgtt cgccttgcagccgg 3′
Forward primer D24H9TlinkerF:

(SEQ ID NO: 53)

5′ ctccggctgcaaggcg aacgacgactccagcttccc 3′
Reverse primer D24H9TlinkerR:

(SEQ ID NO: 54)

5′ CCCTCTAGATCTCGAG ttagaggcactgcgagtagt
agtcg 3′

The capitalized characters are a region homologous to the insertion sites of pCaHj505 (WO2013/029496). The lowercase characters represent coding region of the Thielavia terrestris GH45 cellulase (Tt cellulase) core, and the bold characters are the coding region of Thielavia hyrcaniae GH45 endoglucanase (Th cellulase) linker/CBM domain. The 4 letters ahead of the coding sequence in forward primer D24H9NF represent the Kozak sequence as the initiation of translation process.

Forward primer D24H9NF and reverse primer D24H9TR were used to amplify the GH45 cellulase core from 042EB3. Twenty picomoles of the forward and reverse primers above were used in a PCR reaction composed of 0.2 μl of p505-RenoCorenew_ScLNK_ScCBM_2 plasmid DNA, 10 μl of 5X Phusion® HF Buffer (Finnzymes Oy, Espoo, Finland), 1.5 μl of DMSO, 1.5 μl of 2.5 mM each of dATP, dTTP, dGTP, and dCTP, and 0.6 unit of Phusion® High-Fidelity DNA Polymerase (Finnzymes Oy, Espoo, Finland) in a final volume of 50 μl. The amplification was performed using a Peltier Thermal Cycler (MJ Research Inc., South San Francisco, Calif., USA) programmed for denaturing at 98° C. for 1 minute; 10 cycles of denaturing each at 98° C. for 30 seconds, annealing at 65° C. for 30 seconds, with a 1° C. decrease per cycle and an elongation at 72° C. for 90 seconds; 25 cycles each at 98° C. for 30 seconds, 56° C. for 30 seconds, and 72° C. for 90 seconds; and a final extension at 72° C. for 7 minutes. The heat block then went to a 4° C. soak cycle.

The PCR product was isolated by 1.0% agarose gel electrophoresis using 90 mM Tris-borate and 1 mM EDTA (TBE) buffer where a single product band of approximately 816 bp from the reaction was visualized under UV light, and then it was purified using an ILLUSTRA™ GFX™ PCR DNA and Gel Band Purification Kit (GE Healthcare, Buckinghamshire, UK) according to the manufacturer's instructions. The PCR product band was designated as F3.

Forward primer D24H9TlinkerF and reverse primer D24H9TlinkerR were used to amplify the linker/CBM domain from Th cellulase. Twenty picomoles of the forward and reverse primers above were used in a PCR reaction composed of 0.2 μl of pGH45_Thihy3331 plasmid DNA, 10 μl of 5X Phusion® HF Buffer (Finnzymes Oy, Espoo, Finland), 1.5 μl of DMSO, 1.5 μl of 2.5 mM each of dATP, dTTP, dGTP, and dCTP, and 0.6 unit of Phusion® High-Fidelity DNA Polymerase (Finnzymes Oy, Espoo, Finland) in a final volume of 50 μl. The amplification was performed using a Peltier Thermal Cycler (MJ Research Inc., South San Francisco, Calif., USA) programmed for denaturing at 98° C. for 1 minute; 10 cycles of denaturing each at 98° C. for 30 seconds, annealing at 65° C. for 30 seconds, with a 1° C. decrease per cycle and an elongation at 72° C. for 90 seconds; 25 cycles each at 98° C. for 30 seconds, 56° C. for 30 seconds, and 72° C. for 90 seconds; and a final extension at 72° C. for 7 minutes. The heat block then went to a 4° C. soak cycle.

The PCR product was isolated by 1.0% agarose gel electrophoresis using 90 mM Tris-borate borate and 1 mM EDTA (TBE) buffer where a single product band of approximately 255 bp from the reaction was visualized under UV light, and then it was purified using an ILLUSTRA™ M GFX™ TM PCR DNA and Gel Band Purification Kit (GE Healthcare, Buckinghamshire, UK) according to the manufacturer's instructions. The PCR product band was designated as F4.

Plasmid pCaHj505 was digested with Bam HI and Xho I, isolated by 1.0% agarose gel electrophoresis using TBE buffer, and purified using an ILLUSTRA™ GFX™ PCR DNA and Gel Band Purification Kit (GE Healthcare, Buckinghamshire, UK) according to the manufacturer's instructions.

In-Fusion HD Cloning Kit (Clontech Laboratories, Inc., Mountain View, Calif., USA) was used to clone the PCR fragments directly into the expression vector pCaHj505, without the need for restriction digestion and ligation. The purified PCR products and the digested vector were ligated together using the In-Fusion HD Cloning Kit according to the manufacturer's instructions resulting in a plasmid where the transcription of the O62FA8 GH45 cellulase polypeptide coding sequence was under the control of an Aspergillus oryzae alpha-amylase gene promoter. In brief, 0.5 μl of 30 ng/μl of pCaHj505, digested with Bam HI and Xho I, 1.5 μl of the F3 PCR product and 0.6 μl of the F4 PCR product, were added to 1 μl of 5X In-Fusion HD Enzyme Premix in a final volume of 5 μl. The molar ratio of PCR fragments was 1:1, and the vector: insert ratio was 1:5. The reaction was incubated at 50° C. for 15 minutes, and then 5 μl of the ligation reaction were used to transform E. coli TOP10 competent cells (TIANGEN Biotech Co. Ltd., Beijing, China). E. coli transformants containing an expression construct were detected by colony PCR. Colony PCR is a method for quick screening of plasmid inserts directly from E. coli colonies. Briefly, a single colony was transferred to a premixed PCR solution in a PCR tube, including PCR buffer, MgCl₂, dNTPs, and primer pairs from which the PCR fragment was generated. Several colonies were screened. After the PCR, reactions were analyzed by 1.0% agarose gel electrophoresis using TBE buffer. Plasmid DNA was prepared using a QIAPREP® Spin Miniprep Kit (QIAGEN GmbH, Hilden, Germany) from the colony showing an insert with the expected size. The O62FA8 GH45 cellulase coding sequence inserted in pCaHj505 was confirmed by DNA sequencing using 3730XL DNA Analyzers (Applied Biosystems Inc, Foster City, Calif., USA).

The DNA sequence and deduced amino acid sequence of the O62FA8 mutant polypeptide coding sequence are shown in SEQ ID NO: 7 and SEQ ID NO: 8, respectively. The coding sequence is 1071 bp including the stop codon, which is interrupted by 2 introns of 78 bp (nucleotides 90 to 167) and 69 bp (nucleotides 417 to 485). The encoded protein is 307 amino acids with a signal peptide of 21 residues. The mature protein contains 286 amino acids with a predicted molecular mass of 29 kDa and a predicted isoelectric point of 5.8.

For O62FAA, the oligonucleotide primers shown below were designed to separately amplify the GH45 cellulase core from the plasmid p505-RenoCorenew_ScLNK_ScCBM_2 and the linker/CBM domain from the plasmid pGH45_Thihy3331, with the single substitutions R202A and Y292W, corresponding to O42EB3 mature polypeptide. The primers were synthesized by Invitrogen, Beijing, China.

Forward primer D24H9NF:

(SEQ ID NO: 50)

5′ ACACAACTGGGGATC CACC atgcgctctactcccgttcttc 3′
Reverse primer D24H9TR:

(SEQ ID NO: 52)

5′ agctggagtcgtcgtt cgccttgcagccgg 3′
Forward primer D24H9TlinkerF:

(SEQ ID NO: 53)

5′ ctccggctgcaaggcg aacgacgactccagcttccc 3′
Reverse primer D24H9XlinkerR:

(SEQ ID NO: 55)

5′ CCCTCTAGATCTCGAG
ttagaggcactgcgagta [symbol] gtc 3′

The capitalized characters are a region homologous to the insertion sites of pCaHj505 (WO2013/029496). The lowercase characters represent coding region of the Tt cellulase core, and the bold characters are the coding region of Th cellulase linker/CBM domain. The 4 letters ahead of the coding sequence in forward primer D24H9NF represent the Kozak sequence as the initiation of translation process.

Two PCR fragments of 816 bp and 255 bp were individually amplified and purified, and then cloned directly into the expression vector pCaHj505 as described for 062FA8. The DNA sequence and deduced amino acid sequence of the O62FAA mutant polypeptide coding sequence are shown in SEQ ID NO: 9 and SEQ ID NO: 10, respectively. The coding sequence is 1071 bp including the stop codon, which is interrupted by 2 introns of 78 bp (nucleotides 90 to 167) and 69 bp (nucleotides 417 to 485). The encoded protein is 307 amino acids with a signal peptide of 21 residues. The mature protein contains 286 amino acids with a predicted molecular mass of 29 kDa and a predicted isoelectric point of 5.8.

### Example 7

### Cloning of O82AH2, O82DT2 and O82DT4 GH45 Cellulases

The GH45 cellulase genes, O82AH2 (SEQ ID NO: 38 for the DNA sequence and SEQ ID NO: 13 for the deduced amino acid sequence), O82DT2 (SEQ ID NO: 15 for the DNA sequence and SEQ ID NO: 16 for the deduced amino acid sequence) and O82DT4 (SEQ ID NO: 17 for the DNA sequence and SEQ ID NO: 18 for the deduced amino acid sequence) were selected for expression cloning.

For O82AH2, the oligonucleotide primers shown below were designed to amplify the full length GH45 cellulase gene, from the plasmid D24YEA (SEQ ID NO: 38), synthesized (Genewiz, Suzhou, China) based on the sequence information obtained from SEQ ID NO: 3 in WO2005/054475. The primers were synthesized by Invitrogen, Beijing, China.

Forward primer D24YEAF:

(SEQ ID NO: 56)

5′ ACACAACTGGGGATC CACC atgcgctcctcccctg 3′
Reverse primer D24YEAR:

(SEQ ID NO: 57)

5′ CCCTCTAGATCTCGAG ttagagacactgggagtaccagtcg 3′

The capitalized letters are a region homologous to the insertion sites of pCaHj505 (WO2013/029496), and the lower case letters represent coding region of the O82AH2 GH45 cellulase. The 4 letters ahead of the coding sequence in forward primer D24YEAF represent the Kozak sequence as the initiation of translation process.

A PCR fragment of 951 bp was amplified, purified and then cloned directly into the expression vector pCaHj505 as described for O62FA6 in Example 5. The DNA sequence and deduced amino acid sequence of the O82AH2 polypeptide coding sequence are shown in SEQ ID NO: 38 and SEQ ID NO: 13, respectively. The coding sequence is 951 bp including the stop codon, with no introns. The encoded protein is 316 amino acids with a signal peptide of 21 residues. The mature protein contains 295 amino acids with a predicted molecular mass of 31 kDa and a predicted isoelectric point of 7.3.

For O82DT2, the oligonucleotide primers shown below were designed to amplify the full length GH45 cellulase gene from the plasmid D24YEA, with a single substitution W292Y corresponding to O42EB3 mature polypeptide. The primers were synthesized by Invitrogen, Beijing, China.

Forward primer D24YEAF:

(SEQ ID NO: 56)

5′ ACACAACTGGGGATC CACC atgcgctcctcccctg 3′
Reverse primer D347FKR:

(SEQ ID NO: 58)

5′ CCCTCTAGATCTCGAG ttagagacactgggagtagtagtcgttc
3′ (The italic letters represent the nucleotides
for the mutated amino acid)

The capitalized letters are a region homologous to the insertion sites of pCaHj505 (WO2013/029496), and the lower case letters represent coding region of the O82DT2 GH45 cellulase. The 4 letters ahead of the coding sequence in forward primer D24YEAF represent the Kozak sequence as the initiation of translation process. The italic letters represent the nucleotides for the mutated amino acid.

A PCR fragment of 951 bp was amplified, purified and then cloned directly into the expression vector pCaHj505 as described for O62FA6 in Example 1. The DNA sequence and deduced amino acid sequence of the O82DT2 mutant polypeptide coding sequence are shown in SEQ ID NO: 15 and SEQ ID NO: 16, respectively. The coding sequence is 951 bp including the stop codon, with no introns. The encoded protein is 316 amino acids with a signal peptide of 21 residues. The mature protein contains 295 amino acids with a predicted molecular mass of 31 kDa and a predicted isoelectric point of 7.3.

For O82DT4, the oligonucleotide primers shown below were designed to separately amplify the GH45 cellulase core from the plasmid D24YEA and the linker/CBM domain from Plasmid1 (WO2012/089024, hereby incorporated by reference), with a single substitution R202A corresponding to O42EB3 mature polypeptide. The primers were synthesized by Invitrogen, Beijing, China.

Forward primer D24YEAF:

(SEQ ID NO: 56)

5′ ACACAACTGGGGATC CACC atgcgctcctcccctg 3′
Reverse primer D3379PlinkerR:

(SEQ ID NO: 59)

5′ tggagtcgtcgtt tgcgcgacagccggtcctg 3′
Forward primer D3379PlinkerF:

(SEQ ID NO: 60)

5′ gaccggctgtcgcgca aacgacgactccagcttccc 3′
Reverse primer D24H9TlinkerR:

(SEQ ID NO: 54)

5′ CCCTCTAGATCTCGAG
ttagaggcactgcgagtagtagtcg 3′

The capitalized letters are a region homologous to the insertion sites of pCaHj505 (WO2013/029496). The lowercase letters represent coding region of the Staphylotrichum coccosporum GH45 cellulase (Sc cellulase) core, and the bold letters are the coding region of Thielavia terrestris GH45 cellulase (Tt cellulase) linker/CBM domain. The 4 letters ahead of the coding sequence in forward primer D24YEAF represent the Kozak sequence as the initiation of translation process.

Two PCR fragments of 663 bp and 231 bp were individually amplified and purified, and then cloned directly into the expression vector pCaHj505 as described for O62FA8. The DNA sequence and deduced amino acid sequence of the O82DT4 mutant polypeptide coding sequence are shown in SEQ ID NO: 17 and SEQ ID NO: 18, respectively. The coding sequence is 894 bp including the stop codon, with no introns. The encoded protein is 297 amino acids with a signal peptide of 21 residues. The mature protein contains 276 amino acids with a predicted molecular mass of 28 kDa and a predicted isoelectric point of 5.2.

### Example 8

### Cloning of P446XD GH45 Cellulase

The GH45 cellulase gene, P446XD (SEQ ID NO: 19 for the DNA sequence and SEQ ID NO: 20 for the deduced amino acid sequence), was selected for expression cloning.

The oligonucleotide primers shown below were designed to amplify the full length GH45 cellulase gene from the plasmid 001-8#1 (comprising Neurospora tetrasperma cellulase, SEQ ID NO: 2 in WO 2015/058700, hereby incorporated by reference), with the single substitution Y292W corresponding to O42EB3 mature polypeptide. The primers were synthesized by Invitrogen, Beijing, China.

Forward primer D43NXPF (gene specific):

(SEQ ID NO: 61)

5′ ACACAACTGGGGATC CACC atgcgctcctccactgttc 3′
Reverse primer D43NXPR (mutagenic):

(SEQ ID NO: 62)

5′ CCCTCTAGATCTCGAG ttaggcacactggtggtaccaatc
3′ (The italic letters represent the nucleotides
for the mutated amino acid)

The capitalized letters are a region homologous to the insertion sites of pCaHj505 (WO2013/029496), and the lower case letters represent coding region of the P446XD GH45 cellulase. The 4 letters ahead of the coding sequence in forward primer D43NXPF represent the Kozak sequence as the initiation of translation process. The italic letters represent the nucleotides for the mutated amino acid.

A PCR fragment of 935 bp was amplified, purified and then cloned directly into the expression vector pCaHj505 as described for O62FA6. The DNA sequence and deduced amino acid sequence of the P446XD mutant polypeptide coding sequence are shown in SEQ ID NO: 19 and SEQ ID NO: 20, respectively. The coding sequence is 935 bp including the stop codon, which is interrupted by 1 intron of 53 bp (nucleotides 339 to 391). The encoded protein is 293 amino acids with a signal peptide of 21 residues. The mature protein contains 272 amino acids with a predicted molecular mass of 28 kDa and a predicted isoelectric point of 6.9.

### Example 9

### Cloning of O62FA6 Variants (O14G83, O141FN, O141FS, O141FK, O141FU, O141FF, O141FH and O141FQ)

Eight variants of O62FA6 were selected for expression cloning, including O14G83 (SEQ ID NO: 21 for the DNA sequence and SEQ ID NO: 22 for the deduced amino acid sequence), O141FN (SEQ ID NO: 23 for the DNA sequence and SEQ ID NO: 24 for the deduced amino acid sequence), O141FS (SEQ ID NO: 25 for the DNA sequence and SEQ ID NO: 26 for the deduced amino acid sequence), O141FK (SEQ ID NO: 27 for the DNA sequence and SEQ ID NO: 28 for the deduced amino acid sequence), O141FU (SEQ ID NO: 29 for the DNA sequence and SEQ ID NO: 30 for the deduced amino acid sequence), O141FF (SEQ ID NO: 31 for the DNA sequence and SEQ ID NO: 32 for the deduced amino acid sequence), O141FH (SEQ ID NO: 33 for the DNA sequence and SEQ ID NO: 34 for the deduced amino acid sequence) and O141FQ (SEQ ID NO: 35 for the DNA sequence and SEQ ID NO: 36 for the deduced amino acid sequence).

For O14G83, the oligonucleotide primers shown below were designed to amplify the full length GH45 cellulase gene from the plasmid D24H9N for O62FA6 (SEQ ID NO: 5), with two substitutions R265K and W266Y corresponding to O42EB3 mature polypeptide. The primers were synthesized by Invitrogen, Beijing, China.

Forward primer D24H9NF:

(SEQ ID NO: 50)

5′ ACACAACTGGGGATC CACC atgcgctctactcccgttcttc 3′
Reverse primer D43NG3linkerRnew:

(SEQ ID NO: 63)

5′ cgcactgagcgtactt ctgcgctgcgcagc 3′
Forward primer D43NG3linkerFnew:

(SEQ ID NO: 64)

5′ aagtacgctcagtgcggtgg 3′
Reverse primer D24H9NR:

(SEQ ID NO: 51)

5′ CCCTCTAGATCTCGAG tcagaggcactgcgagtagtagtc 3′

The capitalized letters are a region homologous to the insertion sites of pCaHj505 (WO2013/029496), and the lower case letters represent coding region of the O14G83 GH45 cellulase. The 4 letters ahead of the coding sequence in forward primer D24H9NF represent the Kozak sequence as the initiation of translation process. The italic letters represent the nucleotides for the mutated amino acids.

Two PCR fragments of 1018 bp and 102 bp were individually amplified and purified, and then cloned directly into the expression vector pCaHj505 as described for O62FA8. The DNA sequence and deduced amino acid sequence of the O14G83 mutant polypeptide coding sequence are shown in SEQ ID NO: 21 and SEQ ID NO: 22, respectively. The coding sequence is 1104 bp including the stop codon, which is interrupted by 2 introns of 78 bp (nucleotides 90 to 167) and 69 bp (nucleotides 417 to 485). The encoded protein is 318 amino acids with a signal peptide of 21 residues. The mature protein contains 297 amino acids with a predicted molecular mass of 30 kDa and a predicted isoelectric point of 5.8.

For O141FN, the oligonucleotide primers shown below were designed to amplify the full length GH45 cellulase gene from the plasmid D24H9N for 062FA6 (SEQ ID NO: 5), with two substitutions, W265Y and F274Y corresponding to O42EB3 mature polypeptide. The primers were synthesized by Invitrogen, Beijing, China.

Forward primer D24H9NF:

(SEQ ID NO: 50)

5′ ACACAACTGGGGATC CACC atgcgctctactcccgttcttc 3′
Reverse primer D43NJ3linkerR:

(SEQ ID NO: 65)

5′ gctgtagccgatgcca ccgcactgagcgtaacgc 3′
Forward primer D43NJ3linkerF:

(SEQ ID NO: 66)

5′ gcgttacgctcagtgc ggtggcatcggctacagc 3′
Reverse primer D24H9NR:

(SEQ ID NO: 51)

5′ CCCTCTAGATCTCGAG tcagaggcactgcgagtagtagtc 3′

The capitalized letters are a region homologous to the insertion sites of pCaHj505 (WO2013/029496), and the lower case letters represent coding region of the O141FN GH45 cellulase. The 4 letters ahead of the coding sequence in forward primer D24H9NF represent the Kozak sequence as the initiation of translation process. The italic letters represent the nucleotides for the mutated amino acids.

Two PCR fragments of 1035 bp and 103 bp were individually amplified and purified, and then cloned directly into the expression vector pCaHj505 as described for O62FA8. The DNA sequence and deduced amino acid sequence of the O141FN mutant polypeptide coding sequence are shown in SEQ ID NO: 23 and SEQ ID NO: 24, respectively. The coding sequence is 1104 bp including the stop codon, which is interrupted by 2 introns of 78 bp (nucleotides 90 to 167) and 69 bp (nucleotides 417 to 485). The encoded protein is 318 amino acids with a signal peptide of 21 residues. The mature protein contains 297 amino acids with a predicted molecular mass of 30 kDa and a predicted isoelectric point of 5.8.

For O141FS, the oligonucleotide primers shown below were designed to amplify the full length GH45 cellulase gene from the plasmid D24H9N for O62FA6 (SEQ ID NO: 5), with two substitutions, T237N and T246N corresponding to O42EB3 mature polypeptide. The primers were synthesized by Invitrogen, Beijing, China.

Forward primer D24H9NF:

(SEQ ID NO: 50)

5′ ACACAACTGGGGATC CACC atgcgctctactcccgttcttc 3′
Reverse primer D43PNTlinkerR:

(SEQ ID NO: 67)

5′ ggagtttgtggaggtt gctttggtcgaagtgttggac 3′
Forward primer D43PNTlinkerF:

(SEQ ID NO: 68)

5′ cgtccaacacttcgac caaagcaacctccacaaactcc 3′
Reverse primer D24H9NR:

(SEQ ID NO: 51)

5′ CCCTCTAGATCTCGAG tcagaggcactgcgagtagtagtc 3′

The capitalized letters are a region homologous to the insertion sites of pCaHj505 (WO2013/029496), and the lower case letters represent coding region of the O141FS GH45 cellulase. The 4 letters ahead of the coding sequence in forward primer D24H9NF represent the Kozak sequence as the initiation of translation process. The italic letters represent the nucleotides for the mutated amino acids.

Two PCR fragments of 951 bp and 191 bp were individually amplified and purified, and then cloned directly into the expression vector pCaHj505 as described for O62FA8. The DNA sequence and deduced amino acid sequence of the O141FS mutant polypeptide coding sequence are shown in SEQ ID NO: 25 and SEQ ID NO: 26, respectively. The coding sequence is 1104 bp including the stop codon, which is interrupted by 2 introns of 78 bp (nucleotides 90 to 167) and 69 bp (nucleotides 417 to 485). The encoded protein is 318 amino acids with a signal peptide of 21 residues. The mature protein contains 297 amino acids with a predicted molecular mass of 30 kDa and a predicted isoelectric point of 5.8.

For O141FK, the oligonucleotide primers shown below were designed to amplify the full length GH45 cellulase gene from the plasmid D24H9N (SEQ ID NO: 5) with a single substitution F274Y. The primers were synthesized by Invitrogen, Beijing, China.

Forward primer D24H9NF:

(SEQ ID NO: 50)

5′ ACACAACTGGGGATC CACC atgcgctctactcccgttcttc 3′
Reverse primer D43NHUlinkerR:

(SEQ ID NO: 69)

5′ gctgtagccgatgcca ccgcactgagcccaac 3′
Forward primer D43NHUlinkerF:

(SEQ ID NO: 70)

5′ gcgttgggctcagtgc ggtggcatcggctacagc 3′
Reverse primer D24H9NR:

(SEQ ID NO: 51)

5′ CCCTCTAGATCTCGAG tcagaggcactgcgagtagtagtc 3′

The capitalized letters are a region homologous to the insertion sites of pCaHj505 (WO2013/029496), and the lower case letters represent coding region of the O141FK GH45 cellulase. The 4 letters ahead of the coding sequence in forward primer D24H9NF represent the Kozak sequence as the initiation of translation process. The italic letters represent the nucleotides for the mutated amino acid.

Two PCR fragments of 1035 bp and 103 bp were individually amplified and purified, and then cloned directly into the expression vector pCaHj505 as described for O62FA8. The DNA sequence and deduced amino acid sequence of the O141FK mutant polypeptide coding sequence are shown in SEQ ID NO: 27 and SEQ ID NO: 28, respectively. The coding sequence is 1104 bp including the stop codon, which is interrupted by 2 introns of 78 bp (nucleotides 90 to 167) and 69 bp (nucleotides 417 to 485). The encoded protein is 318 amino acids with a signal peptide of 21 residues. The mature protein contains 297 amino acids with a predicted molecular mass of 30 kDa and a predicted isoelectric point of 5.8.

For O141FU, the oligonucleotide primers shown below were designed to amplify the full length GH45 cellulase gene from the plasmid D24H9N for O62FA6 (SEQ ID NO: 5), with a single substitution S255P corresponding to O42EB3 mature polypeptide. The primers were synthesized by Invitrogen, Beijing, China.

Forward primer D24H9NF:

(SEQ ID NO: 50)

5′ ACACAACTGGGGATC CACC atgcgctctactcccgttcttc 3′
Reverse primer D43PNNlinkerR:

(SEQ ID NO: 71)

5′ ggacggcgacgtctgg gacgaggcggtggaagttg 3′
Forward primer D43PNNlinkerF:

(SEQ ID NO: 72)

5′ cttccaccgcctcgtc ccagacgtcgccgtcc 3′
Reverse primer D24H9NR:

(SEQ ID NO: 51

5′ CCCTCTAGATCTCGAG tcagaggcactgcgagtagtagtc 3′

The capitalized letters are a region homologous to the insertion sites of pCaHj505 (WO2013/029496), and the lower case letters represent coding region of the O141FU GH45 cellulase. The 4 letters ahead of the coding sequence in forward primer D24H9NF represent the Kozak sequence as the initiation of translation process. The italic letters represent the nucleotides for the mutated amino acid.

Two PCR fragments of 978 bp and 158 bp were individually amplified and purified, and then cloned directly into the expression vector pCaHj505 as described for O62FA8. The DNA sequence and deduced amino acid sequence of the O141FU mutant polypeptide coding sequence are shown in SEQ ID NO: 29 and SEQ ID NO: 30, respectively. The coding sequence is 1104 bp including the stop codon, which is interrupted by 2 introns of 78 bp (nucleotides 90 to 167) and 69 bp (nucleotides 417 to 485). The encoded protein is 318 amino acids with a signal peptide of 21 residues. The mature protein contains 297 amino acids with a predicted molecular mass of 30 kDa and a predicted isoelectric point of 5.8.

For O141FF, the oligonucleotide primers shown below were designed to amplify the full length GH45 cellulase gene from the plasmid D24H9N for O62FA6 (SEQ ID NO: 5), with a single substitution W266Y corresponding to O42EB3 mature polypeptide. The primers were synthesized by Invitrogen, Beijing, China.

Forward primer D24H9NF:

(SEQ ID NO: 50)

5′ ACACAACTGGGGATC CACC atgcgctctactcccgttcttc 3′
Reverse primer D43NFYlinkerR:

(SEQ ID NO: 73)

5′ agcgtaacgctgcgct gcgcagcctcctccg 3′
Forward primer D43NFYlinkerF:

(SEQ ID NO: 74)

5′ ccggaggaggctgcgc agcgcagcgttacgctc 3′
Reverse primer D24H9NR:

(SEQ ID NO: 51)

5′ CCCTCTAGATCTCGAG tcagaggcactgcgagtagtagtc 3′

The capitalized letters are a region homologous to the insertion sites of pCaHj505 (WO2013/029496), and the lower case letters represent coding region of the O141FF GH45 cellulase. The 4 letters ahead of the coding sequence in forward primer D24H9NF represent the Kozak sequence as the initiation of translation process. The italic letters represent the nucleotides for the mutated amino acid.

Two PCR fragments of 1011 bp and 125 bp were individually amplified and purified, and then cloned directly into the expression vector pCaHj505 as described for O62FA8. The DNA sequence and deduced amino acid sequence of the O141FF mutant polypeptide coding sequence are shown in SEQ ID NO: 31 and SEQ ID NO: 32, respectively. The coding sequence is 1104 bp including the stop codon, which is interrupted by 2 introns of 78 bp (nucleotides 90 to 167) and 69 bp (nucleotides 417 to 485). The encoded protein is 318 amino acids with a signal peptide of 21 residues. The mature protein contains 297 amino acids with a predicted molecular mass of 30 kDa and a predicted isoelectric point of 5.8.

For O141FH, the oligonucleotide primers shown below were designed to amplify the full length GH45 cellulase gene from the plasmid D24H9N for O62FA6 (SEQ ID NO: 5), with a single substitution R265K corresponding to O42EB3 mature polypeptide. The primers were synthesized by Invitrogen, Beijing, China.

Forward primer D24H9NF:

(SEQ ID NO: 50)

5′ ACACAACTGGGGATC CACC atgcgctctactcccgttcttc 3′
Reverse primer D43NG1linkerR:

(SEQ ID NO: 75)

5′ ccacttctgcgctgcg cagcctcctccggtgg 3′
Forward primer D43NG1linkerF:

(SEQ ID NO: 76)

5′ ccaccggaggaggctg cgcagcgcagaagtgg 3′
Reverse primer D24H9NR:

(SEQ ID NO: 51)

5′ CCCTCTAGATCTCGAG tcagaggcactgcgagtagtagtc 3′

The capitalized letters are a region homologous to the insertion sites of pCaHj505 (WO2013/029496), and the lower case letters represent coding region of the O141FH GH45 cellulase. The 4 letters ahead of the coding sequence in forward primer D24H9NF represent the Kozak sequence as the initiation of translation process. The italic letters represent the nucleotides for the mutated amino acid.

Two PCR fragments of 1008 bp and 128 bp were individually amplified and purified, and then cloned directly into the expression vector pCaHj505 as described for O62FA8. The DNA sequence and deduced amino acid sequence of the O141FH mutant polypeptide coding sequence are shown in SEQ ID NO: 33 and SEQ ID NO: 34, respectively. The coding sequence is 1104 bp including the stop codon, which is interrupted by 2 introns of 78 bp (nucleotides 90 to 167) and 69 bp (nucleotides 417 to 485). The encoded protein is 318 amino acids with a signal peptide of 21 residues. The mature protein contains 297 amino acids with a predicted molecular mass of 30 kDa and a predicted isoelectric point of 5.8.

For O141FQ, the oligonucleotide primers shown below were designed to amplify the full length GH45 cellulase gene from the plasmid D24H9N for O62FA6 (SEQ ID NO: 5), with two substitutions S221P and S224P corresponding to O42EB3 mature polypeptide. The primers were synthesized by Invitrogen, Beijing, China.

Forward primer D24H9NF:

(SEQ ID NO: 50)

5′ ACACAACTGGGGATC CACC atgcgctctactcccgttcttc 3′
Reverse primer D43NXRlinkerR:

(SEQ ID NO: 77)

5′ ggacggggaggacggg gaggactggcctcccg 3′
Forward primer D43NXRlinkerF:

(SEQ ID NO: 78)

5′ gggaggccagtcctcc ccgtcctccccgtcc 3′
Reverse primer D24H9NR:

(SEQ ID NO: 51)

5′ CCCTCTAGATCTCGAG tcagaggcactgcgagtagtagtc 3′

The capitalized letters are a region homologous to the insertion sites of pCaHj505 (WO2013/029496), and the lower case letters represent coding region of the O141FQ GH45 cellulase. The 4 letters ahead of the coding sequence in forward primer D24H9NF represent the Kozak sequence as the initiation of translation process. The italic letters represent the nucleotides for the mutated amino acid.

Two PCR fragments of 885 bp and 250 bp were individually amplified and purified, and then cloned directly into the expression vector pCaHj505 as described for O62FA8. The DNA sequence and deduced amino acid sequence of the O141FQ mutant polypeptide coding sequence are shown in SEQ ID NO: 35 and SEQ ID NO: 36, respectively. The coding sequence is 1104 bp including the stop codon, which is interrupted by 2 introns of 78 bp (nucleotides 90 to 167) and 69 bp (nucleotides 417 to 485). The encoded protein is 318 amino acids with a signal peptide of 21 residues. The mature protein contains 297 amino acids with a predicted molecular mass of 30 kDa and a predicted isoelectric point of 5.8.

### Example 10

Expression of GH45 Cellulase Variants in Aspergillus oryzae

Aspergillus oryzae strain MT3568 was used for heterologous expression of the GH45 cellulase variant genes. A. oryzae MT3568 is an amdS (acetamidase) disrupted gene derivative of A. oryzae JaL355 (WO02/40694) in which pyrG auxotrophy was restored by disrupting the A. oryzae acetamidase (amdS) gene with the pyrG gene.

Protoplasts were prepared according to the method of Christensen et al., 1988, Bio/Technology 6: 1419-1422. Three μg of each plasmid described in Examples 5-9 was used to transform Aspergillus oryzae MT3568.

The transformation of Aspergillus oryzae MT3568 with each plasmid yielded about 10 transformants. Four transformants were isolated to individual minimal medium plates and were then inoculated separately into 3 ml of YPM medium (1% of Yeast extract, 2% of Peptone and 2% of Maltose) in 24-well plate and incubated at 30° C., 150 rpm. After 3 days incubation, 21 μl of supernatant from each culture were analyzed on NUPAGE® NOVEX® 4-12% Bis-Tris Gel w/MES (Invitrogen Corporation, Carlsbad, Calif., USA) according to the manufacturer's instructions. The resulting gel was stained with INSTANTBLUE™ (Expedeon Ltd., Babraham Cambridge, UK). SDS-PAGE profiles of the cultures showed that the majority of the transformants had a major band of approximately 30 kDa. One of the each kind of transformants was chosen as an expression strain.

### Example 11

### Fermentation of Expression Strains

A slant of the expression strain described in Example 10 was washed with 10 ml of YPM and inoculated into 2-liter flasks each containing 400 ml of YPM medium to generate broth for purification and characterization of the enzyme. The culture was harvested on day 3 and filtered using a 0.22 μm 1-liter bottle top vacuum filter (Corning Inc., Corning, N.Y., USA).

### Example 12

### Purification and Activity Assay of GH45 Cellulase Variants

### O62FA6

The culture supernatant of O62FA6 was firstly precipitated with ammonium sulfate (80% saturation), then dialyzed with 20 mM NaAc at pH4.5. The solution was filtered with 0.45 um filter and then loaded into Q SEPHAROSE® Fast Flow column (GE Healthcare) equilibrated with 20 mM NaAc at pH4.5. A gradient of NaCl concentration was applied as elution buffer from zero to 1 M, and then elution fractions and flow-through fraction were collected to detect cellulase activity. The flow-through fraction with cellulase activity was tried to loaded Mono Q column (GE Healthcare) but the activity still was in the flow-through fraction. The activity was then loaded into SUPERDEX™ 75 column (GE Healthcare) equilibrated with 20 mM Tris-HCl at pH7.0 with 0.3 M NaCl added. Finally, the activity from SUPERDEX™ 75 was added by ammonium sulfate with 1.2 M final concentration in 20 mM Tris-HCl at pH7.0 and then loaded into a chromatographic column with Phenyl Sepharose 6 Fast Flow (GE Healthcare). The activity was eluted by a gradient decrease of salt concentration. The fractions with activity were analyzed by SDS-PAGE and then concentrated for further evaluation.

O62FA8 and O62FAA have similar PI with O62FA6 and the purification procedure is similar with O62FA6, only without Mono Q step.

### O 82DT2

The culture supernatant of O82DT2 was firstly precipitated with ammonium sulfate (80% saturation), then dialyzed with 20 mM Tris-HCl at pH9.0. The solution was filtered with 0.45 um filter and then loaded into Q SEPHAROSE® Fast Flow column (GE Healthcare) equilibrated with 20 mM Tris-HCl at pH9.0. A gradient of NaCl concentration was applied as elution buffer from zero to 1 M, then elution fractions and flowthrough sample were analyzed for cellulase activity. The flow-through sample is the target purified protein.

### O82DT4

The culture supernatant of O82DT4 was added by ammonium sulfate with 1.2 M final concentration and loaded into Phenyl Sepharose 6 Fast Flow column (GE Healthcare) equilibrated with 20 mM PBS pH6.0 with 1.2 M ammonium sulfate added. A gradient decrease of ammonium sulfate concentration from 1.2 M to 0 was set up and the fractions with cellulase activity were analyzed and pooled.

O82AH2, O141FS, O141FK, O141FU, O141FQ, O141FH, O141FF, P446XD, O14G83 were purified by the similar procedure as O82DT4. The target protein could be obtained from flow-through fraction or eluted fractions. The cellulase activity was detected by blue substrate. The blue substrate of AZCL-HE-Cellulose (Megazyme) was dissolved into 100 mM Bis-Tris at pH6.0 with final concentration of 0.2% (w/v). 20 μl enzyme was mixed with 100 μl blue substrate and incubated at 50° C. for 30 min with 600 rpm by Thermomixer (Eppendorf), then 60 μl supernatant of mixture was transferred into a new well to measure absorbance at 595 nm. The variants show good cellulase activities.

### Example 13

Biopolishing with 042EB3, O52EWC in Launder-O-meter

The wild type protein 052EWC (SEQ ID NO: 12) and the fusion protein 042EB3 (SEQ ID NO: 2) purified from Example 4 were used for biopolishing in the present example.

Cotton fabric swatches were cut into about 16 cm * 16 cm (about 5 grams each). The swatches were placed in the conditioned room (65% humidity, 21° C.) for 24 hours before they were numbered, weighed by an analytical balance and recorded. The biopolishing was conducted with a Launder-O-meter. Two conditioned swatches and 20 big steel balls (total weight of 220 grams) or 4 rubber balls were μlaced in each beaker to supply high and low levels of mechanical aids, respectively. The beaker was filled with enzymes according to Table 1 and buffers prepared as described in media part to a total volume of around 100 ml, which could get a liquid to fabric ratio of about 10:1 (v/w).

The Launder-O-Meter (LOM) machine was started after the required program was chosen, and it would hold when the temperature reached the pre-set temperature, 55° C. Each beaker was fitted with a lid lined with 2 neoprin gaskets and close tightly with the metal clamping device. 5 beakers were μlaced in a vertical position, in each of the 4 drum positions. After the treatment at 55° C. for 1 hour with the dosages and mechanical aids as specified in the table below, the swatches were removed from the beakers and transferred into an inactivation solution with 2 g/L of sodium carbonate and kept at 85° C. for 10 min. Then the swatches were rinsed in hot water for 2 times and in cold water for 2 times. And they were tumble-dried for 1 hour, conditioned for 24 hours at 65% relative humidity, 21° C. prior to evaluation in weight loss and pilling notes.

As summarized in Table 1, at high mechanical actions with 20 steel balls in each beaker, the fusion protein O42EB3 delivered marginally better biopolishing performance than O52EWC on the protein basis; while at low mechanical actions with 4 rubber balls in each beaker, O42EB3 was much better than O52EWC by delivering 0.5-0.7 higher pilling notes than O52EWC on the protein basis.

TABLE 1
LOM biobalsting with O42EB3 and O52EWC at 55° C., pH 6.5

| | Dosage | | |
|---|---|---|---|
| Enzyme | (mg/g fabric) | balls in each beaker | Pilling notes |
| O42EB3 | 0.008 | 20 steel balss in each beaker | 2.9 ± 0.2 |
| | 0.016 | | 3.8 ± 0.0 |
| | 0.024 | | 3.8 ± 0.0 |
| O52EWC | 0.008 | | 3.0 ± 0.0 |
| | 0.016 | | 3.4 ± 0.2 |
| | 0.024 | | 3.8 ± 0.0 |
| O42EB3 | 0.008 | 4 rubber balss in each beaker | 2.6 ± 0.2 |
| | 0.016 | | 3.6 ± 0.2 |
| | 0.024 | | 3.4 ± 0.2 |
| O52EWC | 0.008 | | 1.9 ± 0.2 |
| | 0.016 | | 2.9 ± 0.2 |
| | 0.024 | | 2.9 ± 0.2 |

### Example 14

Biopolishing with O82AH2, O82DT2 and O82DT4 in Launder-O-Meter

The wild type protein O82AH2 (SEQ ID NO: 13) and the variant O82DT2 (W292Y, SEQ ID NO: 16) and the fusion protein O82DT4 (SEQ ID NO: 18) purified from Example 12 was used for biopolishing in the present example.

The fabric preparation and trial operation was similar to Example 13. As summarized in Table 2, at high mechanical actions with 20 steel balls in each beaker, O82DT2 and O82DT4 delivered marginally better biopolishing performance than the wild type cellulase O82AH2 on the protein basis; while at low mechanical actions with 4 rubber balls in each beaker, O82DT2 and O82DT4 were much better than O82AH2 by delivering 1.0 and 0.5 higher pilling notes than O82AH2 on the protein basis.

TABLE 2
LOM biobalsting with O82AH2, O82DT2
and O82DT4 at 55° C., pH 6.5

| Enzyme | Dosage (mg/g) | balls in each beaker | Pilling notes |
|---|---|---|---|
| O82DT4 | 0.008 | 20 steel balls | 3.8 ± 0.1 |
| O82AH2 | | | 3.7 ± 0.4 |
| O82DT2 | | | 3.9 ± 0.2 |
| O82DT4 | 0.024 | 4 rubber balls | 3.3 ± 0.1 |
| O82AH2 | | | 2.8 ± 0.3 |
| O82DT2 | | | 3.8 ± 0.1 |

### Example 15

Biopolishing with O42EB3, O62FA6, O62FAA, O62FA8 in Launder-O-Meter

Two groups of variants O42EB3/O62FA6, O62FAA/O62FA8, which has only one amino acid difference within the group, purified from Example 4 and 14 were used for biopolishing in the present example.

The fabric preparation and trial operation was similar to Example 13. As summarized in Table 3, at high mechanical actions with 20 steel balls in each beaker, with the mutation in W292Y in the backbone O42EB3, the resulting enzyme O62FA6 delivered much higher biopolishing performance in term of pilling notes with the same protein dosage, and out of the expectation, to reach the equivalent pilling notes level, the weight loss from O62FA6 was reduced as compared to O42EB3; similarly, the resulting enzyme protein O62FA8 showed significant improvement in better biopolishing performance in pilling notes and reduced weight loss on equivalent pilling notes level.

TABLE 3
LOM bioblasting with O42EB3, O62FA6,
O62FAA, O62FA8 at 55° C., pH 6.5

| Enzyme | Dosage (mg/g) | Weight loss(%) | Pilling notes |
|---|---|---|---|
| O42EB3 | 0.004 | 0.1 ± 0.1 | 1.8 ± 0.0 |
| | 0.008 | 0.1 ± 0.1 | 1.8 ± 0.0 |
| | 0.016 | 0.7 ± 0.2 | 3.5 ± 0.0 |
| O62FA6 | 0.004 | 0.2 ± 0.3 | 2.5 ± 0.0 |
| | 0.008 | 0.7 ± 0.1 | 3.8 ± 0.0 |
| | 0.016 | 1.5 ± 0.0 | 4.0 ± 0.0 |
| O62FAA | 0.004 | −0.1 ± 0.2 | 1.8 ± 0.2 |
| | 0.008 | 0.0 ± 0.2 | 1.6 ± 0.2 |
| | 0.016 | 0.3 ± 0.0 | 2.9 ± 0.2 |
| O62FA8 | 0.004 | −0.1 ± 0.1 | 2.4 ± 0.2 |
| | 0.008 | 0.0 ± 0.2 | 2.5 ± 0.0 |
| | 0.016 | 0.3 ± 0.1 | 3.8 ± 0.0 |

### Example 16

Denim Abrasion with O62FA6 and O52EWC in Wascator

Two proteins O62FA6 purified from Example 12 and O52EWC were used for denim abrasion trials in the present example.

Two types of raw denim were cut and sewn, forming a tube with 35 cm×45 cm in size and 180 grams in weight. Raw denim tubes were desized with amylase according to the product application guideline. The tubes were placed in a conditioned room (65% relative humidity, 21° C.) for 24 hours before they were numbered, weighed by balance and recorded. 5 conditioned tubes (3 tubes of type 1 and 2 tubes of type 2) with small amount of filler to get 1000 g were loaded into wascator (Electrolux Wascator, FOM71CLS). The denim tubes were rinsed with 10 L fresh water at room temperature for 10 min, and then treated with 0.03 mg cellulase protein/g fabric at 55° C. for 60 min in 10 L fresh water and the pH was adjusted to 6-6.5 with acetic acid and/or sodium hydroxide, then rinsed with fresh water for 10 min, twice before they were centrifuged and tumble dried (AEG, LAVATHERM 37700, Germany) for 1 hour, and conditioned.

The abrasion and backstaining level of the denim samples were determined by measuring the reflectance before and after the cellulase treatment with pre-calibrated DataColor SF450X. For both L* and b*, four readings were taken for each fabric and the average of the four readings was used. The abrasion level was evaluated with the index CIE L* of the blue side of the sample, and the backstaining level was evaluated with the index CIE b* of the back of the sample.

As shown in Table 4, at 55° C., pH 6-6.5, with the same protein dosage, O62FA6 led much higher and slightly higher denim abrasion than O52EWC on type 1 and 2 denims, respectively; and on the equivalent abrasion level both cellulase caused similar level of backstaining.

TABLE 4
Denim abrasion by O62FA6 and O52EWC
in wascator at 55° C., 1 hour

| | Dosage | | | |
|---|---|---|---|---|
| Enzyme | (mg/g fabric) | Denim type | L* on front | b* on back |
| O62FA6 | 0.03 | 1 | 23.9 ± 0.2 | −11.3 ± 0.3 |
| | | 2 | 23.4 ± 0.0 | −10.8 ± 0.3 |
| O52EWC | 0.03 | 1 | 23.2 ± 0.0 | −10.9 ± 0.2 |
| | | 2 | 23.3 ± 0.0 | −10.9 ± 0.6 |

### Example 17

Biopolishing with O62FA6 and O52EWC in Wascator

Fermentation broths containing the two proteins mentioned in Example 16 were also tested in wascator for biopolishing of different fabrics. The fermentation broths were measured for CNUR activity and loaded in the present study based on the activity.

In each wascator biopolishing trial, 5 different cotton knits were included: two bleached cotton interlock, and 3 single jersey in blue, brown and navy, respectively. The fabrics were pre-conditioned and weighed as mentioned above. Five types of fabrics summed up to about 1 kg in total in each biopolishing trial in wascator. The fabrics were treated with similar procedure as Example 16 except that in the present example 18 CNUR/g fabric was loaded in the main wash at 55° C. and pH 6-6.5; and one additional inactivation step with 1 g/L sodium carbonate at 80° C. for 10 min was conducted before the two rinse steps. Then the fabrics were centrifuged and tumble dried (AEG, LAVATHERM 37700, Germany) for 1 hour, and conditioned.

As summarized in Table 5, at 55° C., pH 6-6.5, with the same activity dosage, O62FA6 led similar biopolishing performance in pilling notes and a better biopolishing performance with 0.5-1 higher in pilling notes on all the 3 types of dyed fabrics as compared to O52EWC; in weight loss, O62FA6 delivered better performance than O52EWC on ⅗ of the fabrics for less weight loss on equivalent or even higher pilling notes, while on the other 2/5 fabrics, 062FA6 caused a bit higher weight loss in line with higher pilling notes. For strength, for fabrics treated with 062FA6, higher strength was retained on 2/5 fabrics on equivalent or even higher pilling notes, lower strength was retained on 1/5 fabrics on equivalent pilling notes, the last ⅖ with higher pilling notes by 0.5-1 and lower strength loss than O52EWC. Overall to say, with the same activity dosage, O62FA6 delivered better biopolishing performance, less loss in weight and strength.

TABLE 5
Biopolishing by O62FA6 and O52EWC
in wascator at 55° C., 1 hour

| | Dosage | | Pilling | Weight | Strength |
|---|---|---|---|---|---|
| Enzyme | (CNUR/g fabric) | Fabric | notes | loss(%) | (N) |
| O52EWC | 18 | Blue | 3.7 ± 0.2 | 2.0 | 650 ± 35 |
| | | Brown | 1.3 ± 0.3 | −0.9 | 573 ± 37 |
| | | White | 3.9 ± 0.3 | 2.0 | 885 ± 44 |
| | | White | 4.2 ± 0.3 | 2.4 | 912 ± 67 |
| | | normal | | | |
| | | Navy | 1.6 ± 0.5 | −0.4 | 663 ± 28 |
| O62FA6 | 18 | Blue | 4.2 ± 0.3 | 2.3 | 606 ± 40 |
| | | Brown | 2.3 ± 0.4 | −0.4 | 609 ± 17 |
| | | White | 3.9 ± 0.3 | 1.9 | 807 ± 55 |
| | | White | 4.1 ± 0.2 | 1.9 | 955 ± 53 |
| | | normal | | | |
| | | Navy | 2.6 ± 0.6 | −0.3 | 618 ± 49 |

### Example 18

Biopolishing with O62FA6 and O52EWC in Jet Dyer

Fermentation broths mentioned in Example 17 were also tested in jet dyer (Allfit 10, Fong's) for biopolishing of different fabrics.

In each jet dyer biopolishing trial, 2 different cotton knits were included: about 2 kg of cotton interlock and about 1 kg cotton/spandex single jersey. The fabrics were pre-conditioned and weighed as mentioned above. The two types of cotton fabrics and 5 kg cotton fillers summed up to about 8 kg in total in each biopolishing trial in jet dyer. They were rinsed with 80 L fresh water at room temperature for 10 min, and in main wash treated with O62FA6 or O52EWC in 30 CNUR/g fabric at 55oC for 90 min in 80 L fresh water and the pH was adjusted to 6-6.5 with acetic acid and/or sodium hydroxide, fabrics swatches were collected after cellulase was loaded for 75 min and 90 min, respectively; after the main wash with cellulase, drained and inactivated with 1 g/L sodium carbonate in 80 L fresh water at 80° C. for 10 min, rinsed with fresh water for 10 min, twice. In the whole process in jet dyer, the winch speed was set at 40 m/min and suitable pump pressure was set to make sure fabrics ran smoothly in the machine. The treated fabrics were then centrifuged and tumble dried for 1 hour, and conditioned.

As summarized in Table 6, at 55° C., pH 6-6.5, with the same activity dosage, O62FA6 led significantly better biopolishing performance than O52EWC: in 90 min, O62FA6 led higher pilling notes by 0.8 and 0.6 on the two fabrics than O52EWC, respectively; or to reach the pilling notes level of O52EWC in 90 min, it took only 75 min for O62FA6. Besides higher pilling notes level, O62FA6 also retained higher strength than O52EWC.

TABLE 6
Biopolishing by O62FA6 and O52EWC
in jet dyer at 55° C., 90 min

| | Dosage | | 75 min | 90 min | |
|---|---|---|---|---|---|
| | (CNUR/ | | Pilling | Pilling | Strength |
| Enzyme | g fabric) | Fabric | notes | notes | (N) |
| O62FA6 | 30 | Cotton | 2.6 ± 0.1 | 3.6 ± 0.1 | 979 ± 51 |
| | | interlock | | | |
| | | Cotton/ | 1.8 ± 0.3 | 3.5 ± 0.1 | 433 ± 19 |
| | | spandex | | | |
| | | single jersey | | | |
| O52EWC | 30 | Cotton | 1.8 ± 0.3 | 2.8 ± 0.4 | 930 ± 65 |
| | | interlock | | | |
| | | Cotton/ | 1.5 ± 0.4 | 1.9 ± 0.4 | 429 ± 22 |
| | | spandex | | | |
| | | single jersey | | | |

### Example 19

Biopolishing with O14G83, O52EWC in Launder-O-Meter

The purified protein O14G83 from Example 12 and fermentation broth O52EWC mentioned in Example 17 were used for biopolishing trials in the present example.

The fabric preparation and trial operation was similar to Example 13. As summarized in Table 7, at high mechanical actions with 20 steel balls in each beaker, the fusion protein O14G83 delivered better biopolishing performance with higher pilling notes while similar weight loss than O52EWC when same protein was loaded.

TABLE 7
LOM biobalsting with O14G83 and O52EWC at 55° C., pH 6.5

| Enzyme | Dosage (mg/g fabric) | Weight loss (%) | Pilling notes |
|---|---|---|---|
| O14G83 | 0.004 | 0.6 ± 0.0 | 3.0 ± 0.0 |
| | 0.016 | 1.4 ± 0.1 | 4.0 ± 0.3 |
| O52EWC | 0.004 | 0.5 ± 0.1 | 2.7 ± 0.3 |
| | 0.016 | 1.4 ± 0.1 | 3.5 ± 0.0 |

### Example 20

Biopolishing with O141FN, O141FS, O141FK, O141FU and O52EWC in Launder-O-Meter

The purified protein O141FN, O141FS, O141FK, P141FU from Example 12 and fermentation broth O52EWC mentioned in Example 16 were used for biopolishing trials in the present example.

The fabric preparation and trial operation was similar to Example 13. As summarized in Table 8, at high mechanical actions with 20 steel balls in each beaker, all the 4 fusion proteins tested here delivered better biopolishing performance with higher pilling notes than O52EWC when same protein was loaded.

TABLE 8
LOM biobalsting with O141FN, O141FS, O141FK,
O141FU and O52EWC at 55° C., pH 6.5

| Enzyme | Dosage (mg/g fabric) | Weight loss (%) | Pilling notes |
|---|---|---|---|
| O141FN | 0.002 | 1.1 ± 0.0 | 3.1 ± 0.2 |
| | 0.008 | 2.1 ± 0.1 | 3.9 ± 0.2 |
| O141FS | 0.002 | 0.5 ± 0.2 | 3.3 ± 0.0 |
| | 0.008 | 1.7 ± 0.1 | 3.9 ± 0.2 |
| O141FK | 0.002 | 0.7 ± 0.2 | 2.8 ± 0.0 |
| | 0.008 | 1.9 ± 0.0 | 3.9 ± 0.2 |
| O141FU | 0.002 | 1.0 ± 0.2 | 2.6 ± 0.2 |
| | 0.008 | 2.0 ± 0.1 | 4.1 ± 0.2 |
| O52EWC | 0.002 | 0.5 ± 0.1 | 2.6 ± 0.2 |
| | 0.008 | 1.2 ± 0.2 | 3.6 ± 0.2 |

### Example 21

Biopolishing with O141FF, O141FH, O141FQ and O52EWC in Launder-O-Meter

The purified protein O141FF, O141FH, O141FQ from Example 12 and fermentation broth O52EWC mentioned in Example 16 were used for biopolishing trials in the present example.

The fabric preparation and trial operation was similar to Example 13. As summarized in Table 9, at high mechanical actions with 20 steel balls in each beaker, all the 3 fusion proteins tested here delivered better biopolishing performance with higher pilling notes than O52EWC when same protein was loaded; at low mechanical actions with 4 rubber balls in each beaker, O141FH delivered significantly higher pilling notes than O52EWC and the other fusion proteins.

TABLE 9
LOM biobalsting with O141FF, O141FH,
O141FQ and O52EWC at 55° C., pH 6.5

| | Dosage | | Weight loss | Pilling |
|---|---|---|---|---|
| Enzyme | (mg/g) | balls in each beaker | (%) | notes |
| O141FQ | 0.002 | 20 steel balls | 0.5 ± 0.1 | 3.4 ± 0.2 |
| O141FH | | | 0.1 ± 0.2 | 3.6 ± 0.2 |
| O141FF | | | 0.2 ± 0.1 | 3.6 ± 0.2 |
| O52EWC | | | 0.0 ± 0.1 | 2.4 ± 0.2 |
| O141FQ | 0.024 | 4 rubber balls | 1.5 ± 0.1 | 3.1 ± 0.5 |
| O141FH | | | 1.5 ± 0.1 | 3.7 ± 0.1 |
| O141FF | | | 1.4 ± 0.1 | 3.0 ± 0.2 |
| O52EWC | | | 1.1 ± 0.1 | 3.1 ± 0.3 |

### Example 22

Biopolishing with P24YEZ, P446XD in Launder-O-Meter

Two proteins P24YEZ (SEQ ID NO: 14) and P446XD purified from Example 12 were used for biopolishing trials in the present example.

The fabric preparation and trial operation was similar to Example 13. As summarized in Table 10, at high mechanical actions with 20 steel balls in each beaker, with the mutation in Y292W in the backbone P24YEZ, the resulting enzyme P446XD delivered similar biopolishing performance in term of pilling notes with the same protein dosage, and out of the expectation, to reach the equivalent pilling notes level, the weight loss from P446XD was greatly increased as compared to P24YEZ. For example, to reach the pilling notes level 3.8 at pH 7.5, the weight loss was 1.5% and 0.8% by P446XD and P24YEZ, respectively.

TABLE 10
LOM biobalsting with P24YEZ, P446XD at 35° C., pH 7.5

| | | Dosage | Weight loss | Pilling |
|---|---|---|---|---|
| Enzyme | PH | (mg/g fabric) | (%) | notes |
| P24YEZ | 6.5 | 0.008 | 0.2 ± 0.0 | 3.4 ± 0.2 |
| | | 0.032 | 1.2 ± 0.1 | 3.6 ± 0.2 |
| | 7.5 | 0.008 | 0.1 ± 0.1 | 3.3 ± 0.0 |
| | | 0.032 | 0.8 ± 0.1 | 3.8 ± 0.0 |
| P446XD | 6.5 | 0.008 | 0.4 ± 0.2 | 3.5 ± 0.0 |
| | | 0.032 | 1.4 ± 0.3 | 3.8 ± 0.0 |
| | 7.5 | 0.008 | 0.5 ± 0.0 | 3.1 ± 0.2 |
| | | 0.032 | 1.5 ± 0.2 | 3.8 ± 0.0 |

### Example 23

Biopolishing with O32JRC, O52EWC in Launder-O-Meter

Two proteins O32JRC purified from Example 4 and O52EWC and were used for biopolishing trials in the present example.

The fabric preparation and trial operation was similar to Example 13 except that in current example both high mechanical action with 20 steel balls and low mechanical action with 2 rubber balls were tested and samples were collected after cellulase treatment for 30, 60 and 90 minutes, respectively. As summarized in Table 11, with both high and low mechanical actions, O32JRC delivered better biopolishing performance than O52EWC for higher pilling notes than O52EWC during the whole process from 30 to 90 min.

TABLE 11
LOM biobalsting with O32JRC, O52EWC at 55° C., pH 6.5

| | Dosage | | Time | |
|---|---|---|---|---|
| Enzyme | (mg/g) | Balls in each beaker | (min) | Pilling notes |
| Blank | 0 | 2 rubber balls | 90 | 1.5 ± 0.0 |
| O32JRC | 0.016 | 20 steel balls | 30 | 1.9 ± 0.2 |
| | | | 60 | 3.8 ± 0.0 |
| | | | 90 | 3.9 ± 0.2 |
| | 0.064 | 2 rubber balls | 30 | 1.9 ± 0.2 |
| | | | 60 | 3.4 ± 0.2 |
| | | | 90 | 3.6 ± 0.2 |
| O52EWC | 0.016 | 20 steel balls | 30 | 1.6 ± 0.2 |
| | | | 60 | 3.4 ± 0.2 |
| | | | 90 | 3.3 ± 0.0 |
| | 0.064 | 2 rubber balls | 30 | 1.6 ± 0.2 |
| | | | 60 | 3.1 ± 0.2 |
| | | | 90 | 2.9 ± 0.2 |

The invention described and claimed herein is not to be limited in scope by the specific aspects herein disclosed, since these aspects are intended as illustrations of several aspects of the invention. Any equivalent aspects are intended to be within the scope of this invention.

Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1-110. (canceled)

111. A variant of a parent cellulase, wherein the variant comprises an alteration at one or more positions corresponding to positions 292, 274, 266, 265, 255, 246, 237, 224 and 221 of the mature polypeptide of SEQ ID NO: 2, and wherein the variant has cellulase activity.

112. The variant of claim 111, wherein the cellulase is GH45 cellulase.

113. The variant of claim 111, wherein the alteration is an insertion or deletion or substitution.

114. The variant of claim 111, wherein the variant comprises a catalytic domain and a cellulase binding domain, and wherein the cellulase binding domain is heterologous to the catalytic domain.

115. The variant of claim 111, which is a variant of a parent cellulase selected from the group consisting of:
a. a polypeptide having at least 60% sequence identity to the mature polypeptide of SEQ ID NO: 2, SEQ ID NO: 4, SEQ ID NO: 8, SEQ ID NO: 12, SEQ ID NO: 13, SEQ ID NO: 14; or SEQ ID NO: 18;
b. a polypeptide encoded by a polynucleotide that hybridizes under low stringency conditions with (i) the mature polypeptide coding sequence of SEQ ID NO: 1, SEQ ID NO: 3, SEQ ID NO: 7, SEQ ID NO: 37, SEQ ID NO: 38, SEQ ID NO: 39 or SEQ ID NO: 17, (ii) the cDNA sequence thereof, or (iii) the full-length complement of (i) or (ii);
c. a polypeptide encoded by a polynucleotide having at least 60% identity to the mature polypeptide coding sequence of SEQ ID NO: 1, SEQ ID NO: 3, SEQ ID NO: 7, SEQ ID NO: 37, SEQ ID NO: 38, SEQ ID NO: 39 or SEQ ID NO: 17, or the cDNA sequence thereof; and
d. a fragment of the mature polypeptide of SEQ ID NO: 2, SEQ ID NO: 4, SEQ ID NO: 8, SEQ ID NO: 12, SEQ ID NO: 13, SEQ ID NO: 14; or SEQ ID NO: 18, which has cellulase activity.

116. The variant of claim 111, wherein the variant has at least 60%, e.g., at least 65%, at least 70%, at least 75%, at least 80%, at least 85%, at least 90%, at least 95% identity, at least 96%, at least 97%, at least 98%, or at least 99%, but less than 100%, sequence identity to the amino acid sequence of the parent cellulase.

117. The variant of claim 111, wherein the variant comprises one or more substitutions selected from the group consisting of W292Y, F274Y, W266Y, R265K, S255P, T246N, S224P, S224P and S221P.

118. The variant of claim 117, wherein the variant comprises one or more substitutions selected from the group consisting of
W292Y
W266Y+W292Y
R265K+W292Y
R265K+W266Y+W292Y
F274Y+W292Y
W266Y+F274Y+W292Y
S221 P+S224P+W292Y
S224P+T246N+W292Y
S255P+W292Y and
W266Y+W292Y.

119. The variant of claim 111, wherein the variant has an improved property relative to the parent, and wherein the improved property is improved biofinishing performance, a reduced weight loss of cellulose-containing textile, and/or a reduced loss in cellulose- containing textile strength.

120. A variant of a parent GH45 cellulase, wherein the variant comprises a catalytic domain and a cellulose binding domain, wherein the cellulase binding domain is heterologous to the catalytic domain, and wherein the variant has an improved biofinishing activity compared with the parent GH45 cellulase.

121. The variant of claim 120, wherein the catalytic domain is selected from the group consisting of:
a. a polypeptide having at least 60% sequence identity to the catalytic domain of SEQ ID NO: 12, SEQ ID NO: 13, or SEQ ID NO: 14;
b. a polypeptide encoded by a polynucleotide that hybridizes under low stringency conditions with (i) the catalytic domain coding sequence of SEQ ID NO: 37, SEQ ID NO: 38, or SEQ ID NO: 39, (ii) the cDNA sequence thereof, or (iii) the full-length complement of (i) or (ii);
c. a polypeptide encoded by a polynucleotide having at least 60% identity to the catalytic domain coding sequence of SEQ ID NO: 37, SEQ ID NO: 38, SEQ ID NO: 39, or the cDNA sequence thereof; and
d. a fragment of the catalytic domain of SEQ ID NO: 12, SEQ ID NO: 13, or SEQ ID NO: 14, which has cellulase activity.

122. The variant of claim 120, wherein the cellulose binding domain is selected from the group consisting of:
a. a polypeptide having at least 60% sequence identity to the cellulose binding domain of SEQ ID NO: 12, SEQ ID NO: 13, SEQ ID NO: 14 or SEQ ID NO: 8;
b. a polypeptide encoded by a polynucleotide that hybridizes under low stringency conditions with (i) the cellulose binding domain coding sequence of SEQ ID NO: 37, SEQ ID NO: 38, SEQ ID NO: 39 or SEQ ID NO: 7, (ii) the cDNA sequence thereof, or (iii) the full-length complement of (i) or (ii);
c. a polypeptide encoded by a polynucleotide having at least 60% identity to the cellulose binding domain coding sequence of SEQ ID NO: 37, SEQ ID NO: 38, SEQ ID NO: 39 or SEQ ID NO: 7, or the cDNA sequence thereof; and
d. a fragment of the cellulose binding domain of SEQ ID NO: 12, SEQ ID NO: 13, SEQ ID NO: 14 or SEQ ID NO: 8, which has the cellulose binding activity.

123. The variant of claim 120, wherein the variant comprises a linker between the catalytic domain and cellulose binding domain, and wherein the linker is selected from the group consisting of:
a. a polypeptide having at least 60% sequence identity to the linker of SEQ ID NO: 12, SEQ ID NO: 13, SEQ ID NO: 14 or SEQ ID NO: 8;
b. a polypeptide encoded by a polynucleotide that hybridizes under low stringency conditions with (i) the linker coding sequence of SEQ ID NO: 37, SEQ ID NO: 38, SEQ ID NO: 39 or SEQ ID NO: 7, (ii) the cDNA sequence thereof, or (iii) the full-length complement of (i) or (ii);
c. a polypeptide encoded by a polynucleotide having at least 60% identity to the linker coding sequence of SEQ ID NO: 37, SEQ ID NO: 38, SEQ ID NO: 39 or SEQ ID NO: 7, or the cDNA sequence thereof; and
d. a fragment of the linker of SEQ ID NO: 12, SEQ ID NO: 13, SEQ ID NO: 14 or SEQ ID NO: 8, which has the cellulose binding activity.

124. A polynucleotide encoding the variant of claim 111 or claim 120.

125. A nucleic acid construct, an expression vector, or a host cell comprising the polynucleotide of claim 14.

126. A process for biofinishing, comprising treating a cellulose-containing textile with the variant of claim 111 or claim 120.
